(19) United States
(12) Patent Application Publication
(10) Pub. No.: US 2010/0273186 A1
(43) Pub. Date: Oct. 28, 2010

(54) SPLIT MUTANT HYDROLASE FUSION REPORTER AND USES THEREOF

(75) Inventor: Keith V. Wood, Mt. Horeb, WI (US)

Correspondence Address: Michael Best & Friedrich LLP, 100 East Wisconsin Avenue, Suite 3300, Milwaukee, WI, 53202, US

(73) Assignee: Promega Corporation, Madison, WI (US)

(21) Appl. No.: 12/501,249

(22) Filed: Jul. 10, 2009

Related U.S. Application Data
(63) Continuation of application No. PCT/US2008/000376, filed on Jan. 10, 2008.
(60) Provisional application No. 60/985,583, filed on Nov. 5, 2007.
(60) Provisional application No. 60/879,701, filed on Jan. 10, 2007.

(51) Int. Cl. G01N 33/573; C07H 21/04; C12N 15/63

(52) U.S. Cl. 435/7.4; 536/23.2; 435/320.1

(57) ABSTRACT

The invention provides polynucleotides encoding and polypeptides corresponding to split hydrolase fusion proteins, wherein the hydrolase sequence may include at least one substitution, and use of the split hydrolase fusion proteins.

### CROSS-REFERENCE TO RELATED APPLICATIONS

The present application claims the benefit of the filing date of U.S. application Ser. No. 60/985,583, filed Nov. 5, 2007 and U.S. application Ser. No. 60/879,701, filed Jan. 10, 2007, the disclosures of which are incorporated by reference herein.

### BACKGROUND

Luciferase biosensors have been described. For example, Sala-Newby et al. (1991) disclose that a Photinus pyralis luciferase cDNA was amplified in vitro to generate cyclic AMP-dependent protein kinase phosphorylation sites. In particular, a valine at position 217 was mutated to arginine to generate a site, RRFS (SEQ ID NO:11), and the heptapeptide kemptide, the phosphorylation site of the porcine pyruvate kinase, was added at the N- or C-terminus of the luciferase. Sala-Newby et al. relate that the proteins carrying phosphorylation sites were characterized for their specific activity, pI, effect of pH on the color of the light emitted, and effect of the catalytic subunit of protein kinase A in the presence of ATP. They found that only one of the recombinant proteins (RRFS; SEQ ID NO:11) was significantly different from wild type luciferase and that the RRFS (SEQ ID NO:11) mutant had a lower specific activity, lower pH optimum, emitted greener light at low pH and, when phosphorylated, decreased its activity by up to 80%. It is disclosed that the latter effect was reversed by phosphatase.

Waud et al. (1996) engineered protein kinase recognition sequences and proteinase sites into a Photinus pyralis luciferase cDNA. Two domains of the luciferase were modified by Waud et al.; one between amino acids 209 and 227 and the other at the C-terminus, between amino acids 537 and 550. Waud et al. disclose that the mutation of amino acids between residues 209 and 227 reduced bioluminescent activity to less than 1% of wild type recombinant, while engineering peptide sequences at the C-terminus resulted in specific activities ranging from 0.06%-120% of the wild type recombinant luciferase. Waud et al. also disclose that addition of a cyclic AMP dependent protein kinase catalytic subunit to a variant luciferase incorporating the kinase recognition sequence, LRRASLG (SEQ ID NO:12), with a serine at amino acid position 543, resulted in a 30% reduction activity. Alkaline phosphatase treatment restored activity. Waud et al. further disclose that the bioluminescent activity of a variant luciferase containing a thrombin recognition sequence, LVPRES (SEQ ID NO:2), with the cleavage site positioned between amino acids 542 and 543, decreased by 50% when incubated in the presence of thrombin.

Ozawa et al. (2001) describe a biosensor based on protein splicing-induced complementation of rationally designed fragments of firefly luciferase. Protein splicing is a posttranslational protein modification through which inteins (internal proteins) are excised out from a precursor fusion protein, ligating the flanking exteins (external proteins) into a contiguous polypeptide. It is disclosed that the N- and C-terminal intein DnaE from Synechocystis sp. PCC6803 were each fused respectively to N- and C-terminal fragments of a luciferase. Protein-protein interactions trigger the folding of DnaE intein, resulting in protein splicing, and thereby the extein of ligated luciferase recovers its enzymatic activity. Ozawa et al. disclose that the interaction between known binding partners, phosphorylated insulin receptor substrate 1 (IRS-1) and its target N-terminal SH2 domain of PI 3-kinase, was monitored using a split luciferase in the presence insulin.

Paulmurugan et al. (2002) employed a split firefly luciferase-based assay to monitor the interaction of two proteins, i.e., MyoD and Id, in cell cultures and in mice using both complementation strategy and an intein-mediated reconstitution strategy. To retain reporter activity, in the complementation strategy, fusion proteins need protein interaction, i.e., via the interaction of the protein partners MyoD and Id, while in the reconstitution strategy, the new complete beetle luciferase formed via intein-mediated splicing maintains it activity even in the absence of a continuing interaction between the protein partners.

A protein fragment complementation assay is disclosed in Michnick et al. (U.S. Pat. Nos. 6,270,964, 6,294,330 and 6,428,951). Specifically, Michnick describe a split murine dihydrofolate reductase (DHFR) gene-based assay in which an N-terminal fragment of DHFR and a C-terminal fragment of DHFR are each fused to a GCN4 leucine zipper sequence. DHFR activity was detected in cells which expressed both fusion proteins. Michnick et al. also describe another complementation approach in which nested sets of S1 nuclease generated deletions in the aminoglycoside kinase (AK) gene are introduced into a leucine zipper construct, and the resulting sets of constructs introduced to cells and screened for AK activity.

Moreover, certain enzymes can be circularly permuted and may retain activity (see, e.g., Cheltsov et al., 2003, Jougard et al., 2002, and Nagai et al., 2001).

Thus, enzymes may retain catalytic activity even when their structures are substantially altered by, for example, circularly permuting their amino acid sequence or splitting the enzyme into two fragments.

### SUMMARY OF THE INVENTION

Split mutant proteins, i.e., enzymes mutated to inhibit or eliminate catalytic activity, may be useful in revealing and analyzing protein interaction within cells, e.g., where each portion (fragment) of the split protein is fused to a different protein. The invention provides for split mutated hydrolases, such as those derived from mutated hydrolases disclosed in U.S. published application 20060024808, the disclosure of which is incorporated by reference herein. Even though these mutant hydrolases are not enzymes, the stable binding of a substrate thereto is dependent on proper protein structure. The consequence of re-associating the split fragments of a mutated hydrolase differs from that of a split enzyme system because the labeling function of a mutated hydrolase is retained on one of the fragments even after it has separated from its partner, whereas split enzymes are only active while they are brought together. In effect, the labeling reaction of a split mutant hydrolase provides a molecular memory of a protein interaction.

As an example of a mutated hydrolase, a mutated dehalogenase provides for efficient labeling within a living cell or lysate thereof. This labeling is only conditional on expression of the protein and the presence of the labeled hydrolase substrate. In contrast, the labeling of a split mutant dehalogenase is dependent on a specific protein interaction occurring within the cell and the presence of the labeled hydrolase substrate. For instance, beta-arrestin may be fused with one fragment of a mutated hydrolase, and a G-coupled receptor may be fused with the other fragment. Upon receptor stimulation in the presence of the labeled substrate, beta-arrestin binds to the receptor causing a labeling reaction of either the receptor or the beta-arrestin (depending on which portion of the mutated hydrolase contains the reactive nucleophilic amino acid). A “fragment” of a hydrolase as used herein is a sequence which is less than the full length sequence but which alone cannot form a substrate binding site, and/or has substantially reduced or no substrate binding activity but which, in close proximity to a second fragment of a hydrolase, exhibits substantially increased substrate binding activity. In one embodiment, a fragment of a hydrolase is at least 20, e.g., at least 50, contiguous residues of a wild type hydrolase or a mutated hydrolase, and may not necessarily include the N-terminal or C-terminal residue or N-terminal or C-terminal sequences of the corresponding full length protein.

The invention thus provides a split mutant hydrolase system which includes a first fragment of a hydrolase fused to a protein of interest and a second fragment of the hydrolase optionally fused to a ligand of the first protein of interest. At least one of the hydrolase fragments has a substitution that if present in a full length mutant hydrolase having the sequence of the two fragments, forms a bond with a hydrolase substrate which is more stable than the bond formed between the corresponding full length wild type hydrolase and the hydrolase substrate. In one embodiment, each fragment of the hydrolase is fused to a protein of interest and the proteins of interest interact, e.g., bind to each other. In another embodiment, one hydrolase fragment is fused to a protein of interest which interacts with a molecule in a sample. In another embodiment, in the presence of an agent (one or more agents of interest), or under certain conditions, a complex is formed by the binding of a fusion having the protein of interest fused to a first hydrolase fragment, to a second protein fused to a second hydrolase fragment or to the second hydrolase fragment and a cellular molecule.

Thus, the two fragments of the hydrolase together provide a mutant hydrolase that is structurally related to (substantially corresponds in sequence to) a full length wild type (native) hydrolase but includes at least one amino acid substitution, and in some embodiments at least two amino acid substitutions, relative to the corresponding full length wild type hydrolase. The full length mutant hydrolase lacks or has reduced catalytic activity relative to the corresponding full length wild type hydrolase, and specifically binds substrates which may be specifically bound by the corresponding full length wild type hydrolase, however, no product or substantially less product, e.g., 2-, 10-, 100-, or 1000-fold less, is formed from the interaction between the mutant hydrolase and the substrate under conditions which result in product formation by a reaction between the corresponding full length wild type hydrolase and substrate. The lack of, or reduced amounts of, product formation by the mutant hydrolase is due to at least one substitution in the full length mutant hydrolase, which substitution results in the mutant hydrolase forming a bond with the substrate which is more stable than the bond formed between the corresponding full length wild type hydrolase and the substrate.

Preferably, the bond formed between a substrate and the full length mutant hydrolase or the two associated fragments thereof, and the bond to one of the fragments after disassociation of the two fragments, has a half-life (i.e., t_{1/2}) that is greater than, e.g., at least 2-fold, and more preferably at least 4- or even 10-fold, and up to 100-, 1000- or 10,000-fold greater or more, than the t_{1/2} of the bond formed between a corresponding full length wild type hydrolase and the substrate under conditions which result in product formation by the corresponding full length wild type hydrolase. Preferably, the bond formed between a substrate and the full length mutant hydrolase or associated two fragments thereof, and the bond to one of the fragments after disassociation of the two fragments, has a t_{1/2} of at least 30 minutes and preferably at least 4 hours, and up to at least 10 hours, and is resistant to disruption by washing, protein denaturants, and/or high temperatures, e.g., the bond is stable to boiling in SDS.

The amino acid sequence of at least one end of a hydrolase fragment of the invention is at a site (residue) or in a region which is tolerant to modification, e.g., tolerant to an insertion, a deletion, circular permutation, or any combination thereof. Thus, in one embodiment, the invention includes a system having two fragments of a hydrolase with a N- or C-terminus at a residue corresponding to a residue in a region including residue 14 to 24, residue 25 to 35, residue, 52 to 62, residue 73 to 83, residue 93 to 103, residue 131 to 141, residue 149 to 159, residue 175 to 185, residue 190 to 200, residue 204 to 220, residue 230 to 268, or residue 289 to 299 of a dehalogenase. Corresponding positions may be identified by aligning hydrolase sequences. In one embodiment the invention includes a system having two fragments of a hydrolase with a N- or C-terminus at a residue in a region corresponding to residue 73 to 83, 93 to 103, or 204 to 220 of a dehalogenase such as DhaA. For instance, one end of the hydrolase fragment corresponds to a site or region internal to the N- or C-terminus of the full length mutant or full length wild type hydrolase and the other may be at or near the N- or C-terminus of the full length hydrolase sequence. For instance, each fragment of the hydrolase may include deletions at its N- or C-terminus of 1 to about 10 or 15 residues, or any integer in between, relative to the sequence of a corresponding full length mutant or wild type hydrolase. The N- and/or C-terminus of the hydrolase fragment may be modified by the addition of residues, e.g., an insertion of one or more amino acid residues and optionally hydrolase sequences also found in a second hydrolase fragment to be employed in the compositions and methods of the invention, thereby yielding a fusion protein. The additional sequences may include a heterologous amino acid sequence which is selected to directly or indirectly interact with a molecule of interest (e.g., a cellular protein). In one embodiment, a hydrolase fragment is fused to 4 or more, e.g., 5, 10, 20, 50, 100, 200, 300 or more, but less than about 1000, e.g., about 700, or any integer in between, heterologous amino acid residues. In one embodiment, a hydrolase fragment includes 5%, 10%, 15%, 25%, 33% or 50% or more of the full length hydrolase sequence, e.g., 1 to 20 residues, 1 to 50 residues, 1 to 75 residues, 1 to 100 residues, 1 to 125 residues, or 1 to any integer from 50 to 125, of the full length hydrolase sequence. In one embodiment, one fragment of a hydrolase which is a dehalogenase corresponds to the N-terminal 20, 50, 75, 100, 150, 200, or 250, or any integer in between, residues of a full length wild type or mutant dehalogenase, while the other fragment substantially corresponds to the remaining C-terminal sequence. For instance, in one embodiment, one fragment of the dehalogenase corresponds to the C-terminal 50, 75, 100, 150, 200, or 250, or any integer in between, residues of a full length dehalogenase, which the other fragment substantially corresponds to the remaining N-terminal sequence of the dehalogenase.

In one embodiment, both fragments of the hydrolase are fused to heterologous sequences. In one embodiment, the heterologous sequences are substantially the same and specifically bind to each other, e.g., form a dimer, optionally in the absence of one or more exogenous agents. In another embodiment, the heterologous sequences are different and specifically bind to each other, optionally in the absence of one or more exogenous agents. In one embodiment, one hydrolase fragment is fused to a heterologous sequence and that heterologous sequence interacts with a cellular molecule. In another embodiment, each hydrolase fragment is fused to a heterologous sequence and in the presence of one or more exogenous agents or under specified conditions, the heterologous sequences interact. For instance, in the presence of rapamycin, a fragment of a hydrolase fused to rapamycin binding protein (FRB) and another fragment fused to FK506 binding protein (FKBP), yields a complex of the two fusion proteins. In one embodiment, in the presence of the exogenous agent(s) or under different conditions, the complex of fusion proteins does not form. In one embodiment, one heterologous sequence includes a domain, e.g., 3 or more amino acid residues, which optionally may be covalently modified, e.g., phosphorylated, that noncovalently interacts with a domain in the other heterologous sequence. The two fragments of the hydrolase, at least one of which is fused to a protein of interest, may be employed to detect reversible interactions, e.g., binding of two or more molecules, or other conformational changes or changes in conditions, such as pH, temperature or solvent hydrophobicity, or irreversible interactions.

Heterologous sequences useful in the invention include but are not limited to those which interact in vitro and/or in vivo. For instance, the fusion protein may comprise a fragment of hydrolase and an enzyme of interest, e.g., luciferase, RNasin or RNase, and/or a channel protein, a receptor, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, a transcription factor, a transporter protein and/or a targeting sequence, e.g., a myristilation sequence, a mitochondrial localization sequence, or a nuclear localization sequence, that directs the hydrolase fragment, for example, a fusion protein, to a particular location. The protein of interest, which is fused to a hydrolase fragment, may be a fragment of a wild-type protein, e.g., a functional or structural domain of a protein, such as a domain of a kinase, a transcription factor, and the like. The protein of interest may be fused to the N-terminus or the C-terminus of the hydrolase fragment. In one embodiment, the fusion protein comprises a protein of interest at the N-terminus, and another protein, e.g., a different protein, at the C-terminus, of the hydrolase fragment. For example, the protein of interest may be an antibody. Optionally, the proteins in the fusion are separated by a connector sequence, e.g., preferably one having at least 2 amino acid residues, such as one having 13 to 17 amino acid residues. The presence of a connector sequence in a fusion protein of the invention does not substantially alter the function of either protein in the fusion relative to the function of each individual protein. For any particular combination of proteins in a fusion, a wide variety of connector sequences may be employed. In one embodiment, the connector sequence is a sequence recognized by an enzyme, e.g., a cleavable sequence, or is a photocleavable sequence.

Exemplary heterologous sequences include but are not limited to sequences such as those in FRB and FKBP, the regulatory subunit of protein kinase (PKa-R) and the catalytic subunit of protein kinase (PKa-C), a src homology region (SH2) and a sequence capable of being phosphorylated, e.g., a tyrosine containing sequence, an isoform of 14-3-3, e.g., 14-3-3t (see Mils et al., 2000), and a sequence capable of being phosphorylated, a protein having a WW region (a sequence in a protein which binds proline rich molecules (see Ilsley et al., 2002; and Einbond et al., 1996) and a heterologous sequence capable of being phosphorylated, e.g., a serine and/or a threonine containing sequence, as well as sequences in dihydrofolate reductase (DHFR) and gyrase B (GyrB).

The invention also provides an isolated nucleic acid molecule (polynucleotide) comprising a nucleic acid sequence encoding a fragment of a hydrolase. Further provided is an isolated nucleic acid molecule comprising a nucleic acid sequence encoding a fusion protein comprising a fragment of a hydrolase and one or more amino acid residues at the N-terminus (a N-terminal fusion partner) and/or C-terminus (a C-terminal fusion partner) of the fragment. In one embodiment, the fusion protein comprises at least two different fusion partners, one at the N-terminus and another at the C-terminus, where one of the fusions may be a sequence used for purification, e.g., a glutathione S-transferase (GST) or a polyHis sequence, a sequence intended to alter a property of the remainder of the fusion protein, e.g., a protein destabilization sequence, or a sequence which has a property which is distinguishable. In one embodiment, the isolated nucleic acid molecule comprises a nucleic acid sequence which is optimized for expression in at least one selected host. Optimized sequences include sequences which are codon optimized, i.e., codons which are employed more frequently in one organism relative to another organism, e.g., a distantly related organism, as well as modifications to add or modify Kozak sequences and/or introns, and/or to remove undesirable sequences, for instance, potential transcription factor binding sites. In one embodiment, the polynucleotide includes a nucleic acid sequence encoding a fragment of dehalogenase, which nucleic acid sequence is optimized for expression in a selected host cell. In one embodiment, the optimized polynucleotide no longer hybridizes to the corresponding non-optimized sequence, e.g., does not hybridize to the non-optimized sequence under medium or high stringency conditions. In another embodiment, the polynucleotide has less than 90%, e.g., less than 80%, nucleic acid sequence identity to the corresponding non-optimized sequence and optionally encodes a polypeptide having at least 80%, e.g., at least 85%, 90% or more, amino acid sequence identity with the polypeptide encoded by the non-optimized sequence.

Constructs, e.g., expression cassettes, and vectors comprising the isolated nucleic acid molecule, as well as host cells having one or more of the constructs, and kits comprising the isolated nucleic acid molecule, one or more constructs or vectors are also provided. Host cells include prokaryotic cells or eukaryotic cells such as a plant or vertebrate cells, e.g., mammalian cells, including but not limited to a human, non-human primate, canine, feline, bovine, equine, ovine or rodent (e.g., rabbit, rat, ferret or mouse) cell. Preferably, the expression cassette comprises a promoter, e.g., a constitutive or regulatable promoter, operably linked to the nucleic acid molecule. In one embodiment, the expression cassette contains an inducible promoter. In one embodiment, the invention includes a vector comprising a nucleic acid sequence encoding a fusion protein comprising a fragment of a dehalogenase. Optionally, optimized nucleic acid sequences, e.g., human codon optimized sequences, encoding at least a fragment of the hydrolase, and preferably the fusion protein comprising the fragment of a hydrolase, are employed in the nucleic acid molecules of the invention. The optimization of nucleic acid sequences is known to the art, see, for example WO 02/16944.

In one embodiment, the invention provides a composition having a first polynucleotide, e.g., an expression vector, comprising an open reading frame for a first fusion protein having a first fragment of a hydrolase, e.g., a dehalogenase, and a first heterologous amino acid sequence. The first fragment of the hydrolase includes at least 20 contiguous amino acid residues of a full length hydrolase which residues are capable of associating with a second fragment of a hydrolase, wherein the complex formed by the association of the two fragments, but not the first hydrolase fragment or the second hydrolase fragment, is capable of stably binding a hydrolase substrate for a corresponding full length, wild type hydrolase. The N- and/or C-termini of the first and second hydrolase fragments are at a residue or in a region in a full length wild type hydrolase sequence which is tolerant to modification, and wherein the first heterologous amino acid sequence is selected to directly or indirectly interact with a molecule of interest. In one embodiment, the hydrolase is a mutant dehalogenase having a substitution at position corresponding to 58, 78, 87, 155, 172, 224, 227, 272, 291, 292, or a plurality thereof, of a wild type dehalogenase. In one embodiment, the hydrolase is a mutant hydrolase such as a mutant dehalogenase having a substitution at position corresponding to 5, 11, 20, 30, 32, 47, 58, 60, 65, 78, 80, 87, 88, 94, 109, 113, 117, 118, 124, 128, 134, 136, 150, 151, 155, 157, 160, 167, 172, 175, 176, 187, 195, 204, 221, 224, 227, 231, 250, 256, 257, 263, 264, 273, 277, 282, 291 or 292, or a plurality thereof, of a wild type dehalogenase, e.g., SEQ ID NO:1. The mutant dehalogenase may thus have a plurality of substitutions including a plurality of substitutions at positions corresponding to positions 5, 11, 20, 30, 32, 47, 58, 60, 65, 78, 80, 87, 88, 94, 109, 113, 117, 118, 124, 128, 134, 136, 150, 151, 155, 157, 160, 167, 172, 187, 195, 204, 221, 224, 227, 231, 250, 256, 257, 263, 264, 277, 282, 291 or 292 of SEQ ID NO:1, at least one of which confers improved expression or binding kinetics, and may include further substitutions in positions tolerant to substitution. In one embodiment, the mutant dehaolgenase may have a plurality of substitutions including a plurality of substitutions at positions corresponding to positions 5, 7, 11, 12, 20, 30, 32, 47, 54, 55, 56, 58, 60, 65, 78, 80, 82, 87, 88, 94, 96, 109, 113, 116, 117, 118, 121, 124, 128, 131, 134, 136, 144, 147, 150, 151, 155, 157, 160, 161, 164, 165, 167, 172, 175, 176, 180, 182, 183, 187, 195, 197, 204, 218, 221, 224, 227, 231, 233, 250, 256, 257, 263, 264, 273, 277, 280, 282, 288, 291, 292, and/or 294 of SEQ ID NO:1.

Also provided is a host cell having a first polynucleotide, e.g., an expression vector, comprising an open reading frame for a first fusion protein having a first fragment of a hydrolase and a first heterologous amino acid sequence. The first fragment includes at least 20 contiguous amino acid residues of a full length hydrolase which residues are capable of associating with a second fragment of a hydrolase, which is encoded by an expression vector. The complex formed by the association of the two hydrolase fragments, but not the first hydrolase fragment or the second hydrolase fragment, is capable of stably binding a hydrolase substrate for a corresponding full length, wild type hydrolase. The N- and/or C-termini of the first and second hydrolase fragments are at a residue or in a region in a full length, wild type hydrolase sequence which is tolerant to modification. The first heterologous amino acid sequence is selected to directly or indirectly interact with a molecule of interest. In one embodiment, a host cell is provided which transiently, controllably, constitutively or stably expresses one of the polynucleotides of the invention. The second polynucleotide or its gene product may be provided via transfection, electroporation, infection, cell fusion, or any other means.

The hydrolase system of the invention may be employed to measure or detect various conditions and/or molecules of interest. For instance, protein-protein interactions are essential to virtually all aspects of cellular biology, ranging from gene transcription, protein translation, signal transduction and cell division and differentiation. Protein complementation assays (PCA) are one of several methods used to monitor protein-protein interactions. In PCA, protein-protein interactions bring two non-functional halves of an enzyme physically close to one another, which allows for re-folding into a functional enzyme. Interactions are therefore monitored by enzymatic activity. In protein complementation labeling (PCL), the detection enzyme is mutated to trap the substrate, e.g., via on acyl-mutated enzyme intermediate. Therefore, a covalent bond is created between the substrate and reconstituted mutant enzyme allowing for cumulative labeling over time, thus increasing sensitivity for the detection of weak protein-protein interactions. In one embodiment, vectors encoding two complementing fragments of a mutant dehalogenase at least one of which is fused to a protein of interest, or encoding two complementing fragments of a mutant dehalogenase each of which is fused to a protein of interest, are introduced to a cell, cell lysate, in vitro transcription/translation mixture, or supernatant, and a hydrolase substrate labeled with a functional group is added thereto. Then the functional group is detected or determined, e.g., at one or more time points and relative to a control sample. As described herein, in vitro and in vivo PCL was observed with a mutagenized dehalogenase and the protein-protein interaction system FRB/FKBP/rapamycin. Such a system uses vector constructs which allow the easy and flexible transition between in vitro and in vivo experimental systems.

In one embodiment, the invention provides a method to detect an interaction between two proteins in a sample. The method includes providing a sample having a cell comprising a plurality of expression vectors of the invention, a lysate of the cell, or an in vitro transcription/translation reaction having the plurality of expression vectors of the invention, and a hydrolase substrate with at least one functional group under conditions effective to allow for association of the first and second fusion proteins. The presence, amount or location of the at least one functional group in the sample is detected.

In another embodiment, the invention provides a method to detect a molecule of interest in a sample. The method includes providing a sample having a cell having a plurality of expression vectors of the invention, a lysate thereof, an in vitro transcription/translation reaction having the plurality of expression vectors of the invention, and a hydrolase substrate with at least one functional group under conditions effective to allow the first heterologous amino acid sequence to interact with a molecule of interest in the sample. The presence, amount or location of the at least one functional group in the sample is detected, thereby detecting the presence, amount or location of the molecule of interest.

Also provided is a method to detect an agent that alters the interaction of two proteins, which includes providing a sample having a cell comprising a plurality of expression vectors of the invention, a lysate thereof, or an in vitro transcription/translation reaction having a plurality of expression vectors of the invention, a hydrolase substrate with at least one functional group, and an agent under conditions effective to allow for association of the first and second fusion proteins. The agent is suspected of altering the interaction of the first and second heterologous amino acid sequences. The presence or amount of the at least one functional group in the sample relative to a sample without the agent is detected.

In another embodiment, the invention provides a method to detect an agent that alters the interaction of a molecule of interest and a protein. The method includes providing a sample having a cell comprising a plurality of expression vectors of the invention, a lysate thereof, or an in vitro transcription/translation reaction having the plurality of expression vectors of the invention, a hydrolase substrate with at least one functional group, and an agent suspected of altering the interaction between the heterologous amino acid sequence and a molecule of interest in the sample. The presence or amount of the functional group in the sample relative to a sample with the agent.

The invention thus provides a method of detecting the presence of a molecule of interest. For instance, a cell is contacted with vectors comprising a promoter, e.g., a regulatable promoter, and a nucleic acid sequence encoding the two complementary fragments of a mutant hydrolase, at least one of which is fused to a protein which interacts with the molecule of interest. In one embodiment, a transfected cell is cultured under conditions in which the promoter induces transient expression of the fragments or regulated expression of one of the fragments and an activity associated with the labeled substrate is detected.

### BRIEF DESCRIPTION OF THE DRAWINGS

FIG. 1A shows a molecular model of the DhaA.H272F protein. The helical cap domain is shown in light blue. The α/β hydrolase core domain (dark blue) contains the catalytic triad residues. The red shaded residues near the cap and core domain interface represent H272F and the D106 nucleophile. The yellow shaded residues denote the positions of E130 and the halide-chelating residue W107.

FIG. 1B shows the sequence of a Rhodococcus rhodochrous dehalogenase (DhaA) protein (Kulakova et al., 1997) (SEQ ID NO:1). The catalytic triad residues Asp(D), Glu(E) and His(H) are underlined. The residues that make up the cap domain are shown in italics. The DhaA.H272F and DhaA.D106C protein mutants, capable of generating covalent linkages with alkylhalide substrates, contain replacements of the catalytic triad His (H) and Asp (D) residues with Phe (F) and Cys (C), respectively.

FIG. 1C illustrates the mechanism of covalent intermediate formation by DhaA.H272F with an alkylhalide substrate. Nucleophilic displacement of the halide group by Asp106 is followed by the formation of the covalent ester intermediate. Replacement of His272 with a Phe residue prevents water activation and traps the covalent intermediate.

FIG. 1D depicts the mechanism of covalent intermediate formation by DhaA.D106C with an alkylhalide substrate. Nucleophilic displacement of the halide by the Cys106 thiolate generates a thioether intermediate that is stable to hydrolysis.

FIG. 1E depicts a structural model of the DhaA.H272F variant with a covalently attached carboxytetramethylrhodamine-C₁₀H₂NO₂—Cl ligand situated in the active site activity. The red shaded residues near the cap and core domain interface represent H272F and the D106 nucleophile. The yellow shaded residues denote the positions of E130 and the halide-chelating residue W107.

FIG. 1F shows a structural model of the DhaA.H272F substrate binding tunnel.

FIGS. 2A-B show the sequence of hits at positions 175, 176 and 273 for DhaA.H272F (panel A) and the sequence hits at positions 175 and 176 for DhaA.D106C (panel B).

FIG. 3 provides exemplary sequences of mutant dehalogenases within the scope of the invention (SEQ ID NOs:25-48). Two additional residues are encoded at the 3′ end (Gln-Tyr) as a result of cloning. Mutant dehalogenase encoding nucleic acid molecules with codons for those two additional residues are expressed at levels similar to or higher than those for mutant dehalogenases without those residues.

FIG. 4 shows the nucleotide (SEQ ID NO:17) and amino acid (SEQ ID NO:18) sequence of DhaA.H272H11YL which is in pHT2. The restriction sites listed were incorporated to facilitate generation of functional N- and C-terminal fusions.

FIG. 5 provides additional substitutions which improve functional expression of DhaA mutants with those substitutitons in E. coli.

FIG. 6 shows a schematic of protein complementation labeling (PCL).

FIG. 7 depicts an alignment of Renilla luciferase (SEQ ID NO:49) and dehalogenase sequences (SEQ ID NOs:50-51).

FIG. 8A shows a schematic of the structure of a mutant dehalogenase and exemplary sites for modificiation.

FIG. 8 B depicts expected PCL results.

FIG. 8C shows PCL results with a mutant dehalogenase.

FIG. 9 shows FluoroTect (A) and Texas Methyl Red (TMR) (B) gels of fusion proteins. M₁ (FluoroTect) from top to bottom: 155, 98, 63, 40, 32, 21, and 11 kDa. M₂ (TMR) from top to bottom: 200, 97, 66, 42, 28/20, and 14 kDa. Lane 1) full length mutant DhaA (HTv7); lane 2) FRB-HTv7 (1-78)+FKBP-HTv7 (79-297); lane 3) FRB-HTv7 (1-98)+FKBP-HTv7 (99-297); lane 4) full length Renilla luciferase (hRL); lane 5) FRB-hRL (1-91)+FKBP-hRL (92-311); lane 6) FRB-HTv7 (1-78)+FKBP-hRL (92-311); lane 7) FRB-hRL (1-91)+FKBP-HTv7 (79-297); and lane 8) no DNA. NA: not applicable to this experiment. The catalytic portion of HTv7 and Renilla luciferase reside on the respective C-terminal portion (residues 78-297 or 98-297 and residues 92-311 or 112-311, respectively).

FIG. 10 shows FluoroTect (A) and TMR (B) gels of fusion proteins. M₁ (FluoroTect and TMR) from top to bottom: 155, 98, 63, 40, 32, and 21 kDa. Lane 1) no DNA; lane 2) full length mutant DhaA (HTv7); lane 3) FRB-HTv7 (1-98)+FKBP-HTv7 (99-297); lane 4) full length Renilla luciferase (hRL); lane 5) FRB-hRL (1-111)+FKBP-hRL (112-311); lane 6) FRB-HTv7 (1-98); lane 7) FRB-hRL (1-111)+FKBP-HTv7 (99-297); lane 8) FRB-HTv7 (1-98)+FKBP-hRL (112-311); lane 9) FKBP-HTv7 (99-297); lane 10) FRB-hRL (1-111); and lane 11) FKBP-hRL (112-311).

FIGS. 11A-B depict RLU in a PCA Renilla luciferase assay.

FIG. 12 illustrates FluoroTect (A) and TMR (B) gels of fusion proteins. M₁ (FluoroTect) from top to bottom: 155, 98, 63, 40, 32, 21, and 11 kDa. M₂ (TMR) from top to bottom: 200, 97, 66, 42, 36, 28/20, and 14 kDa. Lane 1) full length mutant DhaA (HTv7); lane 2) HTv7 (1-78)-FRB+FKBP-HTv7 (79-297); lane 3) HTv7 (1-98)-FRB+FKBP-HTv7 (99-297); lane 4) full length Renilla luciferase (hRL); lane 5) hRL (1-91)-FRB+FKBP-hRL (92-311); lane 6) hRL (1-111)-FRB+FKBP-hRL (112-311); lane 7) HTv7 (1-78)-FRB+FKBP-hRL (92-311); lane 8) HTv7 (1-98)-FRB+FKBP-hRL (112-311); lane 9) hRL (1-91)-FRB+FKBP-HTv7 (79-297); lane 10) hRL (1-111)-FRB+FKBP-HTv7 (99-297); and lane 11) no DNA. Note the first lane of each sample is without rapamycin and the second lane of each sample is with rapamycin.

FIG. 13 depicts RLU for hybrid fusion proteins of the invention.

FIG. 14 provides FluoroTect (A) and TMR (B) gels of fusion proteins. M₁ (FluoroTect) from top to bottom: 155, 98, 63, 40, 32, 21, and 11 kDa. M₂ (TMR) from top to bottom: 200, 97, 66, 42, 36, 28/20, and 14 kDa. Lane 1) full length mutant DhaA (HTv7); lane 2) HTv7 (79-297)-FKBP+FRB-HTv7 (1-78); lane 3) HTv7 (99-297)-FKBP+FRB-HTv7 (1-98); lane 4) full length Renilla luciferase (hRL); lane 5) hRL (92-311)-FKBP+FRB-hRL (1-91); lane 6) hRL (112-311)-FKBP+FRB-hRL (1-111); lane 7) HTv7 (79-297)-FKBP+FRB-hRL (1-91); lane 8) HTv7 (99-297)-FKBP+FRB-hRL (1-111); lane 9) hRL (92-311)-FKBP+FRB-HTv7 (1-78); lane 10) hRL (112-311)-FKBP+FRB-HTv7 (1-98); and lane 11) no DNA.

FIG. 15 shows RLU for fusion proteins.

### DETAILED DESCRIPTION OF THE INVENTION

### Definitions

As used herein, a “substrate” includes a substrate having a reactive group and optionally one or more functional groups. A substrate which includes one or more functional groups is generally referred to herein as a substrate of the invention. A substrate, e.g., a substrate of the invention, may also optionally include a linker, e.g., a cleavable linker, which physically separates one or more functional groups from the reactive group in the substrate, and in one embodiment, the linker is preferably 12 to 30 atoms in length. The linker may not always be present in a substrate of the invention, however, in some embodiments, the physical separation of the reactive group and the functional group may be needed so that the reactive group can interact with the reactive residue in the mutant hydrolase to form a covalent bond. Preferably, when present, the linker does not substantially alter, e.g., impair, the specificity or reactivity of a substrate having the linker with the wild type or mutant hydrolase relative to the specificity or reactivity of a corresponding substrate which lacks the linker with the wild type or mutant hydrolase. Further, the presence of the linker preferably does not substantially alter, e.g., impair, one or more properties, e.g., the function, of the functional group. For instance, for some mutant hydrolases, i.e., those with deep catalytic pockets, a substrate of the invention can include a linker of sufficient length and structure so that the one or more functional groups of the substrate of the invention do not disturb the 3-D structure of the hydrolase (wild type or mutant).

As used herein, a “functional group” is a molecule which is detectable or is capable of detection, for instance, a molecule which is measurable by direct or indirect means (e.g., a photoactivatable molecule, digoxigenin, nickel NTA (nitrilotriacetic acid), a chromophore, fluorophore or luminophore), can be bound or attached to a second molecule (e.g., biotin, hapten, or a cross-linking group), or may be a solid support. A functional group may have more than one property such as being capable of detection and of being bound to another molecule.

As used herein a “reactive group” is the minimum number of atoms in a substrate which are specifically recognized by a particular wild type or mutant hydrolase of the invention. The interaction of a reactive group in a substrate and a wild type hydrolase results in a product and the regeneration of the wild type hydrolase.

As used herein, the term “heterologous” nucleic acid sequence or protein refers to a sequence that relative to a reference sequence has a different source, e.g., originates from a foreign species, or, if from the same species, it may be substantially modified from the original form.

The term “fusion polypeptide” or “fusion protein” refers to a chimeric protein containing a reference protein (e.g., a hydrolase or fragment thereof) joined at the N- and/or C-terminus to one or more heterologous sequences. In some embodiments, in the absence of an exogenous agent or molecule of interest, or under certain conditions, the heterologous sequence in a fusion polypeptide may retain at least some or have substantially the same activity as a corresponding full length (nonfused) polypeptide corresponding to the heterologous sequence. In other embodiments, in the presence of an exogenous agent or under some conditions, the heterologous sequence in a fusion polypeptide may retain at least some or have substantially the same activity as a corresponding full length (nonfused) polypeptide corresponding to the heterologous sequence.

A “nucleophile” is a molecule which donates electrons.

As used herein, a “marker gene” or “reporter gene” is a gene that imparts a distinct phenotype to cells expressing the gene and thus permits cells having the gene to be distinguished from cells that do not have the gene. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a “reporter” trait that one can identify through observation or testing, i.e., by ‘screening’. Elements of the present disclosure are exemplified in detail through the use of particular marker genes. Of course, many examples of suitable marker genes or reporter genes are known to the art and can be employed in the practice of the invention. Therefore, it will be understood that the following discussion is exemplary rather than exhaustive. In light of the techniques disclosed herein and the general recombinant techniques which are known in the art, the present invention renders possible the alteration of any gene. Exemplary modified reporter proteins are encoded by nucleic acid molecules comprising modified reporter genes including, but are not limited to, modifications of a neo gene, a □-gal gene, a gus gene, a cat gene, a gpt gene, a hyg gene, a hisD gene, a ble gene, a mprt gene, a bar gene, a nitrilase gene, a galactopyranoside gene, a xylosidase gene, a thymidine kinase gene, an arabinosidase gene, a mutant acetolactate synthase gene (ALS) or acetoacid synthase gene (AAS), a methotrexate-resistant dhfr gene, a dalapon dehalogenase gene, a mutated anthranilate synthase gene that confers resistance to 5-methyl tryptophan (WO 97/26366), an R-locus gene, a □-lactamase gene, a xylE gene, an □-amylase gene, a tyrosinase gene, a luciferase (luc) gene, (e.g., a Renilla reniformis luciferase gene, a firefly luciferase gene, or a click beetle luciferase (Pyrophorus plagiophthalamus) gene, an aequorin gene, a red fluorescent protein gene, or a green fluorescent protein gene. Included within the terms selectable or screenable marker genes are also genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA, and proteins that are inserted or trapped in the cell membrane.

A “selectable marker protein” encodes an enzymatic activity that confers to a cell the ability to grow in medium lacking what would otherwise be an essential nutrient (e.g., the TRP1 gene in yeast cells) or in a medium with an antibiotic or other drug, i.e., the expression of the gene encoding the selectable marker protein in a cell confers resistance to an antibiotic or drug to that cell relative to a corresponding cell without the gene. When a host cell must express a selectable marker to grow in selective medium, the marker is said to be a positive selectable marker (e.g., antibiotic resistance genes which confer the ability to grow in the presence of the appropriate antibiotic). Selectable markers can also be used to select against host cells containing a particular gene (e.g., the sacB gene which, if expressed, kills the bacterial host cells grown in medium containing 5% sucrose); selectable markers used in this manner are referred to as negative selectable markers or counter-selectable markers. Common selectable marker gene sequences include those for resistance to antibiotics such as ampicillin, tetracycline, kanamycin, puromycin, bleomycin, streptomycin, hygromycin, neomycin, Zeocin™, and the like. Selectable auxotrophic gene sequences include, for example, hisD, which allows growth in histidine free media in the presence of histidinol. Suitable selectable marker genes include a bleomycin-resistance gene, a metallothionein gene, a hygromycin B-phosphotransferase gene, the AURI gene, an adenosine deaminase gene, an aminoglycoside phosphotransferase gene, a dihydrofolate reductase gene, a thymidine kinase gene, a xanthine-guanine phosphoribosyltransferase gene, and the like.

A “nucleic acid”, as used herein, is a covalently linked sequence of nucleotides in which the 3□ position of the pentose of one nucleotide is joined by a phosphodiester group to the 5□ position of the pentose of the next, and in which the nucleotide residues (bases) are linked in specific sequence, i.e., a linear order of nucleotides, and includes analogs thereof, such as those having one or more modified bases, sugars and/or phosphate backbones. A “polynucleotide”, as used herein, is a nucleic acid containing a sequence that is greater than about 100 nucleotides in length. An “oligonucleotide” or “primer”, as used herein, is a short polynucleotide or a portion of a polynucleotide. The term “oligonucleotide” or “oligo” as used herein is defined as a molecule comprised of 2 or more deoxyribonucleotides or ribonucleotides, preferably more than 3, and usually more than 10, but less than 250, preferably less than 200, deoxyribonucleotides or ribonucleotides. The oligonucleotide may be generated in any manner, including chemical synthesis, DNA replication, amplification, e.g., polymerase chain reaction (PCR), reverse transcription (RT), or a combination thereof. A “primer” is an oligonucleotide which is capable of acting as a point of initiation for nucleic acid synthesis when placed under conditions in which primer extension is initiated. A primer is selected to have on its 3′ end a region that is substantially complementary to a specific sequence of the target (template). A primer must be sufficiently complementary to hybridize with a target for primer elongation to occur. A primer sequence need not reflect the exact sequence of the target. For example, a non-complementary nucleotide fragment may be attached to the 5′ end of the primer, with the remainder of the primer sequence being substantially complementary to the target. Non-complementary bases or longer sequences can be interspersed into the primer provided that the primer sequence has sufficient complementarity with the sequence of the target to hybridize and thereby form a complex for synthesis of the extension product of the primer. Primers matching or complementary to a gene sequence may be used in amplification reactions, RT-PCR and the like.

Nucleic acid molecules are said to have a “5□-terminus” (5□ end) and a “3□-terminus” (3□ end) because nucleic acid phosphodiester linkages occur to the 5□ carbon and 3□ carbon of the pentose ring of the substituent mononucleotides. The end of a polynucleotide at which a new linkage would be to a 5□ carbon is its 5□ terminal nucleotide. The end of a polynucleotide at which a new linkage would be to a 3□ carbon is its 3□ terminal nucleotide. A terminal nucleotide, as used herein, is the nucleotide at the end position of the 3□- or 5□-terminus.

DNA molecules are said to have “5□ ends” and “3□ ends” because mononucleotides are reacted to make oligonucleotides in a manner such that the 5□ phosphate of one mononucleotide pentose ring is attached to the 3□ oxygen of its neighbor in one direction via a phosphodiester linkage. Therefore, an end of an oligonucleotides referred to as the “5□ end” if its 5□ phosphate is not linked to the 3□ oxygen of a mononucleotide pentose ring and as the “3□ end” if its 3□ oxygen is not linked to a 5□ phosphate of a subsequent mononucleotide pentose ring.

As used herein, a nucleic acid sequence, even if internal to a larger oligonucleotide or polynucleotide, also may be said to have 5□ and 3□ ends. In either a linear or circular DNA molecule, discrete elements are referred to as being “upstream” or 5□ of the “downstream” or 3□ elements. This terminology reflects the fact that transcription proceeds in a 5□ to 3□ fashion along the DNA strand. Typically, promoter and enhancer elements that direct transcription of a linked gene (e.g., open reading frame or coding region) are generally located 5□ or upstream of the coding region. However, enhancer elements can exert their effect even when located 3□ of the promoter element and the coding region. Transcription termination and polyadenylation signals are located 3□ or downstream of the coding region.

The term “codon” as used herein, is a basic genetic coding unit, consisting of a sequence of three nucleotides that specify a particular amino acid to be incorporation into a polypeptide chain, or a start or stop signal. The term “coding region” when used in reference to structural gene refers to the nucleotide sequences that encode the amino acids found in the nascent polypeptide as a result of translation of a mRNA molecule. Typically, the coding region is bounded on the 5□ side by the nucleotide triplet “ATG” which encodes the initiator methionine and on the 3□ side by a stop codon (e.g., TAA, TAG, TGA). In some cases the coding region is also known to initiate by a nucleotide triplet “TTG”.

As used herein, “isolated” refers to in vitro preparation, isolation and/or purification of a nucleic acid molecule, a polypeptide, peptide or protein, so that it is not associated with in vivo substances. Thus, the term “isolated” when used in relation to a nucleic acid, as in “isolated oligonucleotide” or “isolated polynucleotide” refers to a nucleic acid sequence that is identified and separated from at least one contaminant with which it is ordinarily associated in its source. An isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. Hence, with respect to an “isolated nucleic acid molecule”, which includes a polynucleotide of genomic, cDNA, or synthetic origin or some combination thereof, the “isolated nucleic acid molecule” (1) is not associated with all or a portion of a polynucleotide in which the “isolated nucleic acid molecule” is found in nature, (2) is operably linked to a polynucleotide which it is not linked to in nature, or (3) does not occur in nature as part of a larger sequence. The isolated nucleic acid molecule may be present in single-stranded or double-stranded form. When a nucleic acid molecule is to be utilized to express a protein, the nucleic acid contains at a minimum, the sense or coding strand (i.e., the nucleic acid may be single-stranded), but may contain both the sense and anti-sense strands (i.e., the nucleic acid may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide (1) is not associated with proteins found in nature, (2) is free of other proteins from the same source, e.g., free of human proteins, (3) is expressed by a cell from a different species, or (4) does not occur in nature. Thus, an isolated polypeptide is present in a form or setting that is different from that in which it is found in nature. In contrast, non-isolated polypeptides (e.g., proteins and enzymes) are found in the state they exist in nature. The terms “isolated polypeptide”, “isolated peptide” or “isolated protein” include a polypeptide, peptide or protein encoded by cDNA or recombinant RNA including one of synthetic origin, or some combination thereof.

The term “gene” refers to a DNA sequence that comprises coding sequences and optionally control sequences necessary for the production of a polypeptide from the DNA sequence.

The term “wild type” as used herein, refers to a gene or gene product that has the characteristics of that gene or gene product isolated from a naturally occurring source. A wild type gene is that which is most frequently observed in a population and is thus arbitrarily designated the “wild type” form of the gene. In contrast, the term “mutant” refers to a gene or gene product that displays modifications in sequence and/or functional properties (i.e., altered characteristics) when compared to the wild type gene or gene product. It is noted that naturally-occurring mutants can be isolated; these are identified by the fact that they have altered characteristics when compared to the wild type gene or gene product.

Nucleic acids are known to contain different types of mutations. A “point” mutation refers to an alteration in the sequence of a nucleotide at a single base position from the wild type sequence. Mutations may also refer to insertion or deletion of one or more bases, so that the nucleic acid sequence differs from a reference, e.g., a wild type, sequence.

The term “recombinant DNA molecule” means a hybrid DNA sequence comprising at least two nucleotide sequences not normally found together in nature. The term “vector” is used in reference to nucleic acid molecules into which fragments of DNA may be inserted or cloned and can be used to transfer DNA segment(s) into a cell and capable of replication in a cell. Vectors may be derived from plasmids, bacteriophages, viruses, cosmids, and the like.

The terms “recombinant vector”, “expression vector” or “construct” as used herein refer to DNA or RNA sequences containing a desired coding sequence and appropriate DNA or RNA sequences necessary for the expression of the operably linked coding sequence in a particular host organism. Prokaryotic expression vectors include a promoter, a ribosome binding site, an origin of replication for autonomous replication in a host cell and possibly other sequences, e.g. an optional operator sequence, optional restriction enzyme sites. A promoter is defined as a DNA sequence that directs RNA polymerase to bind to DNA and to initiate RNA synthesis. Eukaryotic expression vectors include a promoter, optionally a polyadenylation signal and optionally an enhancer sequence.

A polynucleotide having a nucleotide sequence “encoding a peptide, protein or polypeptide” means a nucleic acid sequence comprising a coding region for the peptide, protein or polypeptide. The coding region may be present in either a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. In further embodiments, the coding region may contain a combination of both endogenous and exogenous control elements.

The term “transcription regulatory element” or “transcription regulatory sequence” refers to a genetic element or sequence that controls some aspect of the expression of nucleic acid sequence(s). For example, a promoter is a regulatory element that facilitates the initiation of transcription of an operably linked coding region. Other regulatory elements include, but are not limited to, transcription factor binding sites, splicing signals, polyadenylation signals, termination signals and enhancer elements, and include elements which increase or decrease transcription of linked sequences, e.g., in the presence of trans-acting elements.

Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types. For example, the SV40 early gene enhancer is very active in a wide variety of cell types from many mammalian species and has been widely used for the expression of proteins in mammalian cells. Two other examples of promoter/enhancer elements active in a broad range of mammalian cell types are those from the human elongation factor 1 gene and the long terminal repeats of the Rous sarcoma virus; and the human cytomegalovirus.

The term “promoter/enhancer” denotes a segment of DNA containing sequences capable of providing both promoter and enhancer functions (i.e., the functions provided by a promoter element and an enhancer element as described above). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques) such that transcription of the gene is directed by the linked enhancer/promoter.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook et al., 1989). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3□ end of the coding region of a given gene in the genome. A heterologous poly(A) signal is one which has been isolated from one gene and positioned 3□ to another gene. A commonly used heterologous poly(A) signal is the SV40 poly(A) signal. The SV40 poly(A) signal is contained on a 237 by BamH I/Bcl I restriction fragment and directs both termination and polyadenylation (Sambrook et al., 1989).

Eukaryotic expression vectors may also contain “viral replicons” or “viral origins of replication.” Viral replicons are viral DNA sequences which allow for the extrachromosomal replication of a vector in a host cell expressing the appropriate replication factors. Vectors containing either the SV40 or polyoma virus origin of replication replicate to high copy number (up to 10⁴ copies/cell) in cells that express the appropriate viral T antigen. In contrast, vectors containing the replicons from bovine papillomavirus or Epstein-Barr virus replicate extrachromosomally at low copy number (about 100 copies/cell).

The term “in vitro” refers to an artificial environment and to processes or reactions that occur within an artificial environment. In vitro environments include, but are not limited to, test tubes and cell lysates. The term “in situ” refers to cell culture. The term “in vivo” refers to the natural environment (e.g., an animal or a cell) and to processes or reaction that occur within a natural environment.

The term “expression system” refers to any assay or system for determining (e.g., detecting) the expression of a gene of interest. Those skilled in the field of molecular biology will understand that any of a wide variety of expression systems may be used. A wide range of suitable mammalian cells are available from a wide range of sources (e.g., the American Type Culture Collection, Rockland, Md.). The method of transformation or transfection and the choice of expression vehicle will depend on the host system selected. Transformation and transfection methods are described, e.g., in Sambrook et al., 1989. Expression systems include in vitro gene expression assays where a gene of interest (e.g., a reporter gene) is linked to a regulatory sequence and the expression of the gene is monitored following treatment with an agent that inhibits or induces expression of the gene. Detection of gene expression can be through any suitable means including, but not limited to, detection of expressed mRNA or protein (e.g., a detectable product of a reporter gene) or through a detectable change in the phenotype of a cell expressing the gene of interest. Expression systems may also comprise assays where a cleavage event or other nucleic acid or cellular change is detected.

As used herein, the terms “hybridize” and “hybridization” refer to the annealing of a complementary sequence to the target nucleic acid, i.e., the ability of two polymers of nucleic acid (polynucleotides) containing complementary sequences to anneal through base pairing. The terms “annealed” and “hybridized” are used interchangeably throughout, and are intended to encompass any specific and reproducible interaction between a complementary sequence and a target nucleic acid, including binding of regions having only partial complementarity. Certain bases not commonly found in natural nucleic acids may be included in the nucleic acids of the present invention and include, for example, inosine and 7-deazaguanine. Those skilled in the art of nucleic acid technology can determine duplex stability empirically considering a number of variables including, for example, the length of the complementary sequence, base composition and sequence of the oligonucleotide, ionic strength and incidence of mismatched base pairs. The stability of a nucleic acid duplex is measured by the melting temperature, or “Tₘ”. The Tₘ of a particular nucleic acid duplex under specified conditions is the temperature at which on average half of the base pairs have disassociated.

The term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “medium” or “low” stringency are often required when it is desired that nucleic acids which are not completely complementary to one another be hybridized or annealed together. The art knows well that numerous equivalent conditions can be employed to comprise medium or low stringency conditions. The choice of hybridization conditions is generally evident to one skilled in the art and is usually guided by the purpose of the hybridization, the type of hybridization (DNA-DNA or DNA-RNA), and the level of desired relatedness between the sequences (e.g., Sambrook et al., 1989; Nucleic Acid Hybridization, A Practical Approach, IRL Press, Washington D.C., 1985, for a general discussion of the methods).

The stability of nucleic acid duplexes is known to decrease with an increased number of mismatched bases, and further to be decreased to a greater or lesser degree depending on the relative positions of mismatches in the hybrid duplexes. Thus, the stringency of hybridization can be used to maximize or minimize stability of such duplexes. Hybridization stringency can be altered by: adjusting the temperature of hybridization; adjusting the percentage of helix destabilizing agents, such as formamide, in the hybridization mix; and adjusting the temperature and/or salt concentration of the wash solutions. For filter hybridizations, the final stringency of hybridizations often is determined by the salt concentration and/or temperature used for the post-hybridization washes.

“High stringency conditions” when used in reference to nucleic acid hybridization include conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization include conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” include conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 g/ml denatured salmon sperm DNA followed by washing in a solution comprising 5×SSPE, 0.1% SDS at 42° C. when a probe of about 500 nucleotides in length is employed.

By “peptide”, “protein” and “polypeptide” is meant any chain of amino acids, regardless of length or post-translational modification (e.g., glycosylation or phosphorylation). Unless otherwise specified, the terms are interchangeable. The nucleic acid molecules of the invention encode a fragment of a hydrolase including sequences of a variant (mutant) of a naturally-occurring (wild type) or wild type protein, which has an amino acid sequence that is substantially the same as, e.g., at least 85%, preferably 90%, and most preferably 95% or 99%, identical to the amino acid sequence of a corresponding mutant or wild type protein. The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Polypeptide molecules are said to have an “amino terminus” (N-terminus) and a “carboxy terminus” (C-terminus) because peptide linkages occur between the backbone amino group of a first amino acid residue and the backbone carboxyl group of a second amino acid residue. The terms “N-terminal” and “C-terminal” in reference to polypeptide sequences refer to regions of polypeptides including portions of the N-terminal and C-terminal regions of the polypeptide, respectively. A sequence that includes a portion of the N-terminal region of polypeptide includes amino acids predominantly from the N-terminal half of the polypeptide chain, but is not limited to such sequences. For example, an N-terminal sequence may include an interior portion of the polypeptide sequence including bases from both the N-terminal and C-terminal halves of the polypeptide. The same applies to C-terminal regions. N-terminal and C-terminal regions may, but need not, include the amino acid defining the ultimate N-terminus and C-terminus of the polypeptide, respectively.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

As used herein, the term “antibody” refers to a protein having one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies may exist as intact immunoglobulins, or as modifications in a variety of forms including, for example, FabFc₂, Fab, Fv, Fd, (Fab□)₂, an Fv fragment containing only the light and heavy chain variable regions, a Fab or (Fab)□₂ fragment containing the variable regions and parts of the constant regions, a single-chain antibody, e.g., scFv, CDR-grafted antibodies and the like. The heavy and light chain of a Fv may be derived from the same antibody or different antibodies thereby producing a chimeric Fv region. The antibody may be of animal (especially mouse or rat) or human origin or may be chimeric or humanized. As used herein the term “antibody” includes these various forms.

The terms “cell,” “cell line,” “host cell,” as used herein, are used interchangeably, and all such designations include progeny or potential progeny of these designations. By “transformed cell” is meant a cell into which (or into an ancestor of which) has been introduced a nucleic acid molecule of the invention. Optionally, a nucleic acid molecule of the invention may be introduced into a suitable cell line so as to create a stably transfected cell line capable of producing the protein or polypeptide encoded by the nucleic acid molecule. Vectors, cells, and methods for constructing such cell lines are well known in the art. The words “transformants” or “transformed cells” include the primary transformed cells derived from the originally transformed cell without regard to the number of transfers. All progeny may not be precisely identical in DNA content, due to deliberate or inadvertent mutations. Nonetheless, mutant progeny that have the same functionality as screened for in the originally transformed cell are included in the definition of transformants.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide, or a precursor thereof, e.g., the pre- or prepro-form of the protein or polypeptide, is produced.

All amino acid residues identified herein are in the natural L-configuration. In keeping with standard polypeptide nomenclature, abbreviations for amino acid residues are as shown in the following Table of Correspondence.

TABLE OF CORRESPONDENCE

| 1-Letter | 3-Letter | AMINO ACID |
|---|---|---|
| Y | Tyr | L-tyrosine |
| G | Gly | L-glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

As used herein, the term “poly-histidine tract” or (His tag) refers to a molecule comprising two to ten histidine residues, e.g., a poly-histidine tract of five to ten residues. A poly-histidine tract allows the affinity purification of a covalently linked molecule on an immobilized metal, e.g., nickel, zinc, cobalt or copper, chelate column or through an interaction with another molecule (e.g., an antibody reactive with the His tag).

The term “purified” or “to purify” means the result of any process that removes some of a contaminant from the component of interest, such as a protein or nucleic acid. The percent of a purified component is thereby increased in the sample.

As used herein, “pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a “substantially pure” composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

A “protein destabilization sequence” or “protein destabilization domain” includes one or more amino acid residues, which, when present at the N-terminus or C-terminus of a protein, reduces or decreases the half-life of the linked protein of by at least 80%, preferably at least 90%, more preferably at least 95% or more, e.g., 99%, relative to a corresponding protein which lacks the protein destabilization sequence or domain. A protein destabilization sequence includes, but is not limited to, a PEST sequence, for example, a PEST sequence from cyclin, e.g., mitotic cyclins, uracil permease or ODC, a sequence from the C-terminal region of a short-lived protein such as ODC, early response proteins such as cytokines, lymphokines, protooncogenes, e.g., c-myc or c-fos, MyoD, HMG CoA reductase, S-adenosyl methionine decarboxylase, CL sequences, a cyclin destruction box, N-degron, or a protein or a fragment thereof which is ubiquitinated in vivo.

### Hydrolases Useful to Prepare Fragments Thereof

Hydrolases within the scope of the invention include but are not limited to those prepared via recombinant techniques, e.g., site-directed mutagenesis or recursive mutagenesis, and comprise one or more amino acid substitutions which render the resulting mutant hydrolase capable of forming a stable, e.g., covalent, bond with a substrate, such as a substrate modified to contain one or more functional groups, for a corresponding nonmutant (wild type) hydrolase which bond is more stable than the bond formed between a corresponding wild type hydrolase and the substrate. Hydrolases within the scope of the invention include, but are not limited to, peptidases, esterases (e.g., cholesterol esterase), glycosidases (e.g., glucosamylase), phosphatases (e.g., alkaline phosphatase) and the like. For instance, hydrolases include, but are not limited to, enzymes acting on ester bonds such as carboxylic ester hydrolases, thiolester hydrolases, phosphoric monoester hydrolases, phosphoric diester hydrolases, triphosphoric monoester hydrolases, sulfuric ester hydrolases, diphosphoric monoester hydrolases, phosphoric triester hydrolases, exodeoxyribonucleases producing 5′-phosphomonoesters, exoribonucleases producing 5′-phosphomonoesters, exoribonucleases producing 3′-phosphomonoesters, exonucleases active with either ribo- or deoxyribonucleic acid, exonucleases active with either ribo- or deoxyribonucleic acid, endodeoxyribonucleases producing 5′-phosphomonoesters, endodeoxyribonucleases producing other than 5′-phosphomonoesters, site-specific endodeoxyribonucleases specific for altered bases, endoribonucleases producing 5′-phosphomonoesters, endoribonucleases producing other than 5′-phosphomonoesters, endoribonucleases active with either ribo- or deoxyribonucleic, endoribonucleases active with either ribo- or deoxyribonucleic glycosylases; glycosidases, e.g., enzymes hydrolyzing O- and S-glycosyl, and hydrolyzing N-glycosyl compounds; acting on ether bonds such as trialkylsulfonium hydrolases or ether hydrolases; enzymes acting on peptide bonds (peptide hydrolases) such as aminopeptidases, dipeptidases, dipeptidyl-peptidases and tripeptidyl-peptidases, peptidyl-dipeptidases, serine-type carboxypeptidases, metallocarboxypeptidases, cysteine-type carboxypeptidases, omega peptidases, serine endopeptidases, cysteine endopeptidases, aspartic endopeptidases, metalloendopeptidases, threonine endopeptidases, and endopeptidases of unknown catalytic mechanism; enzymes acting on carbon-nitrogen bonds, other than peptide bonds, such as those in linear amides, in cyclic amides, in linear amidines, in cyclic amidines, in nitriles, or other compounds; enzymes acting on acid anhydrides such as those in phosphorous-containing anhydrides and in sulfonyl-containing anhydrides; enzymes acting on acid anhydrides (catalyzing transmembrane movement); enzymes acting on acid anhydrides or involved in cellular and subcellular movement; enzymes acting on carbon-carbon bonds (e.g., in ketonic substances); enzymes acting on halide bonds (e.g., in C-halide compounds), enzymes acting on phosphorus-nitrogen bonds; enzymes acting on sulfur-nitrogen bonds; enzymes acting on carbon-phosphorus bonds; and enzymes acting on sulfur-sulfur bonds. Exemplary hydrolases acting on halide bonds include, but are not limited to, alkylhalidase, 2-haloacid dehalogenase, haloacetate dehalogenase, thyroxine deiodinase, haloalkane dehalogenase, 4-chlorobenzoate dehalogenase, 4-chlorobenzoyl-CoA dehalogenase, and atrazine chlorohydrolase. Exemplary hydrolases that act on carbon-nitrogen bonds in cyclic amides include, but are not limited to, barbiturase, dihydropyrimidinase, dihydroorotase, carboxymethylhydantoinase, allantoinase, β-lactamase, imidazolonepropionase, 5-oxoprolinase (ATP-hydrolysing), creatininase, L-lysine-lactamase, 6-aminohexanoate-cyclic-dimer hydrolase, 2,5-dioxopiperazine hydrolase, N-methylhydantoinase (ATP-hydrolysing), cyanuric acid amidohydrolase, maleimide hydrolase. “Beta-lactamase” as used herein includes Class A, Class C and Class D beta-lactamases as well as D-ala carboxypeptidase/transpeptidase, esterase EstB, penicillin binding protein 2×, penicillin binding protein 5, and D-amino peptidase. Preferably, the beta-lactamase is a serine beta-lactamase, e.g., one having a catalytic serine residue at a position corresponding to residue 70 in the serine beta-lactamase of S. aureus PC1, and a glutamic acid residue at a position corresponding to residue 166 in the serine beta-lactamase of S. aureus PC1, optionally having a lysine residue at a position corresponding to residue 73, and also optionally having a lysine residue at a position corresponding to residue 234, in the beta-lactamase of S. aureus PC1.

In one embodiment, the sequence of the mutant hydrolase formed by association of two hydrolase fragments substantially corresponds to the sequence of a mutant hydrolase having acid substitution in a residue which, in the wild type hydrolase, is associated with activating a water molecule, e.g., a residue in a catalytic triad or an auxiliary residue, wherein the activated water molecule cleaves the bond formed between a catalytic residue in the wild type hydrolase and a substrate of the hydrolase. As used herein, an “auxiliary residue” is a residue which alters the activity of another residue, e.g., it enhances the activity of a residue that activates a water molecule. Residues which activate water within the scope of the invention include but are not limited to those involved in acid-base catalysis, for instance, histidine, aspartic acid and glutamic acid. In another embodiment, the at least one amino acid substitution is in a residue which, in the wild type hydrolase, forms an ester intermediate by nucleophilic attack of a substrate for the hydrolase.

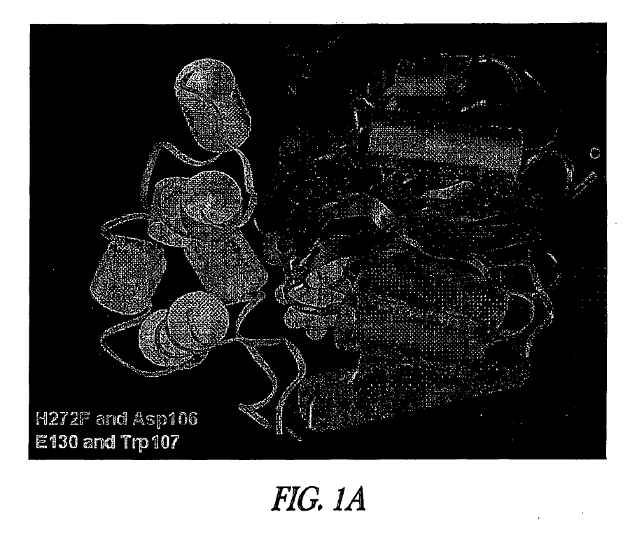

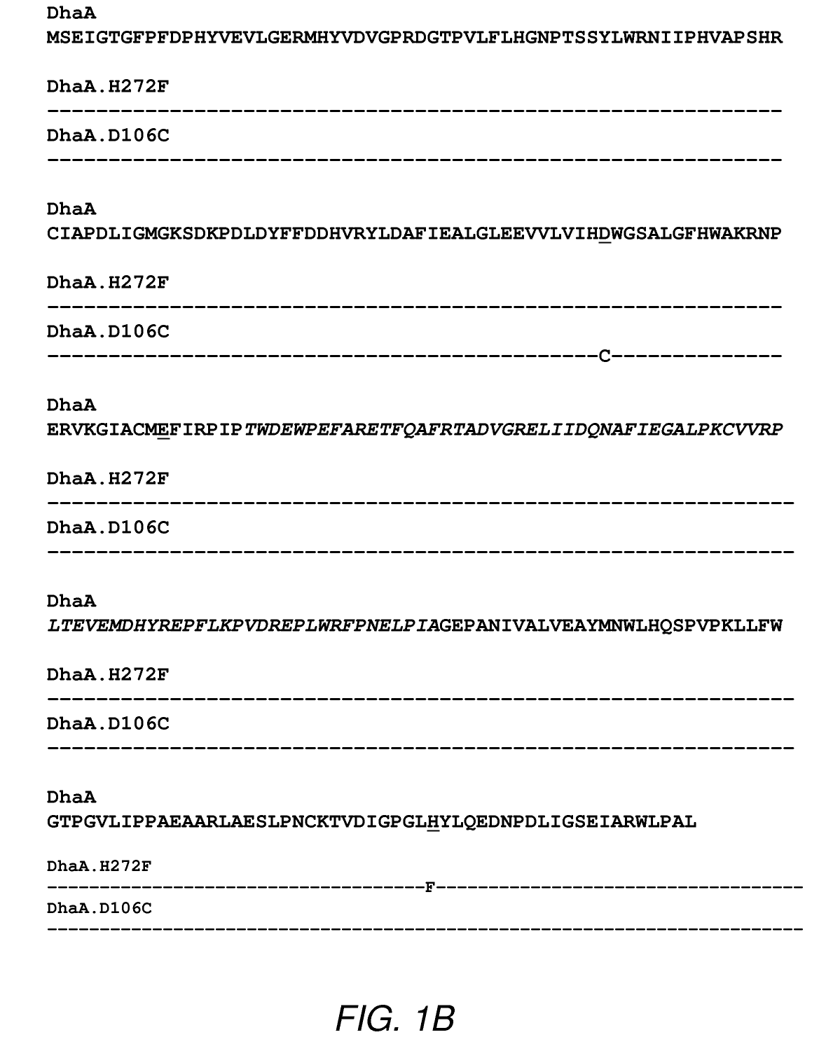

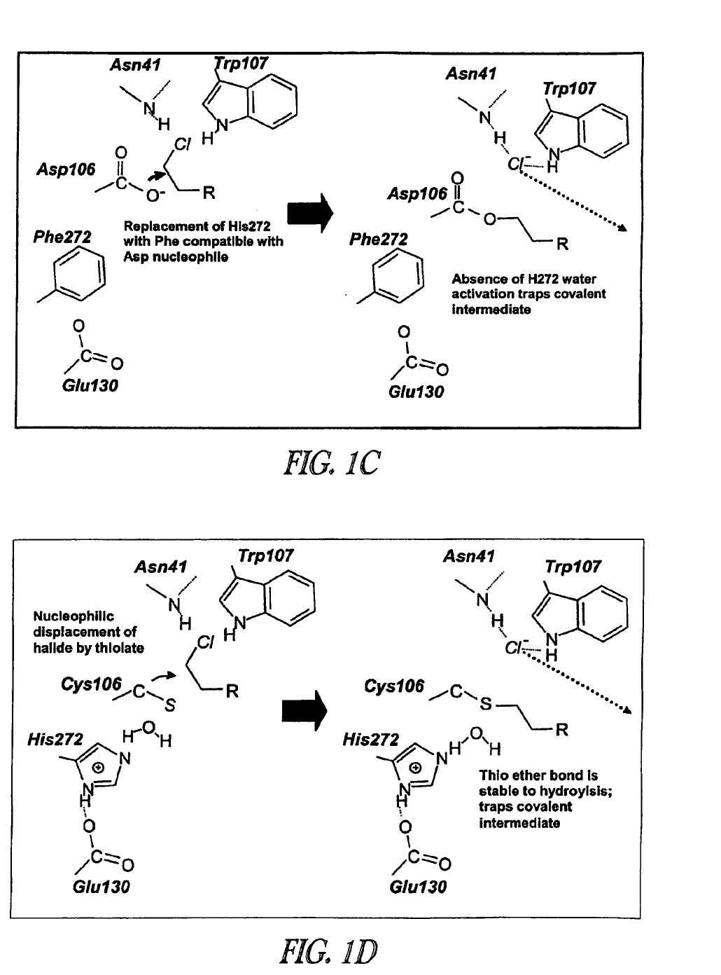

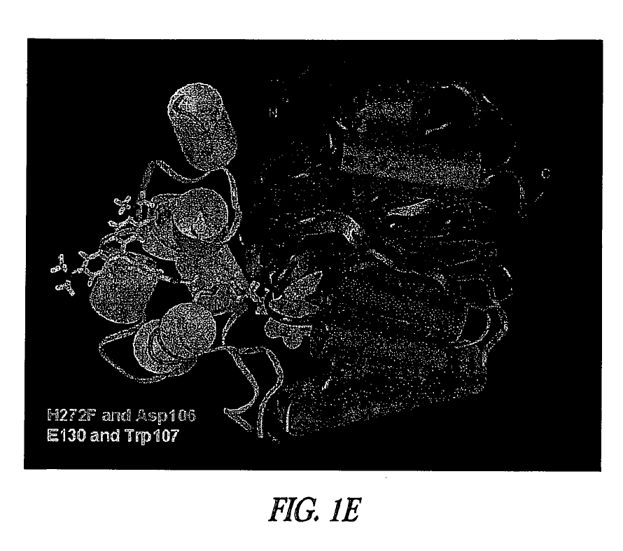

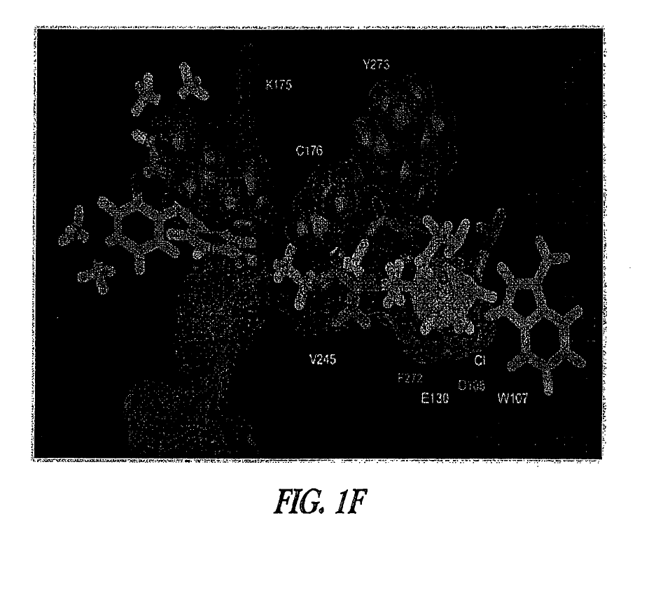

In yet another embodiment, the sequence of the mutant hydrolase formed by association of two hydrolase fragments comprises at least two amino acid substitutions, one substitution in a residue which, in the wild type hydrolase, is associated with activating a water molecule or in a residue which, in the wild type hydrolase, forms an ester intermediate by nucleophilic attack of a substrate for the hydrolase, and another substitution in a residue which, in the wild type hydrolase, is at or near a binding site(s) for a hydrolase substrate, e.g., the residue is within 3 to 5 Å of a hydrolase substrate bound to a wild type hydrolase but is not in a residue that, in the corresponding wild type hydrolase, is associated with activating a water molecule or which forms ester intermediate with a substrate. In one embodiment, the second substitution is in a residue which, in the wild type hydrolase lines the site(s) for substrate entry into the catalytic pocket of the hydrolase, e.g., a residue that is within the active site cavity and within 3 to 5 Å of a hydrolase substrate bound to the wild type hydrolase such as a residue in a tunnel for the substrate that is not a residue in the corresponding wild type hydrolase which is associated with activating a water molecule or which forms an ester intermediate with a substrate. The additional substitution(s) preferably increase the rate of stable covalent bond formation of those mutants to a substrate of a corresponding full length wild type hydrolase. In one embodiment, one substitution is at a residue in the wild type hydrolase that activates the water molecule, e.g., a histidine residue, and is at a position corresponding to amino acid residue 272 of a Rhodococcus rhodochrous dehalogenase, e.g., the substituted amino acid at the position corresponding to amino acid residue 272 is phenylalanine or glycine. In another embodiment, one substitution is at a residue in the wild type hydrolase which forms an ester intermediate with the substrate, e.g., an aspartate residue, and at a position corresponding to amino acid residue 106 of a Rhodococcus rhodochrous dehalogenase. In one embodiment, the second substitution is at an amino acid residue corresponding to a position 175, 176 or 273 of Rhodococcus rhodochrous dehalogenase, e.g., the substituted amino acid at the position corresponding to amino acid residue 175 is methionine, valine, glutamate, aspartate, alanine, leucine, serine or cysteine, the substituted amino acid at the position corresponding to amino acid residue 176 is serine, glycine, asparagine, aspartate, threonine, alanine or arginine, and/or the substituted amino acid at the position corresponding to amino acid residue 273 is leucine, methionine or cysteine. In yet another embodiment, the mutant hydrolase further comprises a third and optionally a fourth substitution at an amino acid residue in the wild type hydrolase that is within the active site cavity and within 3 to 5 Å of a hydrolase substrate bound to the wild type hydrolase, e.g., the third substitution is at a position corresponding to amino acid residue 175, 176 or 273 of a Rhodococcus rhodochrous dehalogenase, and the fourth substitution is at a position corresponding to amino acid residue 175, 176 or 273 of a Rhodococcus rhodochrous dehalogenase. In one embodiment, the mutant hydrolase of the invention comprises at least two amino acid substitutions, at least one of which is associated with stable bond formation, e.g., a residue in the wild-type hydrolase that activates the water molecule, e.g., a histidine residue, and is at a position corresponding to amino acid residue 272 of a Rhodococcus rhodochrous dehalogenase, e.g., the substituted amino acid is asparagine, glycine or phenylalanine, and at least one other is associated with improved functional expression, binding kinetics or FP signal, e.g., at a position corresponding to position 5, 11, 20, 30, 32, 47, 58, 60, 65, 78, 80, 87, 88, 94, 109, 113, 117, 118, 124, 128, 134, 136, 150, 151, 155, 157, 160, 167, 172, 187, 195, 204, 221, 224, 227, 231, 250, 256, 257, 263, 264, 277, 282, 291 or 292 of SEQ ID NO:1 (see FIG. 1B). A mutant hydrolase may include other substitution(s), e.g., those which are introduced to facilitate cloning of the corresponding gene or a portion thereof, and/or additional residue(s) at or near the N- and/or C-terminus, e.g., those which are introduced to facilitate cloning of the corresponding gene or a portion thereof but which do not necessarily have an activity, e.g., are not separately detectable.

For example, wild type dehalogenase DhaA cleaves carbon-halogen bonds in halogenated hydrocarbons (HaloC₃-HaloC₁₀). The catalytic center of DhaA is a classic catalytic triad including a nucleophile, an acid and a histidine residue. The amino acids in the triad are located deep inside the catalytic pocket of DhaA (about 10Δ long and about 20Δ² in cross section). The halogen atom in a halogenated substrate for DhaA, for instance, the chlorine atom of a Cl-alkane substrate, is positioned in close proximity to the catalytic center of DhaA. DhaA binds the substrate, likely forms an ES complex, and an ester intermediate is formed by nucleophilic attack of the substrate by Asp106 (the numbering is based on the protein sequence of DhaA) of DhaA. His272 of DhaA then activates water and the activated water hydrolyzes the intermediate, releasing product from the catalytic center. Mutant DhaAs, e.g., a DhaA.H272F mutant, which likely retains the 3-D structure based on a computer modeling study and basic physico-chemical characteristics of wild type DhaA (DhaA.WT), are not capable of hydrolyzing one or more substrates of the wild type enzyme, e.g., for Cl-alkanes, releasing the corresponding alcohol released by the wild type enzyme. Mutant serine beta-lactamases, e.g., a BlaZ.E166D mutant, a BlaZ.N170Q mutant and a BlaZ.E166D:N170Q mutant, are not capable of hydrolyzing one or more substrates of a wild type serine beta-lactamase.

Thus, in one embodiment of the invention, a mutant hydrolase formed by association of two hydrolase fragments is a mutant dehalogenase comprising at least one amino acid substitution in a residue which, in the wild type dehalogenase, is associated with activating a water molecule, e.g., a residue in a catalytic triad or an auxiliary residue, wherein the activated water molecule cleaves the bond formed between a catalytic residue in the wild type dehalogenase and a substrate of the dehalogenase. In one embodiment, at least one substitution is in a residue corresponding to residue 272 in DhaA from Rhodococcus rhodochrous. A “corresponding residue” is a residue which has the same activity (function) in one wild type protein relative to a reference wild type protein and optionally is in the same relative position when the primary sequences of the two proteins are aligned. For example, a residue which forms part of a catalytic triad and activates a water molecule in one enzyme may be residue 272 in that enzyme, which residue 272 corresponds to residue 73 in another enzyme, wherein residue 73 forms part of a catalytic triad and activates a water molecule. Thus, in one embodiment, a mutant dehalogenase has a residue other than histidine, e.g., a phenylalanine residue, at a position corresponding to residue 272 in DhaA from Rhodococcus rhodochrous. In another embodiment of the invention, a mutant hydrolase is a mutant dehalogenase comprising at least one amino acid substitution in a residue corresponding to residue 106 in DhaA from Rhodococcus rhodochrous, e.g., a substitution to a residue other than aspartate. For example, a mutant dehalogenase has a cysteine or a glutamate residue at a position corresponding to residue 106 in DhaA from Rhodococcus rhodochrous. In a further embodiment, the mutant hydrolase is a mutant dehalogenase comprising at least two amino acid substitutions, one in a residue corresponding to residue 106 and one in a residue corresponding to residue 272 in DhaA from Rhodococcus rhodochrous. In one embodiment, the mutant hydrolase is a mutant dehalogenase comprising at least two amino acid substitutions, one in a residue corresponding to residue 272 in DhaA from Rhodococcus rhodochrous and another in a residue corresponding to residue 175, 176, 245 and/or 273 in DhaA from Rhodococcus rhodochrous. In yet a further embodiment, the mutant hydrolase is a mutant serine beta-lactamase comprising at least one amino acid substitution in a residue corresponding to residue 166 or residue 170 in a serine beta-lactamase of Staphylococcus aureus PC1.

In one embodiment, the mutant hydrolase formed by association of two hydrolase fragments is a mutant haloalkane dehalogenase, e.g., such as those found in Gram-negative (Keuning et al., 1985) and Gram-positive haloalkane-utilizing bacteria (Keuning et al., 1985; Yokota et al., 1987; Scholtz et al., 1987; Sallis et al., 1990). Haloalkane dehalogenases, including Dh1A from Xanthobacter autotrophicus GJ10 (Janssen et al., 1988, 1989), DhaA from Rhodococcus rhodochrous, and LinB from Spingomonas paucimobilis UT26 (Nagata et al., 1997) are enzymes which catalyze hydrolytic dehalogenation of corresponding hydrocarbons. Halogenated aliphatic hydrocarbons subject to conversion include C₂-C₁₀ saturated aliphatic hydrocarbons which have one or more halogen groups attached, wherein at least two of the halogens are on adjacent carbon atoms. Such aliphatic hydrocarbons include volatile chlorinated aliphatic (VCA) hydrocarbons. VCA's include, for example, aliphatic hydrocarbons such as dichloroethane, 1,2-dichloro-propane, 1,2-dichlorobutane and 1,2,3-trichloropropane. The term “halogenated hydrocarbon” as used herein means a halogenated aliphatic hydrocarbon. As used herein the term “halogen” includes chlorine, bromine, iodine, fluorine, astatine and the like. A preferred halogen is chlorine.

In one embodiment, the mutant hydrolase formed by association of two hydrolase fragments is a thermostable hydrolase such as a thermostable dehalogenase comprising at least one substitution at a position corresponding to amino acid residue 117 and/or 175 of a Rhodococcus rhodochrous dehalogenase, which substitution is correlated with enhanced thermostability. In one embodiment, the thermostable hydrolase is capable of binding a hydrolase substrate at low temperatures, e.g., from 0° C. to about 25° C. In one embodiment, a thermostable hydrolase is a thermostable mutant hydrolase, i.e., one having one or more substitutions in addition to the substitution at a position corresponding to amino acid residue 117 and/or 175 of a Rhodococcus rhodochrous dehalogenase. In one embodiment, a thermostable mutant dehalogenase has a substitution which results in removal of a charged residue, e.g., lysine. In one embodiment, a thermostable mutant dehalogenase has a serine or methionine at a position corresponding to residue 117 and/or 175 in DhaA from Rhodococcus rhodochrous.

In one embodiment, the mutant hydrolase of the invention comprises at least two amino acid substitutions, at least one of which is associated with stable bond formation, e.g., a residue in the wild-type hydrolase that activates the water molecule, e.g., a histidine residue, and is at a position corresponding to amino acid residue 272 of a Rhodococcus rhodochrous dehalogenase, e.g., the substituted amino acid is asparagine, glycine or phenylalanine, and at least one other is associated with improved functional expression, binding kinetics or FP signal, e.g., at a position corresponding to position 5, 11, 20, 30, 32, 47, 58, 60, 65, 78, 80, 87, 88, 94, 109, 113, 117, 118, 124, 128, 134, 136, 150, 151, 155, 157, 160, 167, 172, 187, 195, 204, 221, 224, 227, 231, 250, 256, 257, 263, 264, 277, 282, 291 or 292 of SEQ ID NO:1.

Fusion Partners Useful with Hydrolase Fragments of the Invention

A polynucleotide of the invention which encodes a fragment of a hydrolase may be employed with other nucleic acid sequences, e.g., a native sequence such as a cDNA or one which has been manipulated in vitro, e.g., to prepare N-terminal, C-terminal, or N- and C-terminal fusion proteins. Many examples of suitable fusion partners are known to the art and can be employed in the practice of the invention.

For instance, the invention provides a fusion protein comprising a fragment of a mutant hydrolase and amino acid sequences for a protein or peptide of interest, e.g., sequences for a marker protein, e.g., a selectable marker protein, an enzyme of interest, e.g., luciferase, RNasin, RNase, and/or GFP, a nucleic acid binding protein, an extracellular matrix protein, a secreted protein, an antibody or a portion thereof such as Fc, a bioluminescence protein, a receptor ligand, a regulatory protein, a serum protein, an immunogenic protein, a fluorescent protein, a protein with reactive cysteines, a receptor protein, e.g., NMDA receptor, a channel protein, e.g., an ion channel protein such as a sodium-, potassium- or a calcium-sensitive channel protein including a HERG channel protein, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, e.g., a protease substrate, a transcription factor, a protein destabilization sequence, or a transporter protein, e.g., EAAT1-4 glutamate transporter, as well as targeting signals, e.g., a plastid targeting signal, such as a mitochondrial localization sequence, a nuclear localization signal or a myristilation sequence, that directs the mutant hydrolase to a particular location.

In one embodiment, a fusion protein includes a mutant hydrolase and a protein that is associated with a membrane or a portion thereof, e.g., targeting proteins such as those for endoplasmic reticulum targeting, cell membrane bound proteins, e.g., an integrin protein or a domain thereof such as the cytoplasmic, transmembrane and/or extracellular stalk domain of an integrin protein, and/or a protein that links the mutant hydrolase to the cell surface, e.g., a glycosylphosphoinositol signal sequence.

Fusion partners may include those having an enzymatic activity. For example, a functional protein sequence may encode a kinase catalytic domain (Hanks and Hunter, 1995), producing a fusion protein that can enzymatically add phosphate moieties to particular amino acids, or may encode a Src Homology 2 (SH2) domain (Sadowski et al., 1986; Mayer and Baltimore, 1993), producing a fusion protein that specifically binds to phosphorylated tyrosines.

The fusion may also include an affinity domain, including peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include HisV5 (HHHHH) (SEQ ID NO:13), HisX6 (HHHHHH) (SEQ ID NO:3), C-myc (EQKLISEEDL) (SEQ ID NO:4), Flag (DYKDDDDK) (SEQ ID NO:5), SteptTag (WSHPQFEK) (SEQ ID NO:6), hemagluttinin, e.g., HA Tag (YPYDVPDYA) (SEQ ID NO:7), GST, thioredoxin, cellulose binding domain, RYIRS (SEQ ID NO:8), Phe-His-His-Thr (SEQ ID NO:9), chitin binding domain, S-peptide, T7 peptide, SH2 domain, C-end RNA tag, WEAAAREACCRECCARA (SEQ ID NO:10), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D_{9K}, calbindin D_{28K}, and calretinin, inteins, biotin, streptavidin, MyoD, Id, leucine zipper sequences, and maltose binding protein.

### Optimized Hydrolase Sequences, and Vectors and Host Cells Encoding the Hydrolase

Also provided is an isolated nucleic acid molecule (polynucleotide) comprising a nucleic acid sequence encoding a hydrolase fragment or a fusion thereof. In one embodiment, the isolated nucleic acid molecule comprises a nucleic acid sequence which is optimized for expression in at least one selected host. Optimized sequences include sequences which are codon optimized, i.e., codons which are employed more frequently in one organism relative to another organism, e.g., a distantly related organism, as well as modifications to add or modify Kozak sequences and/or introns, and/or to remove undesirable sequences, for instance, potential transcription factor binding sites. In one embodiment, the polynucleotide includes a nucleic acid sequence encoding a dehalogenase, which nucleic acid sequence is optimized for expression is a selected host cell. In one embodiment, the optimized polynucleotide no longer hybridizes to the corresponding non-optimized sequence, e.g., does not hybridize to the non-optimized sequence under medium or high stringency conditions. In another embodiment, the polynucleotide has less than 90%, e.g., less than 80%, nucleic acid sequence identity to the corresponding non-optimized sequence and optionally encodes a polypeptide having at least 80%, e.g., at least 85%, 90% or more, amino acid sequence identity with the polypeptide encoded by the non-optimized sequence. Constructs, e.g., expression cassettes, and vectors comprising the isolated nucleic acid molecule, as well as kits comprising the isolated nucleic acid molecule, construct or vector are also provided.

A nucleic acid molecule comprising a nucleic acid sequence encoding a hydrolase fragment or a fusion with a hydrolase fragment is optionally optimized for expression in a particular host cell and also optionally operably linked to transcription regulatory sequences, e.g., one or more enhancers, a promoter, a transcription termination sequence or a combination thereof, to form an expression cassette.

In one embodiment, a nucleic acid sequence encoding a hydrolase fragment or a fusion thereof is optimized by replacing codons in a wild type or mutant hydrolase sequence with codons which are preferentially employed in a particular (selected) cell. Preferred codons have a relatively high codon usage frequency in a selected cell, and preferably their introduction results in the introduction of relatively few transcription factor binding sites for transcription factors present in the selected host cell, and relatively few other undesirable structural attributes. Thus, the optimized nucleic acid product has an improved level of expression due to improved codon usage frequency, and a reduced risk of inappropriate transcriptional behavior due to a reduced number of undesirable transcription regulatory sequences.

An isolated and optimized nucleic acid molecule of the invention may have a codon composition that differs from that of the corresponding wild type nucleic acid sequence at more than 30%, 35%, 40% or more than 45%, e.g., 50%, 55%, 60% or more of the codons. Preferred codons for use in the invention are those which are employed more frequently than at least one other codon for the same amino acid in a particular organism and, more preferably, are also not low-usage codons in that organism and are not low-usage codons in the organism used to clone or screen for the expression of the nucleic acid molecule. Moreover, preferred codons for certain amino acids (i.e., those amino acids that have three or more codons), may include two or more codons that are employed more frequently than the other (non-preferred) codon(s). The presence of codons in the nucleic acid molecule that are employed more frequently in one organism than in another organism results in a nucleic acid molecule which, when introduced into the cells of the organism that employs those codons more frequently, is expressed in those cells at a level that is greater than the expression of the wild type or parent nucleic acid sequence in those cells.

In one embodiment of the invention, the codons that are different are those employed more frequently in a mammal, while in another embodiment the codons that are different are those employed more frequently in a plant. Preferred codons for different organisms are known to the art, e.g., see www.kazusa.or.jp./codon/. A particular type of mammal, e.g., a human, may have a different set of preferred codons than another type of mammal. Likewise, a particular type of plant may have a different set of preferred codons than another type of plant. In one embodiment of the invention, the majority of the codons that differ are ones that are preferred codons in a desired host cell. Preferred codons for organisms including mammals (e.g., humans) and plants are known to the art (e.g., Wada et al., 1990; Ausubel et al., 1997). For example, preferred human codons include, but are not limited to, CGC (Arg), CTG (Leu), TCT (Ser), AGC (Ser), ACC (Thr), CCA (Pro), CCT (Pro), GCC (Ala), GGC (Gly), GTG (Val), ATC (Ile), ATT (Ile), AAG (Lys), AAC (Asn), CAG (Gln), CAC (His), GAG (Glu), GAC (Asp), TAC (Tyr), TGC (Cys) and TTC (Phe) (Wada et al., 1990). Thus, in one embodiment, synthetic nucleic acid molecules of the invention have a codon composition which differs from a wild type nucleic acid sequence by having an increased number of the preferred human codons, e.g., CGC, CTG, TCT, AGC, ACC, CCA, CCT, GCC, GGC, GTG, ATC, ATT, AAG, AAC, CAG, CAC, GAG, GAC, TAC, TGC, TTC, or any combination thereof. For example, the nucleic acid molecule of the invention may have an increased number of CTG or TTG leucine-encoding codons, GTG or GTC valine-encoding codons, GGC or GGT glycine-encoding codons, ATC or ATT isoleucine-encoding codons, CCA or CCT proline-encoding codons, CGC or CGT arginine-encoding codons, AGC or TCT serine-encoding codons, ACC or ACT threonine-encoding codon, GCC or GCT alanine-encoding codons, or any combination thereof, relative to the wild type nucleic acid sequence. In another embodiment, preferred C. elegans codons include, but are not limited, to UUC (Phe), UUU (Phe), CUU (Leu), UUG (Leu), AUU (Ile), GUU (Val), GUG (Val), UCA (Ser), UCU (Ser), CCA (Pro), ACA (Thr), ACU (Thr), GCU (Ala), GCA (Ala), UAU (Tyr), CAU (His), CAA (Gln), AAU (Asn), AAA (Lys), GAU (Asp), GAA (Glu), UGU (Cys), AGA (Arg), CGA (Arg), CGU (Arg), GGA (Gly), or any combination thereof. In yet another embodiment, preferred Drosophilia codons include, but are not limited to, UUC (Phe), CUG (Leu), CUC (Leu), AUC (Ile), AUU (Ile), GUG (Val), GUC (Val), AGC (Ser), UCC (Ser), CCC (Pro), CCG (Pro), ACC (Thr), ACG (Thr), GCC (Ala), GCU (Ala), UAC (Tyr), CAC (His), CAG (Gln), AAC (Asn), AAG (Lys), GAU (Asp), GAG (Glu), UGC (Cys), CGC (Arg), GGC (Gly), GGA (gly), or any combination thereof. Preferred yeast codons include but are not limited to UUU (Phe), UUG (Leu), UUA (Leu), CCU (Leu), AUU (Ile), GUU (Val), UCU (Ser), UCA (Ser), CCA (Pro), CCU (Pro), ACU (Thr), ACA (Thr), GCU (Ala), GCA (Ala), UAU (Tyr), UAC (Tyr), CAU (His), CAA (Gln), AAU (Asn), AAC (Asn), AAA (Lys), AAG (Lys), GAU (Asp), GAA (Glu), GAG (Glu), UGU (Cys), CGU (Trp), AGA (Arg), CGU (Arg), GGU (Gly), GGA (Gly), or any combination thereof. Similarly, nucleic acid molecules having an increased number of codons that are employed more frequently in plants, have a codon composition which differs from a wild type or parent nucleic acid sequence by having an increased number of the plant codons including, but not limited to, CGC (Arg), CTT (Leu), TCT (Ser), TCC (Ser), ACC (Thr), CCA (Pro), CCT (Pro), GCT (Ser), GGA (Gly), GTG (Val), ATC (Ile), ATT (Ile), AAG (Lys), AAC (Asn), CAA (Gln), CAC (His), GAG (Glu), GAC (Asp), TAC (Tyr), TGC (Cys), TTC (Phe), or any combination thereof (Murray et al., 1989). Preferred codons may differ for different types of plants (Wada et al., 1990).

In one embodiment, an optimized nucleic acid sequence encoding a hydrolase fragment or fusion thereof has less than 100%, e.g., less than 90% or less than 80%, nucleic acid sequence identity relative to a non-optimized nucleic acid sequence encoding a corresponding hydrolase fragment or fusion thereof. For instance, an optimized nucleic acid sequence encoding DhaA has less than about 80% nucleic acid sequence identity relative to non-optimized (wild type) nucleic acid sequence encoding a corresponding DhaA, and the DhaA encoded by the optimized nucleic acid sequence optionally has at least 85% amino acid sequence identity to a corresponding wild type DhaA. In one embodiment, the activity of a DhaA encoded by the optimized nucleic acid sequence is at least 10%, e.g., 50% or more, of the activity of a DhaA encoded by the non-optimized sequence, e.g., a mutant DhaA encoded by the optimized nucleic acid sequence binds a substrate with substantially the same efficiency, i.e., at least 50%, 80%, 100% or more, as the mutant DhaA encoded by the non-optimized nucleic acid sequence binds the same substrate.

An exemplary optimized DhaA gene has the following sequence:

hDhaA.v2.1-6F (FINAL, with flanking sequences)
(SEQ ID NO: 16)
NNNNGCTAGCCAGCTGGCgcgGATATCGCCACCATGGGATCCGAGATT
GGGACAGGGTTcCCTTTTGATCCTCAcTATGTtGAaGTGCTGGGgGAa
AGAATGCAcTAcGTGGATGTGGGGCCTAGAGATGGGACcCCaGTGCTG
TTcCTcCAcGGGAAcCCTACATCTagcTAcCTGTGGAGaAAtATTATa
CCTCATGTtGCTCCTagtCATAGgTGcATTGCTCCTGATCTGATcGGG
ATGGGGAAGTCTGATAAGCCTGActtaGAcTAcTTTTTTGATGAtCAT
GTtcGATActTGGATGCTTTcATTGAGGCTCTGGGGCTGGAGGAGGTG
GTGCTGGTGATaCAcGAcTGGGGGTCTGCTCTGGGGTTTCAcTGGGCT
AAaAGgAATCCgGAGAGAGTGAAGGGGATTGCTTGcATGGAgTTTATT
cGACCTATTCCTACtTGGGAtGAaTGGCCaGAGTTTGCcAGAGAGACA
TTTCAaGCcTTTAGAACtGCcGATGTGGGcAGgGAGCTGATTATaGAc
CAGAATGCTTTcATcGAGGGGGCTCTGCCTAAaTGTGTaGTcAGACCT
CTcACtGAaGTaGAGATGGAcCATTATAGAGAGCCcTTTCTGAAGCCT
GTGGATcGcGAGCCTCTGTGGAGgTTtCCaAATGAGCTGCCTATTGCT
GGGGAGCCTGCTAATATTGTGGCTCTGGTGGAaGCcTATATGAAcTGG
CTGCATCAGagTCCaGTGCCcAAGCTaCTcTTTTGGGGGACtCCgGGa
GTtCTGATTCCTCCTGCcGAGGCTGCTAGACTGGCTGAaTCcCTGCCc
AAtTGTAAGACcGTGGAcATcGGcCCtGGgCTGTTTTAcCTcCAaGAG
GAcAAcCCTGATCTcATcGGGTCTGAGATcGCacGgTGGCTGCCCGGG
CTGGCCGGCTAATAGTTAATTAAGTAgGCGGCCGCNNNN.

The nucleic acid molecule or expression cassette may be introduced to a vector, e.g., a plasmid or viral vector, which optionally includes a selectable marker gene, and the vector introduced to a cell of interest, for example, a prokaryotic cell such as E. coli, Streptomyces spp., Bacillus spp., Staphylococcus spp. and the like, as well as eukaryotic cells including a plant (dicot or monocot), fungus, yeast, e.g., Pichia, Saccharomyces or Schizosaccharomyces, or mammalian cell. Preferred mammalian cells include bovine, caprine, ovine, canine, feline, non-human primate, e.g., simian, and human cells. Preferred mammalian cell lines include, but are not limited to, CHO, COS, 293, Hela, CV-1, SH-SY5Y (human neuroblastoma cells), HEK293, and NIH3T3 cells.

The expression of the encoded hydrolase fragment may be controlled by any promoter capable of expression in prokaryotic cells or eukaryotic cells. Preferred prokaryotic promoters include, but are not limited to, SP6, T7, T5, tac, bla, trp, gal, lac or maltose promoters. Preferred eukaryotic promoters include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Preferred vectors for bacterial expression include pGEX-5X-3, and for eukaryotic expression include pClneo-CMV.

The nucleic acid molecule, expression cassette and/or vector of the invention may be introduced to a cell by any method including, but not limited to, calcium-mediated transformation, electroporation, microinjection, lipofection, particle bombardment and the like.

### Functional Groups

Functional groups useful in the substrates and methods of the invention are molecules that are detectable or capable of detection. A functional group within the scope of the invention is capable of being covalently linked to one reactive substituent of a bifunctional linker or a substrate for a hydrolase, and, as part of a substrate of the invention, has substantially the same activity as a functional group which is not linked to a substrate found in nature and is capable of forming a stable complex with a mutant hydrolase. Functional groups thus have one or more properties that facilitate detection, and optionally the isolation, of stable complexes between a substrate having that functional group and a mutant hydrolase. For instance, functional groups include those with a characteristic electromagnetic spectral property such as emission or absorbance, magnetism, electron spin resonance, electrical capacitance, dielectric constant or electrical conductivity as well as functional groups which are ferromagnetic, paramagnetic, diamagnetic, luminescent, electrochemiluminescent, fluorescent, phosphorescent, chromatic, antigenic, or have a distinctive mass. A functional group includes, but is not limited to, a nucleic acid molecule, i.e., DNA or RNA, e.g., an oligonucleotide or nucleotide, such as one having nucleotide analogs, DNA which is capable of binding a protein, single stranded DNA corresponding to a gene of interest, RNA corresponding to a gene of interest, mRNA which lacks a stop codon, an aminoacylated initiator tRNA, an aminoacylated amber suppressor tRNA, or double stranded RNA for RNAi, a protein, e.g., a luminescent protein, a peptide, a peptide nucleic acid, an epitope recognized by a ligand, e.g., biotin or streptavidin, a hapten, an amino acid, a lipid, a lipid bilayer, a solid support, a fluorophore, a chromophore, a reporter molecule, a radionuclide, such as a radioisotope for use in, for instance, radioactive measurements or a stable isotope for use in methods such as isotope coded affinity tag (ICAT), an electron opaque molecule, an X-ray contrast reagent, a MRI contrast agent, e.g., manganese, gadolinium (III) or iron-oxide particles, and the like. In one embodiment, the functional group is an amino acid, protein, glycoprotein, polysaccharide, triplet sensitizer, e.g., CALI, nucleic acid molecule, drug, toxin, lipid, biotin, or solid support, such as self-assembled monolayers (see, e.g., Kwon et al., 2004), binds Ca²⁺, binds K⁺, binds Na⁺, is pH sensitive, is electron opaque, is a chromophore, is a MRI contrast agent, fluoresces in the presence of NO or is sensitive to a reactive oxygen, a nanoparticle, an enzyme, a substrate for an enzyme, an inhibitor of an enzyme, for instance, a suicide substrate (see, e.g., Kwon et al., 2004), a cofactor, e.g., NADP, a coenzyme, a succinimidyl ester or aldehyde, luciferin, glutathione, NTA, biotin, cAMP, phosphatidylinositol, a ligand for cAMP, a metal, a nitroxide or nitrone for use as a spin trap (detected by electron spin resonance (ESR), a metal chelator, e.g., for use as a contrast agent, in time resolved fluorescence or to capture metals, a photocaged compound, e.g., where irradiation liberates the caged compound such as a fluorophore, an intercalator, e.g., such as psoralen or another intercalator useful to bind DNA or as a photoactivatable molecule, a triphosphate or a phosphoramidite, e.g., to allow for incorporation of the substrate into DNA or RNA, an antibody, or a heterobifunctional cross-linker such as one useful to conjugate proteins or other molecules, cross-linkers including but not limited to hydrazide, aryl azide, maleimide, iodoacetamide/bromoacetamide, N-hydroxysuccinimidyl ester, mixed disulfide such as pyridyl disulfide, glyoxal/phenylglyoxal, vinyl sulfone/vinyl sulfonamide, acrylamide, boronic ester, hydroxamic acid, imidate ester, isocyanate/isothiocyanate, or chlorotriazine/dichlorotriazine.

For instance, a functional group includes but is not limited to one or more amino acids, e.g., a naturally occurring amino acid or a non-natural amino acid, a peptide or polypeptide (protein) including an antibody or a fragment thereof, a His-tag, a FLAG tag, a Strep-tag, an enzyme, a cofactor, a coenzyme, a peptide or protein substrate for an enzyme, for instance, a branched peptide substrate (e.g., Z-aminobenzoyl (Abz)-Gly-Pro-Ala-Leu-Ala-4-nitrobenzyl amide (NBA) (SEQ ID NO:20 represents Gly-Pro-Ala-Leu-Ala), a suicide substrate, or a receptor, one or more nucleotides (e.g., ATP, ADP, AMP, GTP or GDP) including analogs thereof, e.g., an oligonucleotide, double stranded or single stranded DNA corresponding to a gene or a portion thereof, e.g., DNA capable of binding a protein such as a transcription factor, RNA corresponding to a gene, for instance, mRNA which lacks a stop codon, or a portion thereof, double stranded RNA for RNAi or vectors therefor, a glycoprotein, a polysaccharide, a peptide-nucleic acid (PNA), lipids including lipid bilayers; or is a solid support, e.g., a sedimental particle such as a magnetic particle, a sepharose or cellulose bead, a membrane, glass, e.g., glass slides, cellulose, alginate, plastic or other synthetically prepared polymer, e.g., an eppendorf tube or a well of a multi-well plate, self assembled monolayers, a surface plasmon resonance chip, or a solid support with an electron conducting surface, and includes a drug, for instance, a chemotherapeutic such as doxorubicin, 5-fluorouracil, or camptosar (CPT-11; Irinotecan), an aminoacylated tRNA such as an aminoacylated initiator tRNA or an aminoacylated amber suppressor tRNA, a molecule which binds Ca²⁺, a molecule which binds K⁺, a molecule which binds Na⁺, a molecule which is pH sensitive, a radionuclide, a molecule which is electron opaque, a contrast agent, e.g., barium, iodine or other MRI or X-ray contrast agent, a molecule which fluoresces in the presence of NO or is sensitive to a reactive oxygen, a nanoparticle, e.g., an immunogold particle, paramagnetic nanoparticle, upconverting nanoparticle, or a quantum dot, a nonprotein substrate for an enzyme, an inhibitor of an enzyme, either a reversible or irreversible inhibitor, a chelating agent, a cross-linking group, for example, a succinimidyl ester or aldehyde, glutathione, biotin or other avidin binding molecule, avidin, streptavidin, cAMP, phosphatidylinositol, heme, a ligand for cAMP, a metal, NTA, and, in one embodiment, includes one or more dyes, e.g., a xanthene dye, a calcium sensitive dye, e.g., 1-[2-amino-5-(2,7-dichloro-6-hydroxy-3-oxy-9-xanthenyl)-phenoxy]-2-(2′-amino-5′-methylphenoxy)ethane-N,N,N′,N′-tetraacetic acid (Fluo-3), a sodium sensitive dye, e.g., 1,3-benzenedicarboxylic acid, 4,4′-[1,4,10,13-tetraoxa-7,16-diazacyclooctadecane-7,16-diylbis(5-methoxy-6,2-benzofurandiyl)]bis(PBFI), a NO sensitive dye, e.g., 4-amino-5-methylamino-2′,7′-difluorescein, or other fluorophore. In one embodiment, the functional group is a hapten or an immunogenic molecule, i.e., one which is bound by antibodies specific for that molecule. In one embodiment, the functional group is not a radionuclide. In another embodiment, the functional group is a radionuclide, e.g., 3H, ¹⁴C, ³⁵S, ¹²⁵I, ¹³¹I, including a molecule useful in diagnostic methods.

Methods to detect a particular functional group are known to the art. For example, a nucleic acid molecule can be detected by hybridization, amplification, binding to a nucleic acid binding protein specific for the nucleic acid molecule, enzymatic assays (e.g., if the nucleic acid molecule is a ribozyme), or, if the nucleic acid molecule itself comprises a molecule which is detectable or capable of detection, for instance, a radiolabel or biotin, it can be detected by an assay suitable for that molecule.

Exemplary functional groups include haptens, e.g., molecules useful to enhance immunogenicity such as keyhole limpet hemacyanin (KLH), cleavable labels, for instance, photocleavable biotin, and fluorescent labels, e.g., N-hydroxysuccinimide (NHS) modified coumarin and succinimide or sulfonosuccinimide modified BODIPY (which can be detected by UV and/or visible excited fluorescence detection), rhodamine, e.g., R110, rhodols, CRG6, Texas Methyl Red (carboxytetramethylrhodamine), 5-carboxy-X-rhodamine, or fluoroscein, coumarin derivatives, e.g., 7 aminocoumarin, and 7-hydroxycoumarin, 2-amino-4-methoxynapthalene, 1-hydroxypyrene, resorufin, phenalenones or benzphenalenones (U.S. Pat. No. 4,812,409), acridinones (U.S. Pat. No. 4,810,636), anthracenes, and derivatives of α- and β-napthol, fluorinated xanthene derivatives including fluorinated fluoresceins and rhodols (e.g., U.S. Pat. No. 6,162,931), bioluminescent molecules, e.g., luciferin, coelenterazine, luciferase, chemiluminescent molecules, e.g., stabilized dioxetanes, and electrochemiluminescent molecules. A fluorescent (or luminescent) functional group linked to a mutant hydrolase by virtue of being linked to a substrate for a corresponding wild type hydrolase, may be used to sense changes in a system, like phosphorylation, in real time. Moreover, a fluorescent molecule, such as a chemosensor of metal ions, e.g., a 9-carbonylanthracene modified glycyl-histidyl-lysine (GHK) for Cu²⁺, in a substrate of the invention may be employed to label proteins which bind the substrate. A luminescent or fluorescent functional group such as BODIPY, rhodamine green, GFP, or infrared dyes, also finds use as a functional group and may, for instance, be employed in interaction studies, e.g., using BRET, FRET, LRET or electrophoresis.

Another class of functional group is a molecule that selectively interacts with molecules containing acceptor groups (an “affinity” molecule). Thus, a substrate for a hydrolase which includes an affinity molecule can facilitate the separation of complexes having such a substrate and a mutant hydrolase, because of the selective interaction of the affinity molecule with another molecule, e.g., an acceptor molecule, that may be biological or non-biological in origin. For example, the specific molecule with which the affinity molecule interacts (referred to as the acceptor molecule) could be a small organic molecule, a chemical group such as a sulfhydryl group (—SH) or a large biomolecule such as an antibody or other naturally occurring ligand for the affinity molecule. The binding is normally chemical in nature and may involve the formation of covalent or non-covalent bonds or interactions such as ionic or hydrogen bonding. The acceptor molecule might be free in solution or itself bound to a solid or semi-solid surface, a polymer matrix, or reside on the surface of a solid or semi-solid substrate. The interaction may also be triggered by an external agent such as light, temperature, pressure or the addition of a chemical or biological molecule that acts as a catalyst. The detection and/or separation of the complex from the reaction mixture occurs because of the interaction, normally a type of binding, between the affinity molecule and the acceptor molecule.

Examples of affinity molecules include molecules such as immunogenic molecules, e.g., epitopes of proteins, peptides, carbohydrates or lipids, i.e., any molecule which is useful to prepare antibodies specific for that molecule; biotin, avidin, streptavidin, and derivatives thereof; metal binding molecules; and fragments and combinations of these molecules. Exemplary affinity molecules include His5 (HHHHH) (SEQ ID NO:13), His X6 (HHHHHH) (SEQ ID NO:3), C-myc (EQKLISEEDL) (SEQ ID NO:4), Flag (DYKDDDDK) (SEQ ID NO:5), SteptTag (WSHPQFEK) (SEQ ID NO:6), HA Tag (YPYDVPDYA) (SEQ ID NO:7), thioredoxin, cellulose binding domain, chitin binding domain, S-peptide, T7 peptide, calmodulin binding peptide, C-end RNA tag, metal binding domains, metal binding reactive groups, amino acid reactive groups, inteins, biotin, streptavidin, and maltose binding protein. The presence of the biotin in a complex between the mutant hydrolase and the substrate permits selective binding of the complex to avidin molecules, e.g., streptavidin molecules coated onto a surface, e.g., beads, microwells, nitrocellulose and the like. Suitable surfaces include resins for chromatographic separation, plastics such as tissue culture surfaces or binding plates, microtiter dishes and beads, ceramics and glasses, particles including magnetic particles, polymers and other matrices. The treated surface is washed with, for example, phosphate buffered saline (PBS), to remove molecules that lack biotin and the biotin-containing complexes isolated. In some case these materials may be part of biomolecular sensing devices such as optical fibers, chemfets, and plasmon detectors.

Another example of an affinity molecule is dansyllysine. Antibodies which interact with the dansyl ring are commercially available (Sigma Chemical; St. Louis, Mo.) or can be prepared using known protocols such as described in Antibodies: A Laboratory Manual (Harlow and Lane, 1988). For example, the anti-dansyl antibody is immobilized onto the packing material of a chromatographic column. This method, affinity column chromatography, accomplishes separation by causing the complex between a mutant hydrolase and a substrate of the invention to be retained on the column due to its interaction with the immobilized antibody, while other molecules pass through the column. The complex may then be released by disrupting the antibody-antigen interaction. Specific chromatographic column materials such as ion-exchange or affinity Sepharose, Sephacryl, Sephadex and other chromatography resins are commercially available (Sigma Chemical; St. Louis, Mo.; Pharmacia Biotech; Piscataway, N.J.). Dansyllysine may conveniently be detected because of its fluorescent properties.

When employing an antibody as an acceptor molecule, separation can also be performed through other biochemical separation methods such as immunoprecipitation and immobilization of antibodies on filters or other surfaces such as beads, plates or resins. For example, complexes of a mutant hydrolase and a substrate of the invention may be isolated by coating magnetic beads with an affinity molecule-specific or a hydrolase-specific antibody. Beads are oftentimes separated from the mixture using magnetic fields.

Another class of functional molecules includes molecules detectable using electromagnetic radiation and includes but is not limited to xanthene fluorophores, dansyl fluorophores, coumarins and coumarin derivatives, fluorescent acridinium moieties, benzopyrene based fluorophores, as well as 7-nitrobenz-2-oxa-1,3-diazole, and 3-N-(7-nitrobenz-2-oxa-1,3-diazol-4-yl)-2,3-diamino-propionic acid. Preferably, the fluorescent molecule has a high quantum yield of fluorescence at a wavelength different from native amino acids and more preferably has high quantum yield of fluorescence that can be excited in the visible, or in both the UV and visible, portion of the spectrum. Upon excitation at a preselected wavelength, the molecule is detectable at low concentrations either visually or using conventional fluorescence detection methods. Electrochemiluminescent molecules such as ruthenium chelates and its derivatives or nitroxide amino acids and their derivatives are detectable at femtomolar ranges and below.

In one embodiment, an optically detectable functional group includes one or more fluorophores, such as a xanthene, coumarin, chromene, indole, isoindole, oxazole, BODIPY, a BODIPY derivative, imidazole, pyrimidine, thiophene, pyrene, benzopyrene, benzofuran, fluorescein, rhodamine, rhodol, phenalenone, acridinone, resorufin, naphthalene, anthracene, acridinium, α-napthol, β-napthol, dansyl, cyanines, oxazines, nitrobenzoxazole (NBD), dapoxyl, naphthalene imides, styryls, and the like.

In one embodiment, an optically detectable functional group includes one of:

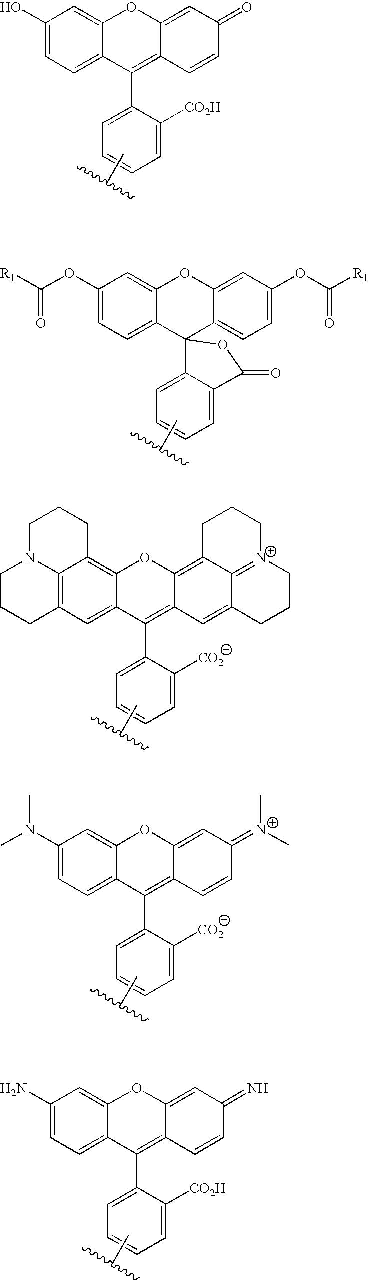

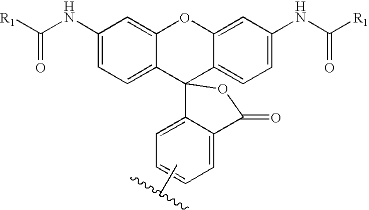

wherein R₁ is C₁-C₈.

In addition to fluorescent molecules, a variety of molecules with physical properties based on the interaction and response of the molecule to electromagnetic fields and radiation can be used to detect complexes between a mutant hydrolase or fragment thereof and a substrate. These properties include absorption in the UV, visible and infrared regions of the electromagnetic spectrum, presence of chromophores which are Raman active, and can be further enhanced by resonance Raman spectroscopy, electron spin resonance activity and nuclear magnetic resonances and molecular mass, e.g., via a mass spectrometer.

Methods to detect and/or isolate complexes having affinity molecules include chromatographic techniques including gel filtration, fast-pressure or high-pressure liquid chromatography, reverse-phase chromatography, affinity chromatography and ion exchange chromatography. Other methods of protein separation are also useful for detection and subsequent isolation of complexes between a mutant hydrolase or a fragment thereof and a substrate, for example, electrophoresis, isoelectric focusing and mass spectrometry.

### Linkers

The term “linker”, which is also identified by the symbol >L=, refers to a group or groups that covalently attach one or more functional groups to a substrate which includes a reactive group or to a reactive group. A linker, as used herein, is not a single covalent bond. The structure of the linker is not crucial, provided it yields a substrate that can be bound by its target enzyme. In one embodiment, the linker can be a divalent group that separates a functional group (R) and the reactive group by about 5 angstroms to about 1000 angstroms, inclusive, in length. Other suitable linkers include linkers that separate R and the reactive group by about 5 angstroms to about 100 angstroms, as well as linkers that separate R and the substrate by about 5 angstroms to about 50 angstroms, by about 5 angstroms to about 25 angstroms, by about 5 angstroms to about 500 angstroms, or by about 30 angstroms to about 100 angstroms.

In one embodiment the linker is an amino acid.

In another embodiment, the linker is a peptide.

In another embodiment, the linker is a divalent branched or unbranched carbon chain comprising from about 2 to about 30 carbon atoms, which chain optionally includes one or more (e.g., 1, 2, 3, or 4) double or triple bonds, and which chain is optionally substituted with one or more (e.g., 2, 3, or 4) hydroxy or oxo (═O) groups, wherein one or more (e.g., 1, 2, 3, or 4) of the carbon atoms in the chain is optionally replaced with a non-peroxide —O—, —S— or —NH— and wherein one or more (e.g., 1, 2, 3, or 4) of the carbon atoms in the chain is replaced with an aryl or heteroaryl ring.

In another embodiment, the linker is a divalent branched or unbranched carbon chain comprising from about 2 to about 30 carbon atoms, which chain optionally includes one or more (e.g., 1, 2, 3, or 4) double or triple bonds, and which chain is optionally substituted with one or more (e.g., 2, 3, or 4) hydroxy or oxo (═O) groups, wherein one or more (e.g., 1, 2, 3, or 4) of the carbon atoms in the chain is replaced with a non-peroxide —O—, —S— or —NH— and wherein one or more (e.g., 1, 2, 3, or 4) of the carbon atoms in the chain is replaced with one or more (e.g., 1, 2, 3, or 4) aryl or heteroaryl rings.

In another embodiment, the linker is a divalent branched or unbranched carbon chain comprising from about 2 to about 30 carbon atoms, which chain optionally includes one or more (e.g., 1, 2, 3, or 4) double or triple bonds, and which chain is optionally substituted with one or more (e.g., 2, 3, or 4) hydroxy or oxo (═O) groups, wherein one or more (e.g., 1, 2, 3, or 4) of the carbon atoms in the chain is replaced with a non-peroxide —O—, —S— or —NH— and wherein one or more (e.g., 1, 2, 3, or 4) of the carbon atoms in the chain is replaced with one or more (e.g., 1, 2, 3, or 4) heteroaryl rings.

In another embodiment, the linker is a divalent branched or unbranched carbon chain comprising from about 2 to about 30 carbon atoms, which chain optionally includes one or more (e.g., 1, 2, 3, or 4) double or triple bonds, and which chain is optionally substituted with one or more (e.g., 2, 3, or 4) hydroxy or oxo (═O) groups, wherein one or more (e.g., 1, 2, 3, or 4) of the carbon atoms in the chain is optionally replaced with a non-peroxide —O—, —S— or —NH—.

In another embodiment, the linker is a divalent group of the formula —W—F—W— wherein F is (C₁-C₃₀)alkyl, (C₂-C₃₀)alkenyl, (C₂-C₃₀)alkynyl, (C₃-C₈)cycloalkyl, or (C₆-C₁₀), wherein W is —N(O)C(═O)—, —C(═O)N(O)—, —OC(═O)—, —C(═O)O—, —O—, —S—, —S(O)—, —S(O)₂—, —N(O)—, —C(═O)—, or a direct bond; wherein each Q is independently H or (C₁-C₆)alkyl

In another embodiment, the linker is a divalent branched or unbranched carbon chain comprising from about 2 to about 30 carbon atoms, which chain optionally includes one or more (e.g., 1, 2, 3, or 4) double or triple bonds, and which chain is optionally substituted with one or more (e.g., 2, 3, or 4) hydroxy or oxo (═O) groups.

In another embodiment, the linker is a divalent branched or unbranched carbon chain comprising from about 2 to about 30 carbon atoms, which chain optionally includes one or more (e.g., 1, 2, 3, or 4) double or triple bonds.

In another embodiment, the linker is a divalent branched or unbranched carbon chain comprising from about 2 to about 30 carbon atoms.

In another embodiment, the linker is a divalent branched or unbranched carbon chain comprising from about 2 to about 20 carbon atoms, which chain optionally includes one or more (e.g., 1, 2, 3, or 4) double or triple bonds, and which chain is optionally substituted with one or more (e.g., 2, 3, or 4) hydroxy or oxo (═O) groups.

In another embodiment, the linker is a divalent branched or unbranched carbon chain comprising from about 2 to about 20 carbon atoms, which chain optionally includes one or more (e.g., 1, 2, 3, or 4) double or triple bonds.

In another embodiment, the linker is a divalent branched or unbranched carbon chain comprising from about 2 to about 20 carbon atoms.

In another embodiment, the linker is —(CH₂CH₂O)—₁₋₁₀.

In another embodiment, the linker is —C(═O)NH(CH₂)₃—; —C(═O)NH(CH₂)₅C(═O)NH(CH₂)—; —CH₂OC(═O)NH(CH₂)₂O(CH₂)₂O(CH₂)—; —C(═O)NH(CH₂)₂O(CH₂)₂O(CH₂)₃—; —CH₂OC(═O)NH(CH₂)₂O(CH₂)₂O(CH₂)₃—; —(CH₂)₄C(═O)NH(CH₂)₂O(CH₂)₂O(CH₂)₃—; —C(═O)NH(CH₂)₅C(═O)NH(CH₂)₂O(CH₂)₂O(CH₂)₃—.

In another embodiment, the linker comprises one or more divalent heteroaryl groups.

Specifically, (C₁-C₃₀)alkyl can be methyl, ethyl, propyl, isopropyl, butyl, iso-butyl, sec-butyl, pentyl, 3-pentyl, hexyl, heptyl, octyl, nonyl, or decyl; (C₃-C₈)cycloalkyl can be cyclopropyl, cyclobutyl, cyclopentyl, or cyclohexyl; (C₂-C₃₀)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, 5-hexenyl, heptenyl, octenyl, nonenyl, or decenyl; (C₂-C₃₀)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, heptynyl, octynyl, nonynyl, or decynyl; (C₆-C₁₀)aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The term aromatic includes aryl and heteroaryl groups.

Aryl denotes a phenyl radical or an ortho-fused bicyclic carbocyclic radical having about nine to ten ring atoms in which at least one ring is aromatic.

Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

The term “amino acid,” when used with reference to a linker, comprises the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also includes natural and unnatural amino acids bearing a conventional amino protecting group (e.g., acetyl or benzyloxycarbonyl), as well as natural and unnatural amino acids protected at the carboxy terminus (e.g. as a (C₁-C₆)alkyl, phenyl or benzyl ester or amide). Other suitable amino and carboxy protecting groups are known to those skilled in the art (see for example, Greene, Protecting Groups In Organic Synthesis; Wiley: New York, 1981, and references cited therein). An amino acid can be linked to another molecule through the carboxy terminus, the amino terminus, or through any other convenient point of attachment, such as, for example, through the sulfur of cysteine.

The term “peptide” when used with reference to a linker, describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to another molecule through the carboxy terminus, the amino terminus, or through any other convenient point of attachment, such as, for example, through the sulfur of a cysteine. Preferably a peptide comprises 3 to 25, or 5 to 21 amino acids. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right.

### Exemplary Substrates

In one embodiment, the hydrolase substrate has a compound of formula (I): R-linker-A-X, wherein R is one or more functional groups, wherein the linker is a multiatom straight or branched chain including C, N, S, or O, or a group that comprises one or more rings, e.g., saturated or unsaturated rings, such as one or more aryl rings, heteroaryl rings, or any combination thereof, wherein A-X is a substrate for a dehalogenase, e.g., a haloalkane dehalogenase or a dehalogenase that cleaves carbon-halogen bonds in an aliphatic or aromatic halogenated substrate, such as a substrate for Rhodococcus, Sphingomonas, Staphylococcus, Pseudomonas, Burkholderia, Agrobacterium or Xanthobacter dehalogenase, and wherein X is a halogen. In one embodiment, an alkylhalide is covalently attached to a linker, L, which is a group or groups that covalently attach one or more functional groups to form a substrate for a dehalogenase.

In one embodiment, a substrate of the invention for a dehalogenase which has a linker has the formula (I):

R-linker-A-X (I)

wherein R is one or more functional groups (such as a fluorophore, biotin, luminophore, or a fluorogenic or luminogenic molecule, or is a solid support, including microspheres, membranes, polymeric plates, glass beads, glass slides, and the like), wherein the linker is a multiatom straight or branched chain including C, N, S, or O, wherein A-X is a substrate for a dehalogenase, and wherein X is a halogen. In one embodiment, A-X is a haloaliphatic or haloaromatic substrate for a dehalogenase. In one embodiment, the linker is a divalent branched or unbranched carbon chain comprising from about 12 to about 30 carbon atoms, which chain optionally includes one or more (e.g., 1, 2, 3, or 4) double or triple bonds, and which chain is optionally substituted with one or more (e.g., 2, 3, or 4) hydroxy or oxo (═O) groups, wherein one or more (e.g., 1, 2, 3, or 4) of the carbon atoms in the chain is optionally replaced with a non-peroxide —O—, —S— or —NH—. In one embodiment, the linker comprises 3 to 30 atoms, e.g., 11 to 30 atoms. In one embodiment, the linker comprises (CH₂CH₂O)_{y} and y=2 to 8. In one embodiment, A is (CH₂)ₙ and n=2 to 10, e.g., 4 to 10. In one embodiment, A is CH₂CH₂ or CH₂CH₂CH₂. In another embodiment, A comprises an aryl or heteroaryl group. In one embodiment, a linker in a substrate for a dehalogenase such as a Rhodococcus dehalogenase, is a multiatom straight or branched chain including C, N, S, or O, and preferably 11-30 atoms when the functional group R includes an aromatic ring system or is a solid support.

In another embodiment, a substrate of the invention for a dehalogenase which has a linker has formula (II):

R-linker-CH₂—CH₂—CH₂—X (II)

where X is a halogen, preferably chloride. In one embodiment, R is one or more functional groups, such as a fluorophore, biotin, luminophore, or a fluorogenic or luminogenic molecule, or is a solid support, including microspheres, membranes, glass beads, and the like. When R is a radiolabel, or a small detectable atom such as a spectroscopically active isotope, the linker can be 0-30 atoms.

Exemplary dehalogenase substrates are described in U.S. published application numbers 2006/0024808 and 2005/0272114, which are incorporated by reference herein.

### Exemplary Mutant Dehalogenases for Use in Split Hydrolases

Carboxytetramethylrhodamine-C₁₀H₂₁NO₂—Cl, carboxyfluorescein-C₁₀H₂₁NO₂—Cl, and 5-carboxy-X-rhodamine-C₁₀H₂₁NO₂—Cl bound to DhaA.H272F but not to DhaA.WT. Biotin-C₁₀H₂₁NO₂—Cl bound to DhaA.H272F but not to DhaA.WT. The bond between substrates and DhaA.H272F was very strong, since boiling with SDS did not break the bond.

DhaA.H272 mutants, i.e. H272F/G/A/Q, bound to carboxytetramethylrhodamine-C₁₀H₂₁NO₂—Cl. The DhaA.H272 mutants bind the substrates in a highly specific manner, since pretreatment of the mutants with one of the substrates (biotin-C₁₀H₂₁NO₂—Cl) completely blocked the binding of another substrate (carboxytetramethylrhodamine-C₁₀H₂₁NO₂—Cl).

D at residue 106 in DhaA was substituted with nucleophilic amino acid residues other than D, e.g., C, Y and E, which may form a bond with a substrate which is more stable than the bond formed between wild-type DhaA and the substrate. In particular, cysteine is a known nucleophile in cysteine-based enzymes, and those enzymes are not known to activate water.

A control mutant, DhaA.D106Q, single mutants DhaA.D106C, DhaA.D106Y, and DhaA.D106E, as well as double mutants DhaA.D106C:H272F, DhaA.D106E:H272F, DhaA.D106Q:H272F, and DhaA.D106Y:H272F were analyzed for binding to carboxytetramethylrhodamine-C₁₀H₂₁NO₂—Cl. Carboxytetramethylrhodamine-C₁₀H₂₁NO₂—Cl bound to DhaA.D106C, DhaA.D106C:H272F, DhaA.D106E, and DhaA.H272F. Thus, the bond formed between carboxytetramethylrhodamine-C₁₀H₂₁NO₂—Cl and cysteine or glutamate at residue 106 in a mutant DhaA is stable relative to the bond formed between carboxytetramethylrhodamine-C₁₀H₂₁NO₂—Cl and DhaA.WT. Other substitutions at position 106 alone or in combination with substitutions at other residues in DhaA may yield similar results. Further, certain substitutions at position 106 alone or in combination with substitutions at other residues in DhaA may result in a mutant DhaA that forms a bond with only certain substrates.

In one embodiment, the mutant dehalogenase of the invention comprises at least two amino acid substitutions, at least one of which is associated with stable bond formation, e.g., a residue in the wild-type hydrolase that activates the water molecule, e.g., a histidine residue, and is at a position corresponding to amino acid residue 272 of a Rhodococcus rhodochrous dehalogenase, e.g., the substituted amino acid is asparagine, glycine or phenylalanine, and at least one other is associated with improved functional expression, binding kinetics or FP signal, e.g., at a position corresponding to position 5, 11, 20, 30, 32, 47, 58, 60, 65, 78, 80, 87, 88, 94, 109, 113, 117, 118, 124, 128, 134, 136, 150, 151, 155, 157, 160, 167, 172, 187, 195, 204, 221, 224, 227, 231, 250, 256, 257, 263, 264, 277, 282, 291 or 292 of SEQ ID NO:1.

### Identification of Residues for Mutagenesis

Residue numbering is based on the primary sequence of DhaA, which differs from numbering in the published crystal structure (1BN6.pdb). Using the DhaA substrate model, dehalogenase residues within 3 Å and 5 Å of the bound substrate were identified. These residues represented the first potential targets for mutagenesis. From this list residues were selected, which, when replaced, would likely remove steric hindrances or unfavorable interactions, or introduce favorable charge, polar, or other interactions. For instance, the Lys residue at position 175 is located on the surface of DhaA at the substrate tunnel entrance: removal of this large charged side chain might improve substrate entry into the tunnel. The Cys residue at position 176 lines the substrate tunnel and its bulky side chain causes a constriction in the tunnel: removal of this side chain might open up the tunnel and improve substrate entry. The Val residue at position 245 lines the substrate tunnel and is in close proximity to two oxygens of the bound substrate: replacement of this residue with threonine may add hydrogen bonding opportunities that might improve substrate binding. Lastly, Bosma et al. (2002) reported the isolation of a catalytically proficient mutant of DhaA with the amino acid substitution Tyr273Phe. This mutation, when recombined with a Cys176Tyr substitution, resulted in an enzyme that was nearly eight times more efficient in dehalogenating 1,2,3-trichloropropane (TCP) than the wild type dehalogenase. Based on these structural analyses, the codons at positions 175, 176 and 273 were randomized, in addition to generating the site-directed V245T mutation. The resulting mutants were screened for improved rates of covalent bond formation with fluorescent (e.g., a compound of formula VI or VIII) and biotin coupled DhaA substrates.

### Library Generation and Screening

The starting material for all library and mutant constructions were pGEX5X3 based plasmids containing genes encoding DhaA.H272F and DhaA.D106C. These plasmids harbor genes that encode the parental DhaA mutants capable of forming stable covalent bonds with haloalkane ligands. Codons at positions 175, 176 and 273 in the DhaA.H272F and DhaA.D106C templates were randomized using a NNK site-saturation mutagenesis strategy. In addition to the single-site libraries at these positions, combination 175/176 NNK libraries were also constructed.

Three assays were evaluated as the primary screening tool for the DhaA mutant libraries. The first, an in vivo labeling assay, was based on the assumption that improved DhaA mutants in E. coli would have superior labeling properties. Following a brief labeling period with carboxytetramethylrhodamine-C₁₀H₂₁NO₂—Cl and cell wash, superior clones should have higher levels of fluorescent intensity at 575 nm. Screening of just one 96 well plate of the DhaA.H272F 175/176 library was successful in identifying several potential improvements (i.e., hits). Four clones had intensity levels that were 2-fold higher than the parental clone. Despite the potential usefulness of this assay, however, it was not chosen as the primary screen because of the difficulties encountered with automation procedures and due to the fact that simple overexpression of active DhaA mutants could give rise to false positives.

The second assay that was considered as a primary screen was an in vitro assay that effectively normalized for protein concentration by capturing saturating amounts of DhaA mutants on immobilized anti-FLAG antibody in a 96 well format. Like the in vivo assay, this assay was also able to clearly identify potential improved DhaA mutants from a large background of parental activities. Several clones produced signals up to 4-fold higher than the parent DhaA.H272F. This assay, however, was costly due to reagent expense and assay preparation time, and the automation of multiple incubation and washing steps. In addition, this assay was unable to capture some mutants that were previously isolated and characterized as being superior.

An automated MagneGST™-based assay was used to screen the DhaA mutant protein libraries. Screening of the DhaA.H272F and DhaA.D106C-based 175 single-site libraries failed to reveal hits that were significantly better than the parental clones. The screen identified several clones with superior labeling properties compared to the parental controls. Three clones with significantly higher labeling properties could be clearly distinguished from the background which included the DhaA.H272F parent. For clones with at least 50% higher activity than the DhaA.H272F parent, the overall hit rate of the libraries examined varied from between 1-3%. Similar screening results were obtained for the DhaA.D106C libraries (data not shown). The hits identified by the initial primary screen were located in the master plates, consolidated, re-grown and reanalyzed using the MagneGST™ assay. Only those DhaA mutants with at least a 2-fold higher signal than the parental control upon reanalysis were chosen for sequence analysis.

### Sequence Analysis of DhaA Hits

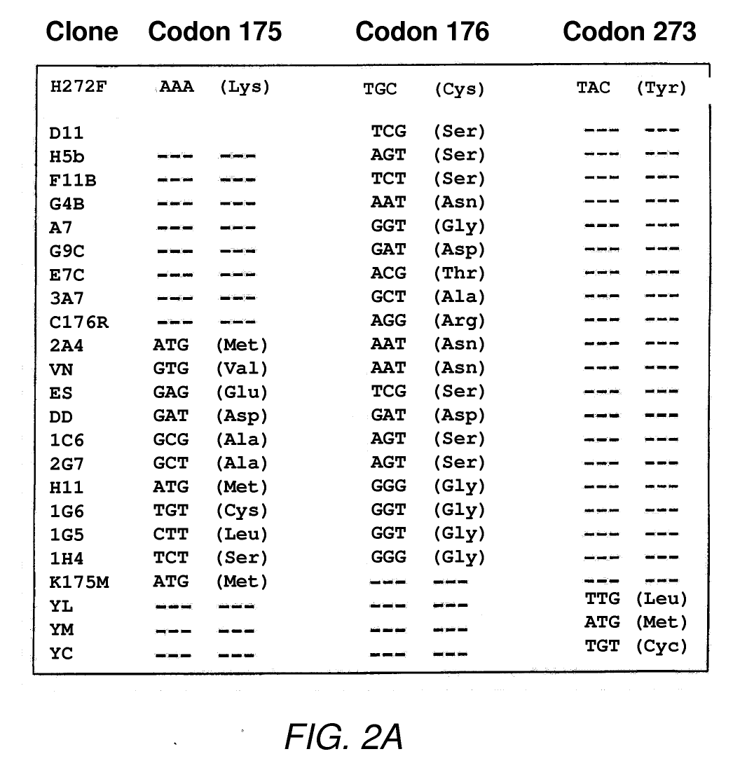

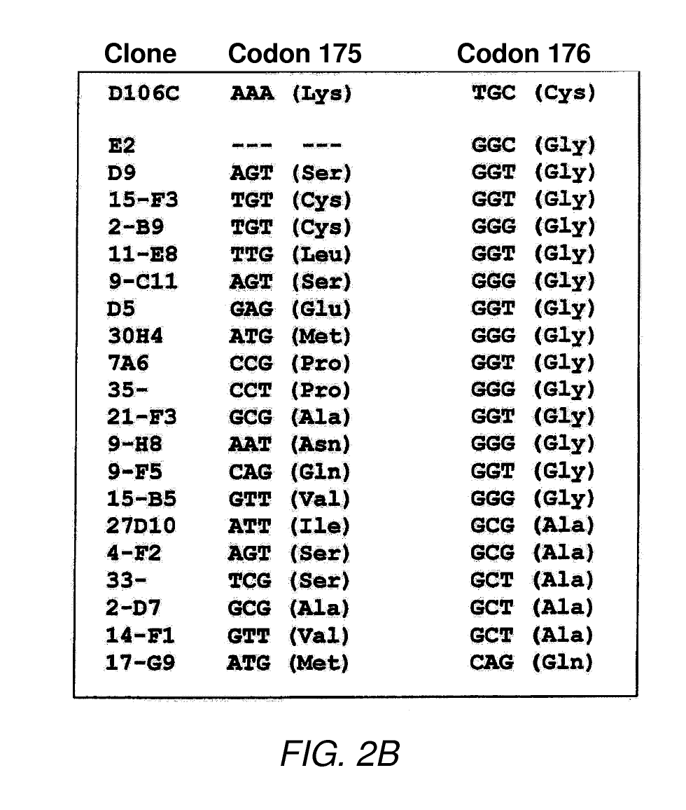

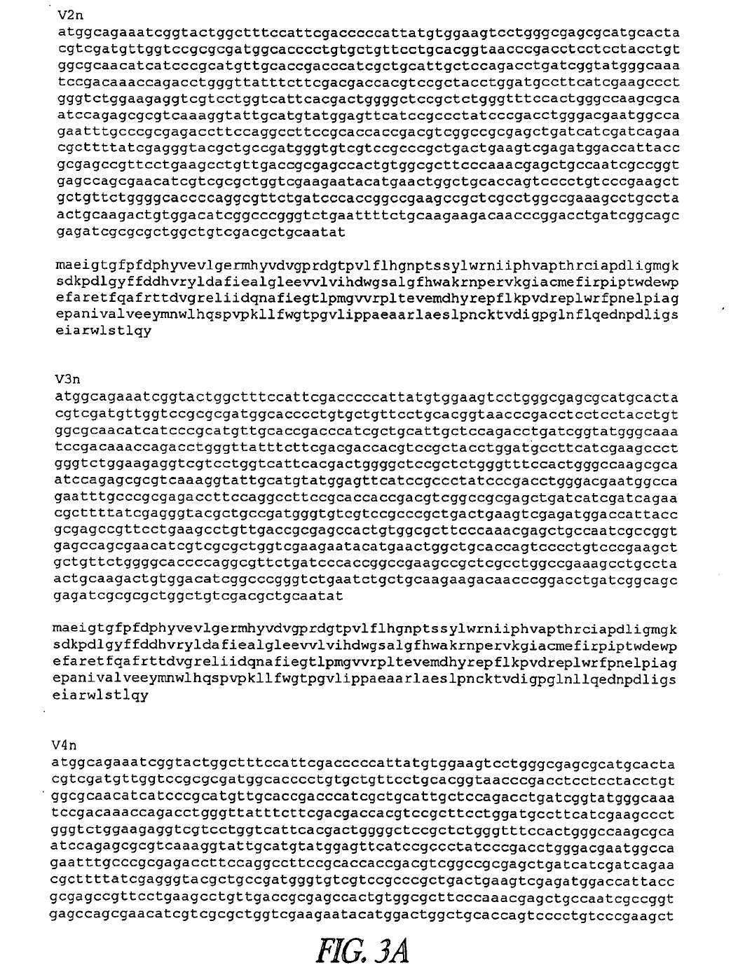

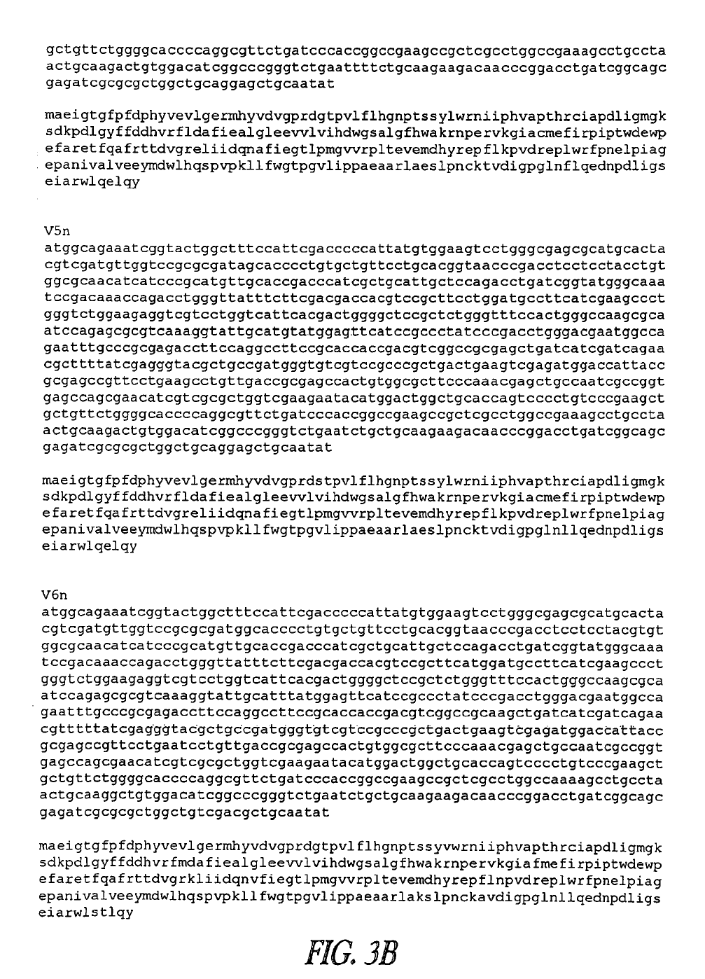

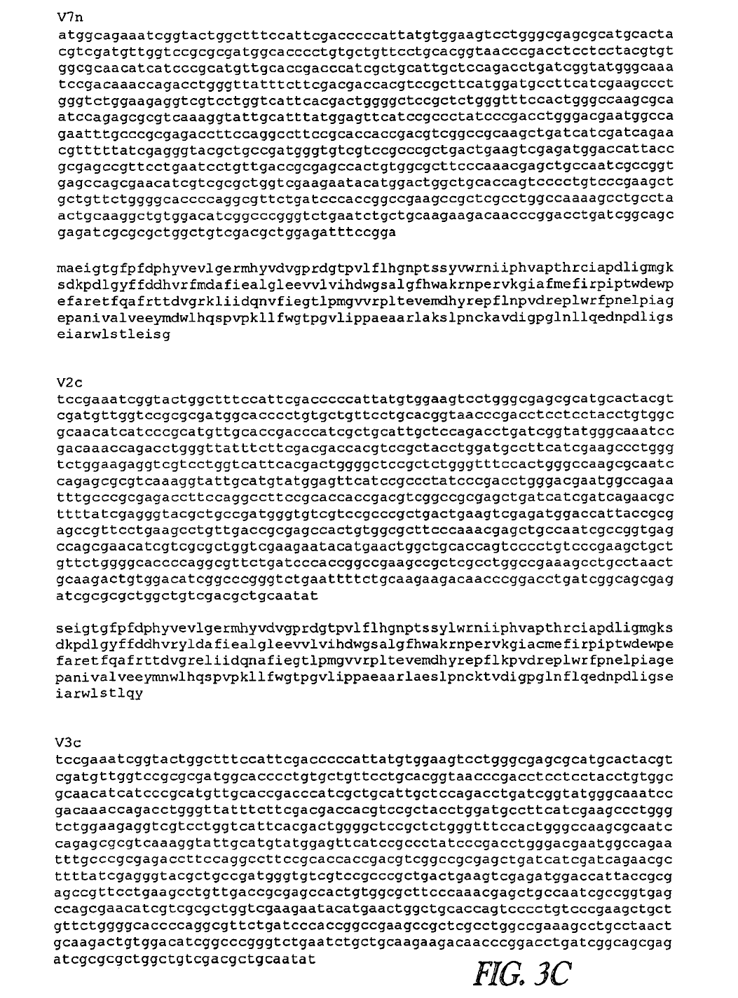

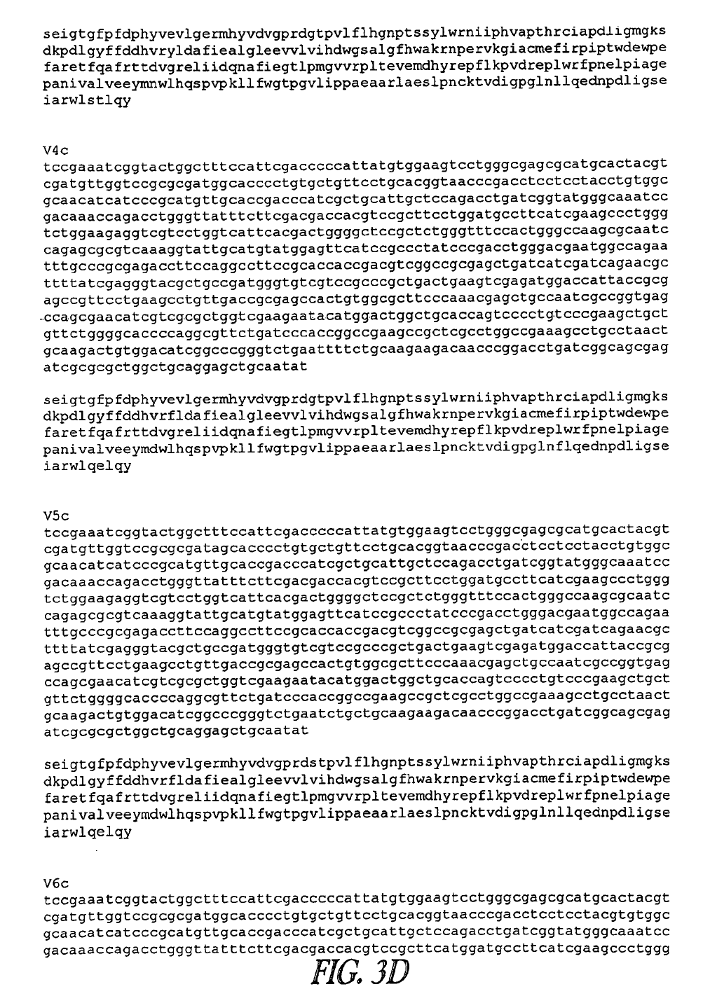

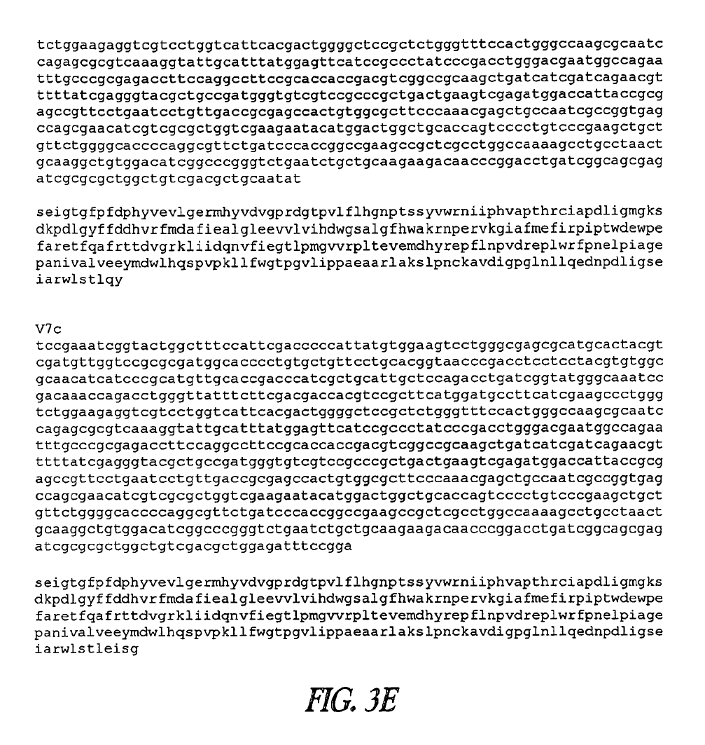

FIG. 2A shows the codons of the DhaA mutants identified following screening of the DhaA.H272F libraries. This analysis identified seven single 176 amino acid substitutions (C176G, C176N, C176S, C176D, C176T and C176A, and C176R). Interestingly, three different serine codons were isolated. Numerous double amino acid substitutions at positions 175 and 176 were also identified (K175E/C176S, K175C/C176G, K175M/C176G, K175L/C176G, K175S/C176G, K175V/C176N, K175A/C176S, and K175M/C176N). While seven different amino acids were found at the 175 position in these double mutants, only three different amino acids (Ser, Gly and Asn) were identified at position 176. A single K175M mutation identified during library quality assessment was included in the analysis. In addition, several superior single Y273 substitutions (Y273C, Y273M, Y273L) were also identified.

FIG. 2B shows the mutated codons of the DhaA mutants identified in the DhaA.D106C libraries. Except for the single C176G mutation, most of the clones identified contained double 175/176 mutations. A total of 11 different amino acids were identified at the 175 position. In contrast, only three amino acids (Gly, Ala and Gln) were identified at position 176 with Gly appearing in almost ¾ of the D106C double mutants.

### Characterization of DhaA Mutants

Several DhaA.H272F and D106C-based mutants identified by the screening procedure produced significantly higher signals in the MagneGST assay than the parental clones. DhaA.H272F based mutants A7 and H11, as well as the DhaA.D106C based mutant D9, generated a considerably higher signal with carboxytetramethylrhodamine-C₁₀H₂₁NO₂—Cl than the respective parents. In addition, all of the DhaA.H272F based mutants identified at the 273 position (Y273L “YL”, Y273M “YM”, and Y273C “YC”) appeared to be significantly improved over the parental clones using the biotin-PEG4-14-Cl substrate. The results of these analyses were consistent with protein labeling studies using SDS-PAGE fluorimage gel analysis. In an effort to determine if combinations of the best mutations identified in the DhaA.H272F background were additive, the three mutations at residue 273 were recombined with the DhaA.H272F A7 and DhaA.H272F H11 mutations. In order to distinguish these recombined protein mutants from the mutants identified in round one of screening (first generation), they are referred to as “second generation” DhaA mutants.

To facilitate comparative kinetic studies several improved DhaA mutants were selected for purification using a Glutathione Sepharose 4B resin. In general, production of DhaA.H272F and DhaA.D106C based fusions in E. coli was robust, although single amino acid changes may have negative consequences on the production of DhaA. As a result of this variability in protein production, the overall yield of the DhaA mutants also varied considerably (1-15 mg/mL). Preliminary kinetic labeling studies were performed using several DhaA.H272F derived mutants. Many, if not all, of the mutants chosen for analysis had faster labeling kinetics than the H272F parent. In fact, upon closer inspection of the time course, the labeling of several DhaA mutants including the first generation mutant YL and the two second generation mutants, A7YM and H11YL mutants appeared to be complete by 2 minutes. A more expanded time course analysis was performed on the DhaA.H272F A7 and the two second generation DhaA.H272F mutants A7YM and H11YL. The labeling reactions of the two second generation clones are for the most part complete by the first time point (20 seconds). The A7 mutant, on the other hand, appears only to be reaching completion by the last time point (7 minutes). The fluorescent bands on gel were quantitated and the relative rates of product formation determined. In order to determine a labeling rate, the concentration of the H11YL was reduced from 50 ng to 10 ng and a more refined time-course was performed. Under these labeling conditions a linear initial rate could be measured. Quantitation of the fluorimaged gel data allowed second order rate constants to be calculated. Based on the slope observed, the second order rate constant for carboxytetramethylrhodamine-C₁₀H₂₁NO₂—Cl labeling of DhaA.H272F H11YL was 5.0×10⁵ M⁻¹ sec⁻¹.

Fluorescence polarization (FP) is ideal for the study of small fluorescent ligands binding to proteins. It is unique among methods used to analyze molecular binding because it gives direct nearly instantaneous measure of a substrate bound/free ratio. Therefore, an FP assay was developed as an alternative approach to fluorimage gel analysis of the purified DhaA mutants. Under the labeling conditions used, the second generation mutant DhaA.H272F H11YL was significantly faster than its A7 and H272F counterparts. To place this rate in perspective, approximately 42 and 420-fold more A7 and parental, i.e., DhaA.H272F, protein, respectively, was required in the reaction to obtain measurable rates. Under the labeling conditions used, it is evident that the H11YL mutant was also considerably faster than A7 and parental, DhaA.H272F proteins with the fluorescein-based substrate. However, it appears that labeling of H11YL with carboxyfluorescein-C₁₀H₂₁NO₂—Cl is markedly slower than labeling with the corresponding carboxytetramethylrhodamine-C₁₀H₂₁NO₂—Cl substrate. Four-fold more H11YL protein was used in the carboxyfluorescein-C₁₀H₂₁NO₂—Cl reaction (150 nM) versus the carboxytetramethylrhodamine-C₁₀H₂₁NO₂—Cl reaction (35 nM), yet the rate observed appeared to be qualitatively slower than the observed carboxytetramethylrhodamine-C₁₀H₂₁NO₂—Cl rate.

Based on the sensitivity and truly homogenous nature of this assay, FP was used to characterize the labeling properties of the purified DhaA mutants with the fluorescently coupled substrates. The data from these studies was then used to calculate a second order rate constant for each DhaA mutant-substrate pair. The two parental proteins used in this study, DhaA.H272F and DhaA.D106C, were found to have comparable rates with the carboxytetramethylrhodamine and carboxyfluorescein-based substrates. However, in each case labeling was slower with the carboxyfluorescein-C₁₀H₂₁NO₂—Cl substrate. All of the first generation DhaA mutants characterized by FP had rates that ranged from 7 to 3555-fold faster than the corresponding parental protein. By far, the biggest impact on labeling rate by a single amino acid substitution occurred with the three replacements at the 273 position (Y273L, Y273M, and Y273C) in the DhaA.H272F background. Nevertheless, in each of the first generation DhaA.H272F mutants tested, labeling with the carboxyfluorescein-C₁₀H₂₁NO₂—Cl substrate always occurred at a slower rate (1.6 to 46-fold). Most of the second generation DhaA.H272F mutants were significantly faster than even the most improved first generation mutants. One mutant in particular, H11YL, had a calculated second order rate constant with carboxytetramethylrhodamine-C₁₀H₂₁NO₂—Cl that was over four orders of magnitude higher than the DhaA.H272F parent. The H11YL rate constant of 2.2×10⁶ M⁻¹ sec⁻¹ was nearly identical to the rate constant calculated for a carboxytetramethylrhodamine-coupled biotin/streptavidin interaction. This value is consistent with an on-rate of 5×10⁶ M⁻¹ sec¹ determined for a biotin-streptavidin interaction using surface plasmon resonance analysis (Qureshi et al., 2001). Several of the second generation mutants also had improved rates with the carboxyfluorescein-C₁₀H₂₁NO₂—Cl substrate, however, as noted previously, these rates were always slower than with the carboxytetramethylrhodamine-C₁₀H₂₁NO₂—Cl substrate. For example, the carboxyfluorescein-C₁₀H₂₁NO₂—Cl labeling rate of the DhaA.H272F H11YL mutant was 100-fold lower than the carboxytetramethylrhodamine-C₁₀H₂₁NO₂—Cl labeling rate.

### Exemplary Methods

The invention provides methods to monitor the expression, location and/or trafficking of molecules in a cell, as well as to monitor changes in microenvironments within a cell, e.g., to image, identify, localize, display or detect one or more molecules which may be present in a sample, e.g., in a cell, which methods employ a hydrolase substrate and a split mutant hydrolase system. The hydrolase substrates employed in the methods of the invention are preferably soluble in an aqueous or mostly aqueous solution, including water and aqueous solutions having a pH greater than or equal to about 6. Stock solutions of substrates, however, may be dissolved in organic solvent before diluting into aqueous solution or buffer. Preferred organic solvents are aprotic polar solvents such as DMSO, DMF, N-methylpyrrolidone, acetone, acetonitrile, dioxane, tetrahydrofuran and other nonhydroxylic, completely water-miscible solvents. The concentration of a hydrolase substrate and a split mutant hydrolase to be used is dependent upon the experimental conditions and the desired results, e.g., to obtain results within a reasonable time, with minimal background or undesirable labeling. The concentration of a hydrolase substrate typically ranges from nanomolar to micromolar. The required concentration for the hydrolase substrate with a corresponding split mutant hydrolase is determined by systematic variation in substrate until satisfactory labeling is accomplished. The starting ranges are readily determined from methods known in the art.

In one embodiment, a substrate which includes a functional group with optical properties is employed to detect an interaction between a cellular molecule and a fusion partner of a fusion having a hydrolase fragment. Such a substrate is combined with the sample of interest comprising the fusion and a second hydrolase fragment for a period of time sufficient for the fusion partner to bind the cellular molecule, e.g., after activation of the molecule, and the two hydrolase fragments to associate and to bind the substrate, after which the sample is illuminated at a wavelength selected to elicit the optical response of the functional group. Optionally, the sample is washed to remove residual, excess or unbound substrate. In one embodiment, the labeling is used to determine a specified characteristic of the sample by further comparing the optical response with a standard or expected response. For example, the mutant hydrolase bound substrate is used to monitor specific components of the sample with respect to their spatial and temporal distribution in the sample. Alternatively, the mutant hydrolase bound substrate is employed to determine or detect the presence or quantity of a certain molecule.

In contrast to intrinsically fluorescent proteins, e.g., GFP, a fragment of a mutant hydrolase bound to a fluorescent substrate does not require a native protein structure to retain fluorescence. After the fluorescent substrate is bound, the fragment of a mutant hydrolase may be detected, for example, in denaturing electrophoretic gels, e.g., SDS-PAGE, or in cells fixed with organic solvents, e.g., paraformaldehyde.

A detectable optical response means a change in, or occurrence of, a parameter in a test system that is capable of being perceived, either by direct observation or instrumentally. Such detectable responses include the change in, or appearance of, color, fluorescence, reflectance, chemiluminescence, light polarization, light scattering, or X-ray scattering. Typically the detectable response is a change in fluorescence, such as a change in the intensity, excitation or emission wavelength distribution of fluorescence, fluorescence lifetime, fluorescence polarization, or a combination thereof. The detectable optical response may occur throughout the sample or in a localized portion of the sample having the substrate bound to the hydrolase fragment. Comparison of the degree of optical response with a standard or expected response can be used to determine whether and to what degree the sample possesses a given characteristic.

A sample comprising a split hydrolase is typically labeled by passive means, i.e., by incubation with the substrate. However, any method of introducing the substrate into the sample such as microinjection of a substrate into a cell or organelle, can be used to introduce the substrate into the sample. The substrates of the present invention are generally non-toxic to living cells and other biological components, within the concentrations of use.

A sample comprising a split mutant hydrolase can be observed immediately after contact with a substrate of the invention. The sample comprising a split mutant hydrolase or a fusion thereof is optionally combined with other solutions in the course of labeling, including wash solutions, permeabilization and/or fixation solutions, and other solutions containing additional detection reagents. Washing following contact with the substrate may improve the detection of the optical response due to the decrease in non-specific background after washing. Satisfactory visualization is possible without washing by using lower labeling concentrations. A number of fixatives and fixation conditions are known in the art, including formaldehyde, paraformaldehyde, formalin, glutaraldehyde, cold methanol and 3:1 methanol:acetic acid. Fixation is typically used to preserve cellular morphology and to reduce biohazards when working with pathogenic samples. Selected embodiments of the substrates are well retained in cells. Fixation is optionally followed or accompanied by permeabilization, such as with acetone, ethanol, DMSO or various detergents, to allow bulky substrates of the invention, to cross cell membranes, according to methods generally known in the art. Optionally, the use of a substrate may be combined with the use of an additional detection reagent that produces a detectable response due to the presence of a specific cell component, intracellular substance, or cellular condition, in a sample comprising a mutant hydrolase or a fusion thereof. Where the additional detection reagent has spectral properties that differ from those of the substrate, multi-color applications are possible.

At any time after or during contact with the substrate having a functional group with optical properties, the sample comprising a hydrolase fragment or a fusion thereof is illuminated with a wavelength of light that results in a detectable optical response, and observed with a means for detecting the optical response. While some substrates are detectable colorimetrically, using ambient light, other substrates are detected by the fluorescence properties of the parent fluorophore. Upon illumination, such as by an ultraviolet or visible wavelength emission lamp, an arc lamp, a laser, or even sunlight or ordinary room light, the substrates, including substrates bound to the complementary specific binding pair member, display intense visible absorption as well as fluorescence emission. Selected equipment that is useful for illuminating the substrates of the invention includes, but is not limited to, hand-held ultraviolet lamps, mercury arc lamps, xenon lamps, argon lasers, laser diodes, and YAG lasers. These illumination sources are optionally integrated into laser scanners, fluorescence microplate readers, standard or mini fluorometers, or chromatographic detectors. This colorimetric absorbance or fluorescence emission is optionally detected by visual inspection, or by use of any of the following devices: CCD cameras, video cameras, photographic film, laser scanning devices, fluorometers, photodiodes, quantum counters, epifluorescence microscopes, scanning microscopes, flow cytometers, fluorescence microplate readers, or by means for amplifying the signal such as photomultiplier tubes. Where the sample comprising a mutant hydrolase or a fusion thereof is examined using a flow cytometer, a fluorescence microscope or a fluorometer, the instrument is optionally used to distinguish and discriminate between the substrate comprising a functional group which is a fluorophore and a second fluorophore with detectably different optical properties, typically by distinguishing the fluorescence response of the substrate from that of the second fluorophore. Where the sample is examined using a flow cytometer, examination of the sample optionally includes isolation of particles within the sample based on the fluorescence response of the substrate by using a sorting device.

The invention will be described by the following non-limiting examples.

### Example 1

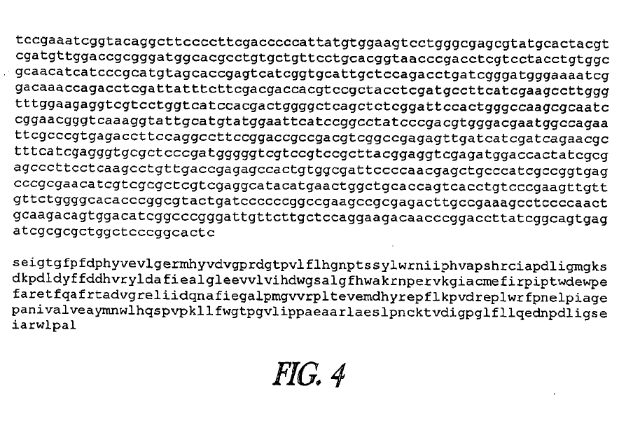

The following site-directed changes to DNA for DhaA.H272F H11YL (FIG. 4; HT2, SEQ ID NO:17) were made and found to improve functional expression in E. coli: D78G, F80S, P291A, and P291G, relative to DhaA.H272F H11YL.

Site-saturation mutagenesis at codons 80, 272, and 273 in DhaA.H272F H11YL was employed to create libraries containing all possible amino acids at each of these positions. The libraries were overexpressed in E. coli and screened for functional expression/improved kinetics using a carboxyfluoroscein (FAM) containing dehalogenase substrate (C₃₁H₃₁ClNO₈) and fluorescence polarization (FP). The nature of the screen allowed the identification of protein with improved expression as well as improved kinetics. In particular, the screen excluded mutants with slower intrinsic kinetics. Substitutions with desirable properties included the following: F80Q, F80N, F80K, F80H, F80T, H272N, H272Y, Y273F, Y273M, and Y273L. Of these, Y273F showed improved intrinsic kinetics.

The Phe at 272 in HT2 lacks the ability to hydrogen bond with Glu-130. The interaction between His-272 and Glu-130 is thought to play a structural role, and so the absence of this bond may destabilize HT2. Moreover, the proximity of the Phe to the Tyr->Leu change at position 273 may provide for potentially cooperative interactions between side chains from these adjacent residues. Asn was identified as a better residue for position 272 in the context of either Leu or Phe at position 273. When the structure of HT2 containing Asn-272 was modeled, it was evident that 1) Asn fills space with similar geometry compared to His, and 2) Asn can hydrogen bond with Glu-130. HT2 with a substitution of Asn at position 272 was found to produce higher levels of functional protein in E. coli, cell-free systems, and mammalian cells, likely as a result of improving the overall stability of the protein.

Two rounds of mutagenic PCR were used to introduce mutations across the entire coding sequence for HT2 at a frequency of 1-2 amino acid substitutions per sequence. This approach allowed targeting of the whole sequence and did not rely on any a priori knowledge of HT2 structure/function. In the first round of mutagenesis, Asn-272, Phe-273, and Gly-78 were fixed in the context of an N-terminal HT2 fusion to a humanized Renilla luciferase as a template. Six mutations were identified that were beneficial to improved FP signal for the FAM ligand (S58T, A155T, A172T, A224E, P291S, A292T; V2), and it was determined that each substitution, with the exception of A172T provided increased protein production in E. coli. However, the A172T change provided improved intrinsic kinetics. The 6 substitutions (including Leu+/−273) were then combined to give a composite sequence (V3/V2) that provided significantly improved protein production and intrinsic labeling kinetics when fused to multiple partners and in both orientations.

In the second round of mutagenesis, 6 different templates were used: V3 or V2 were fused at the C-terminus to humanized Renilla luciferase (R^{L}), firefly luciferase, or Id. Mutagenic PCR was carried out as above, and mutations identified as beneficial to at least 2 of the 3 partners were combined to give V6 (Leu-273). In the second round of mutagenic PCR, protein expression was induced using elevated temperature (30° C.) in an attempt to select for sequences conferring thermostability. Increasing the intrinsic structural stability of mutant DhaA fusions may result in more efficient production of protein.

Random mutations associated with desirable properties included the following: G5C, G5R, D11N, E20K, R30S, G32S, L47V, S58T, R60H, D65Y, Y87F, L88M, A94V, S109A, F113L, K117M, R118H, K124I, C128F, P134H, P136T, Q150H, A151T, A155T, V157I, E160K, A167V, A172T, D187G, K195N, R2045, L221M, A224E, N227E, N227S, N227D, Q231H, A250V, A256D, E257K, K263T, T264A, D277N, I282F, P291S, P291Q, A292T, and A292E.

In addition to the substitutions above, substitutions in a connector sequence between the mutant DhaA and the downstream C-terminal partner, Renilla luciferase, were identified. The parental connector sequence (residues 294-320) is: QYSGGGGSGGGGSGGGGENLYFQAIEL (SEQ ID NO:19). The substitutions identified in the connector which were associated with improved FP signal were Y295N, G298C, G302D, G304D, G308D, G310D, L313P, L313Q, and A317E. Notably, five out of nine were negatively charged.

With the exception of A172T and Y273F (in the context of H272N), all of the above substitutions provided improved functional expression in E. coli as N-terminal fusions. Nevertheless, A172T and Y273F improved intrinsic kinetics for labeling.

Exemplary combined substitutions in mutant DhaAs with generally improved properties were:
DhaA 2.3 (V3): S58T, D78G, A155T, A172T, A224E, F272N, P291S, and A292T.
DhaA 2.4 (V4): S58T, D78G, Y87F, A155T, A172T, A224E, N227D, F272N, Y273F, P291Q, and A292E.
DhaA 2.5 (V5): G32S, S58T, D78G, Y87F, A155T, A172T, A224E, N227D, F272N, P291Q, and A292E.
DhaA 2.6 (V6): L47V, S58T, D78G, Y87F, L88M, C128F, A155T, E160K, A167V, A172T, K195N, A224E, N227D, E257K, T264A, F272N, P291S, and A292T.
   Of the substitutions found in DhaA 2.6, all improved functional expression in E. coli with the exception of A167V, which improved intrinsic kinetics.

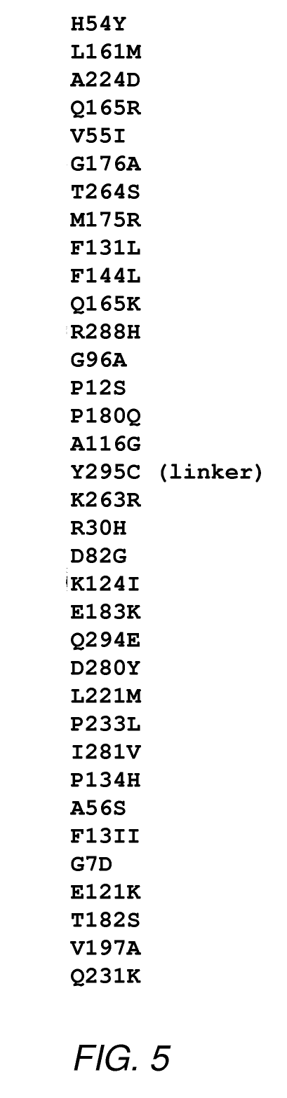

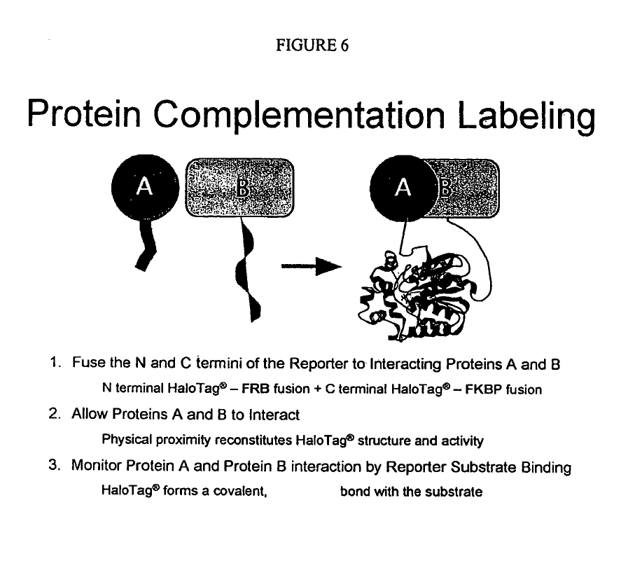

FIG. 5 provides additional substitutions which improve functional expression in E. coli.

The V6 sequence was used as a template for mutagenesis at the C-terminus. A library of mutants was prepared containing random, two-residue extensions (tails) in the context of an Id-V6 fusion (V6 is the C-terminal partner), and screened with the FAM ligand. Mutants with improved protein production and less non-specific cleavage (as determined by TMR ligand labeling and gel analysis) were identified. The two C-terminal residues in DhaA 2.6 (“V6”) were replaced with Glu-Ile-Ser-Gly to yield V7. The expression of V7 was compared to V6 as both an N- and C-terminal fusion to Id. Fusions were overexpressed in E. coli and labeled to completion with 10 μM TMR ligand, then resolved by SDS-PAGE+fluorimaging. The data shows that more functional fusion protein was made from the V7 sequence. In addition, labeling kinetics with a FAM ligand over time for V7 were similar to that for V6, although V7 had faster kinetics than V6 when purified nonfused protein was tested.

To test for in vivo labeling, 24 hours after HeLa cells were transfected with vectors for HT2, V3, V7 and V7F (V7F has a single amino acid difference relative to V7; V7F has Phe at position 273 rather than Leu), cells were labeled in vivo with 0.2 μM TMR ligand for 5 minutes, 15 minutes, 30 minutes or 2 hours. Samples were analyzed by SDS-PAGE/fluorimaging and quantitated by ImageQuant. V7 and V7F resulted in better functional expression than HT2 and V3, and V7, V7F and V3 had improved kinetics in vivo in mammalian cells relative to HT2.

Moreover, V7 has improved functional expression as an N- or C-terminal fusion, and was more efficient in pull down assays than other mutant DhaAs. The results showed that V7>V6>V3 for the quantity of MyoD that can be pulled down using HaloLink™-immobilized mutant DhaA-Id fusions. V7 and V7F had improved labeling kinetics. In particular, V7F had about 1.5- to about 3-fold faster labeling than V7.

Moreover, V7>V6>V7F>V3>HT2 for thermostability. For example, under some conditions (30 minute exposure to 48° C.) purified V7F loses 50% of its activity, while V7 still maintains 80% activity. The thermostability discrepancy between the two is more dramatic when they V7 and V7F are expressed in E. coli and analyzed as lysates.

Note that the ends of these mutants can accommodate various sequences including tail and connector sequences, as well as substitutions. For instance, the N-terminus of a mutant DhaA may be M/GA/SETG (SEQ ID NO:22), and the C-terminus may include substitutions and additions (“tail”), e.g., P/S/QA/T/ELQ/EY/I (SEQ ID NO:23), and optionally SG. For instance, the C-terminus can be either EISG (SEQ ID NO:24), EI, QY or Q. For the N-vectors, the N-terminus may be MAE, and in the C-vectors the N-terminal sequence or the mutant DhaA may be GSE or MAE. Tails include but are not limited to QY and EISG (SEQ ID NO:24).

### Example 2

### Sites Tolerant to Modification in Renilla Luciferase

Renilla luciferase constructs having inserted into sites tolerant to modification, e.g., between residues 91/92, 223/224 or 229/230, were prepared. They are: hRL(1-91)-4 amino acid peptide linker-RIIBetaB-4 amino acid peptide linker-hRL (92-311), hRL(1-91)-4 amino acid peptide linker-RIIBetaB-20 amino acid peptide linker-hRL992-311), hRL(1-91)-10 amino acid peptide linker-RIMetaB-4 amino acid linker-hRL(92-311), hRL(1-91)-42 amino acid peptide linker-hRL(92-311), hRL(1-223)-4 amino acid peptide linker-RIIBetaB-4 amino acid linker-hRL(224-311), hRL(1-223)-4 amino acid peptide linker-RIIBetaB-20 amino acid linker-hRL(224-311), hRL(1-223)-10 amino acid peptide linker-RIIBetaB-4 amino acid linker-hRL(224-311), hRL(1-223)-10 amino acid peptide linker-RIIBetaB-20 amino acid linker-hRL(224-311), hRL(1-223)-42 amino acid peptide linker-hRL(224-311), hRL(1-229)-4 amino acid peptide linker-RIIBetaB-4 amino acid linker-hRL(230-311), hRL(1-229)-4 amino acid peptide linker-RIIBetaB-20 amino acid linker-hRL(230-311), hRL(1-229)-42 amino acid peptide linker-hRL(230-311).

Protein was expressed from the constructs using the TnT T7 Coupled Wheat Germ Lysate System, 17 μL of TNT reaction was mixed with 17 μL of 300 mM HEPES/200 mM Thiourea (pH about 7.5) supplemented with 3.4 μL of 1 mM cAMP stock or dH₂O; reactions were allowed to incubate at room temperature for approximately 10 minutes. Ten μL of each sample was added to a 96 well plate well in triplicate and luminescence was measured using 100 μL of Renilla luciferase assay reagent on a Glomax luminometer. The hRL(1-91)-linker-RIIBetaB-linker-hRL(92-311) proteins were induced by 12-23 fold, the hRL(1-223)-linker RIIBetaB-linker-hRL(224-311) proteins were not induced and the hRL(1-229)-linker-RIffletaB-(230-311) proteins were induced about 2 to 9 fold. None of the 42 amino acid linker constructs were induced, nor were the full length Renilla luciferase construct or the “no DNA” controls.

Those sites and other sites potentially tolerant to modification are shown below.

site

31
42
69
111
151
169
193
208
251
259
274
91
223
229

For all but four of the constructs, the site was chosen because it was in a solvent exposed surface loop. Renilla luciferase may be employed as a model for sites tolerant to modification in other hydrolases such as dehalogenases, e.g., using 1BN6 (Rhodococcus sp.) and 2DHD (Xanthobacter autotrophicus) haloalkane dehalogenase crystal structures as templates. Solvent exposed surface loops may be more amenable to modification versus sites buried in the protein core or sites that are involved in alpha or beta structures. Thus, regions in a dehalogenase corresponding to those which are tolerant to modification in a Renilla luciferase, e.g., regions corresponding to residue 86 to 97, residue 96 to 116 or residue 218 to 235 of a Renilla luciferase, are useful to prepare “split” dehalogenase proteins for PCA or PCL.

### Example 3

The rapamycin-mediated FRB/FKBP protein-protein interaction and a mutant DhaA were employed in a PCL. FRB and FKBP will only interact when rapamycin is present. Therefore, if PCL is successful, the reconstituted reporter is labeled only when the fusion proteins are incubated together in the presence of rapamycin.

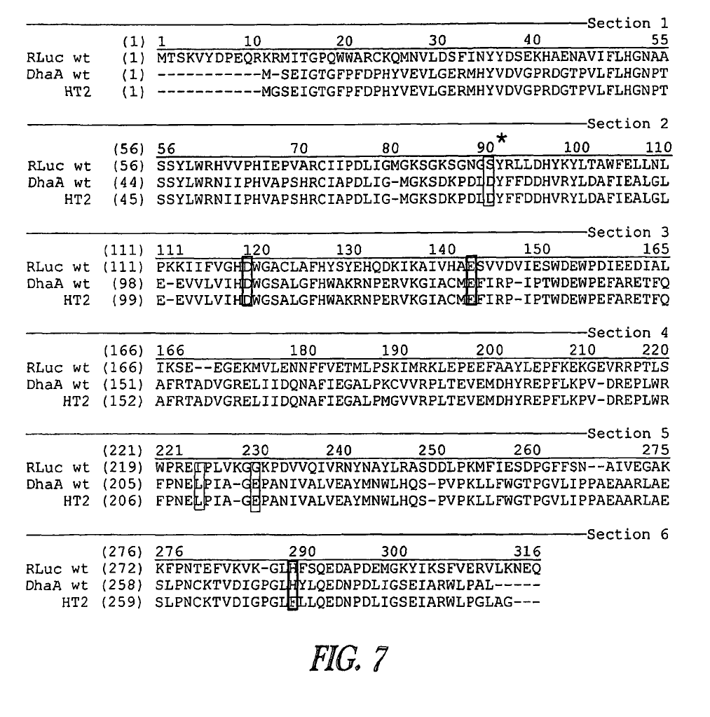

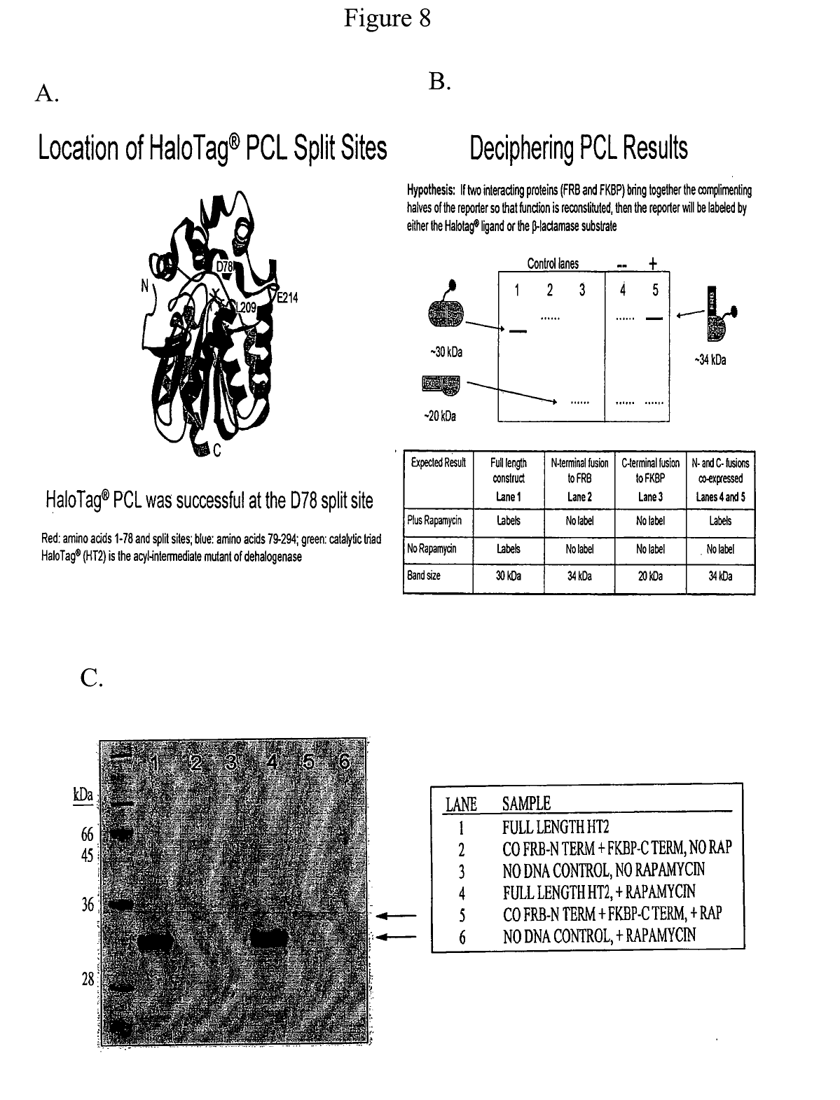

Two pF9 (Kan) vectors were generated which contained either FRB or FKBP ORF plus the linker sequence (GlyGlyGlyGlySer)₂ (SEQ ID NO:14 represents GlyGlyGlyGlySer) upstream of the SefI/PmeI sites. A mutant DhaA gene (HT2) at positions corresponding to those useful to prepare Renilla luciferase fragments for PCS (see Example 2 and FIG. 7) with FRB-N terminal and FKBP-C terminal fusions. HT2 N- and C-termini halves were amplified using PCR primers and cloned into the SefI/PmeI sites. PCL was performed in vitro by expressing each clone individually using RiboMax followed by Wheat Germ Plus reactions (HT2). Protein was expressed with or without FluoroTect™. FluoroTect™ labeling ensured that all proteins were expressed in approximately equal amounts (data not shown). Unlabeled proteins were then incubated alone or with the appropriated partner with or without 1 μM rapamycin. Ten μl of these products were then incubated with 0.1 μM of a TMR labeled ligand for the mutant dehalogenase, for 2 hours in the dark. All samples were then incubated at 70° C. for 5 minutes with 1×SDS/50 mM DTT loading buffer, followed by denaturing NuPAGE® gel electrophoresis. FIG. 8B shows expected results.

For transient transfections, CHO cells were plated in a 6 well plate and transfected in duplicate using TransIT®-CHO. The next day, cells were incubated +/−1 μM rapamycin for 2.5 hours followed by 1.0 μM HaloTag® TMR ligand for 1 hour. Cells were washed in PBS, trypsinized, pelleted and mechanically lysed in 200 ul PBS with protease inhibitor and RQDNase I. Normalized amounts of proteins were microwaved for 30 seconds on high and run on a denaturing NuPAGE® gel.

### Results

Co-incubation of FRB-N term (1-78)+FKBP-C term (79-294) retained TMR label only when incubated with rapamycin. Full length HT2 was also labeled, as expected. FluoroTect™ labeling indicated that all proteins were expressed equally (data not shown). Moreover, PCL mediated protein in CHO cells was labeled in the presence of rapamycin (FIG. 8C). There was also a small amount of rapamycin-independent PCL. Full length HT2 was labeled irrespective of rapamycin addition.

Thus, this technology has the potential to provide greater sensitivity for the detection of weak protein-protein interactions by accumulating label over time. Moreover, this technology can easily transition between in vitro, in vivo and in situ imaging studies using the same vector construct.

### Example 4

### Protein Complementation with Htv7 and Humanized Renilla Luciferase (hRL) in the FRB-N-Terminal Reporter Fragment+FKBP-C-Terminal Reporter Fragment Orientation

Many cellular signals are communicated and achieved through a network of cascading protein-protein interactions. Eventually, many of these signals result in a genetic response which can be monitored using gene reporter assays. The ability to assay cellular events closer to the primary event is desirable because it allows for a more “real-time” analysis of the cellular response and reduces the possibility of artifacts due to confounding factors at the later, downstream points.

To monitor protein-protein interactions, two fusion proteins are prepared. One fusion protein contains a portion of a reporter protein and a protein of interest (a first heterologous sequence, heterologous relative to the reporter protein, that interacts with another (second) heterologous sequence). The other fusion protein contains a portion of a protein that is functionally distinct from, but complements the portion of the reporter protein in the first fusion, and the second heterologous amino acid sequence. In one embodiment, one protein of interest is fused at the N- or C-terminus of a N-terminal or C-terminal portion of a Renilla luciferase, and the other protein of interest is fused at the N- or C-terminus of a C-terminal or N-terminal portion of a mutant dehalogenase, e.g., one referred to as HTv7. Interaction of the proteins of interest reconstitutes the activity of the Renilla luciferase and/or the HTv7 protein. Which activity is reconstituted depends on which portion of the protein the catalytic site (or in the case of HTv7, the former catalytic site) lies.

Renilla luciferase and HTv7 were chosen as models for the hybrid complementation system based on structural similarity. A structure based analysis of haloalkane dehalogenase (Rhodococcus sp.; Swiss Prot # P59336) and a homology model of Renilla luciferase using 1BN6 (Rhodococcus sp.) and 2DHD (Xanthobacter autotrophicus) haloalkane dehalogenase crystal structures as templates resulted in about 30% identity.

### Materials and Methods

The two proteins were split at two positions: residue 78/79 or 98/99 and 91/92 or 111/112, for HTv7 and Renilla luciferase, respectively. The Renilla luciferase “split” positions have been previously shown to be successful in a Renilla luciferase protein complementation assay (PCA) (Kaihara, et al., 2003, and Remy et al., 2005) (see also Example 2). In addition, successful protein complementation labeling (PCL) was demonstrated using HT2 (a mutant dehalogenase that is related to HTv7, see Example 1) at position 78/79 (Example 3). Moreover, successful induction by cAMP was demonstrated using circularly permuted Renilla luciferase-RIIBetaB biosensors where the Renilla luciferase gene was circularly permuted at positions corresponding to amino acid positions 91/92 and 111/112 (see U.S. application Ser. No. 11/732,105).

PCA was performed using the rapamycin dependent FRB/FKBP model system. Fusion proteins were made in the following orientation: FRB-N-terminal reporter half and FKBP-C-terminal reporter half. Site-directed mutagenesis (Stratagene QuickChange) was used to introduce the nucleotides “TA” into the pF3A vector (Promega), which created a NheI restriction site just upstream of the Se restriction site (termed “pF3A(TA)” in Table 1 below). The following two cassettes were then inserted between the NheI and SgfI restriction sites: [FRB—AscI restriction site—GGGGSGGGGS linker (SEQ ID NO:15 is linker)] and [FKBP—AscI restriction site—GGGGSGGGGS linker (SEQ ID NO:15 is linker)]. In between the SgfI and PmeI restriction sites of the FRB construct the following reporter fragments were inserted: HTv7 (amino acids 1-78), HTv7 (amino acids 1-98), hRL (amino acids 1-91) and hRL (amino acids 1-111). In between the SgfI and PmeI restriction sites of the FKBP construct the following reporter fragments were inserted: HTv7 (amino acids 79-297), HTv7 (amino acids 99-297), hRL (amino acids 92-311) and hRL (amino acids 112-311). In addition, the entire coding region of HTv7 (amino acids 1-297) and hRL (amino acids 1-311) were inserted in between the SgfI and PmeI restriction sites of the pF3A vector. Table 1 lists the constructs.

TABLE 1

| Construct | Vector | Type | Description | Designation |
|---|---|---|---|---|
| 201518.54.02 | pF3A | Full length | HTv7 (1-297) | FL HTv7 |
| 201518.45.A2 | pF3A(TA) | FRB - N term | FRB-HTv7 (1-78) | FRB-H78 |
| 201518.45.B9 | pF3A(TA) | FRB - N term | FRB-HTv7 (1-98) | FRB-H98 |
| 201518.45.C6 | pF3A(TA) | FKBP - C term | FKBP-HTv7 (79-297) | FKBP-H79 |
| 201518.45.E1 | pF3A(TA) | FKBP - C term | FKBP-HTv7 (99-297) | FKBP-H99 |
| 201518.45.01 | pF3A | Full length | hRL (1-311) | FL hRL |
| 201518.45.E9 | pF3A(TA) | FRB - N term | FRB-hRL (1-91) | FRB-R91 |
| 201518.73.D1 | pF3A(TA) | FRB - N term | FRB-hRL (1-111) | FRB-R111 |
| 201518.61.B1 | pF3A(TA) | FKBP - C term | FKBP-hRL (92-311) | FKBP-R92 |
| 201518.45.03 | pF3A(TA) | FKBP - C term | FKBP-hRL (112-311) | FKBP-R112 |

Proteins were co-expressed (or singly expressed for the full length HT and Renilla luciferase proteins and the FRB-N-terminal HTv7 or RL fragments or FKBP-C-terminal HTv7 or RL fragment only controls) using the TnT Sp6 High-Yield Protein Expression System (Promega). Two μg of total DNA was incubated at 25° C. for 2 hours with the master mix in 50 μl reactions as per the manufacturer's protocol with or without 2 μl of FluoroTect Green_{Lys} in vitro Translation labeling System (Promega) and with or without 1 μM rapamycin (BioMol). Five μl of the resultant non-FluoroTect labeled lysates were then incubated with 1 μM HT (DhaA) TMR ligand (Promega) for 2.5 hours at room temperature in the dark. Five μA of all lysates (with and without FluoroTect, with and without rapamycin) were then incubated with 5-10 U of RNase ONE Ribonuclease (Promega) for 15 minutes at room temperature. The lysates were then mixed with 1×LDS loading dye (Invitrogen), 60 μM DTT and water to 20 μl total volume. Samples were then size fractionated on a 4-12% Bis-Tris SDS PAGE gels (Invitrogen).

For the Renilla luciferase activity assay, ten μL lysate (with and without rapamycin) was diluted 1:1 in 2×HEPES/thiourea and 5 μL was placed in a 96-well plate well, in triplicate. Luminescence was measured by addition of 100 μL Renilla Luciferase Assay Reagent (Promega; R-LAR) by injectors.

### Results

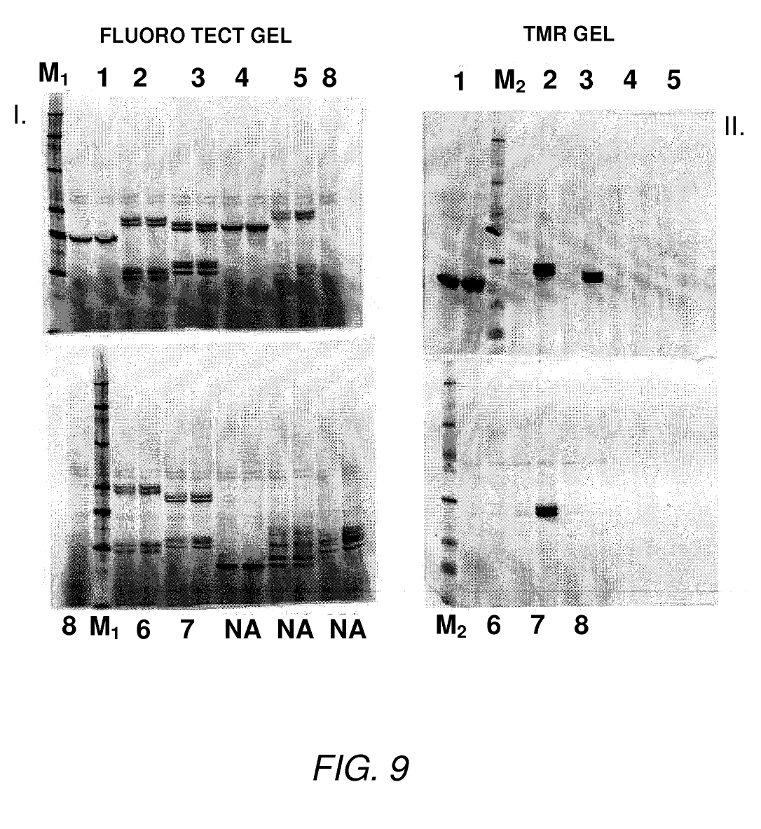

FIGS. 9A and 9B show that the N- and C-terminal reporter portions of HTv7 can reconstitute labeling activity in the presence of rapamycin at split sites H78/H79 and H98/H99. There is also some small amount of rapamycin independent labeling activity (FIG. 9A, lanes 2 and 3; FIG. 9B, lane 3). In addition, the N-terminal hRL fragment+the C-terminal HTv7 fragment can reconstitute labeling activity in the presence of rapamycin at split sites R91/H79 and R111/H99 (FIG. 9A, lane 7 and FIG. 9B, lane 7).

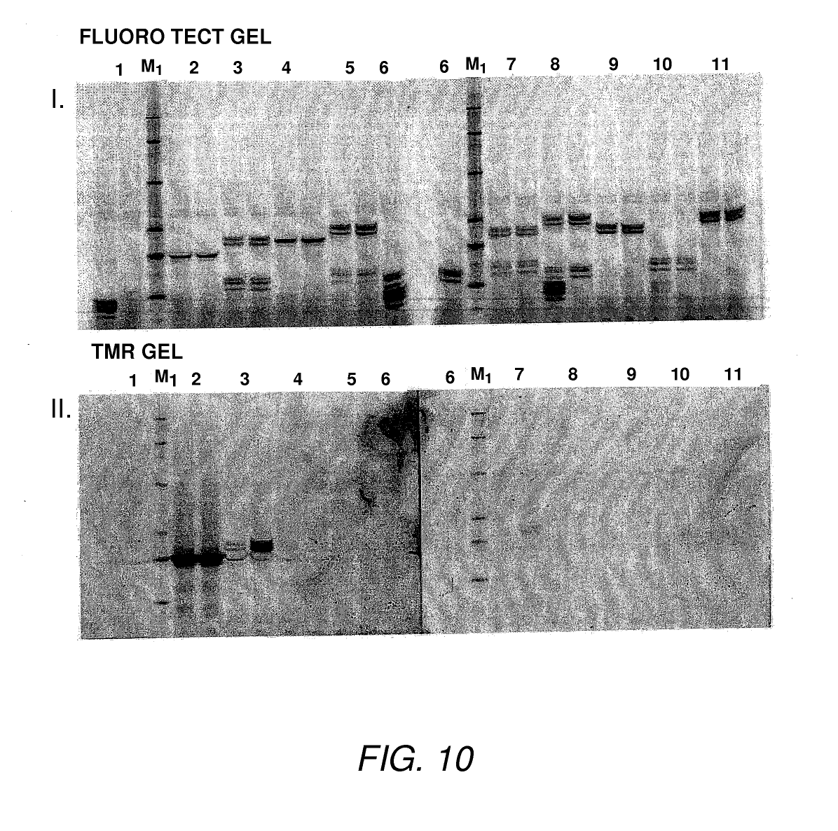

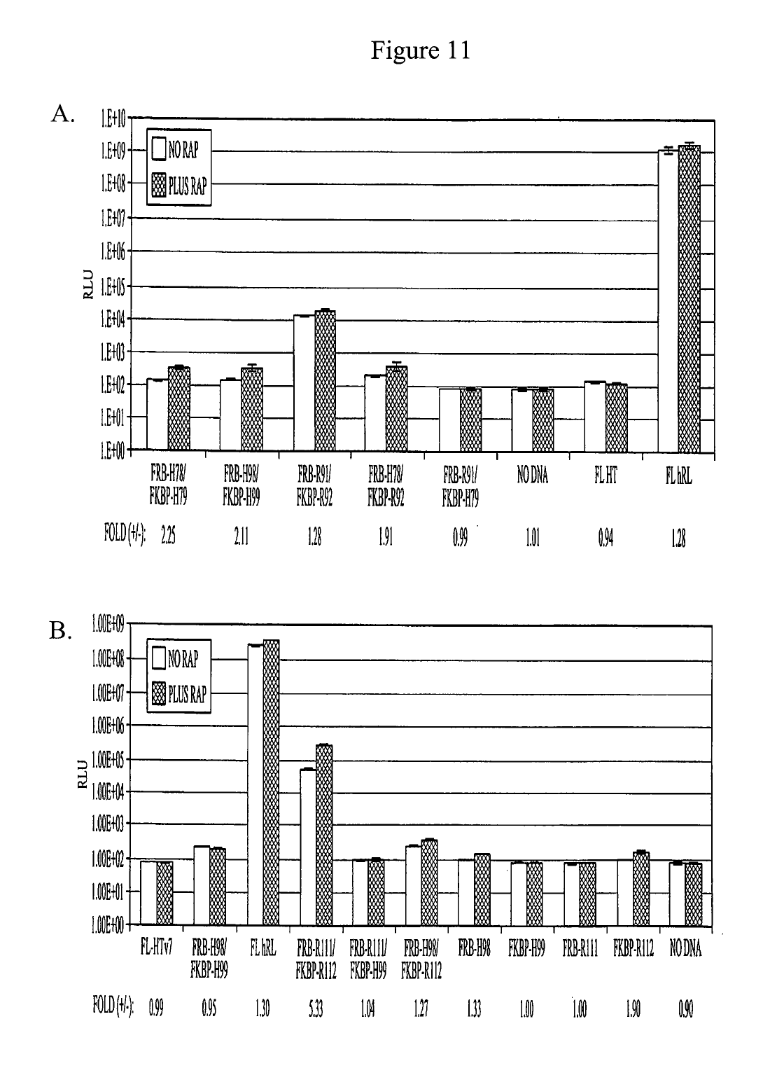

The results for the Renilla luciferase assay are shown in FIGS. 10A and 10B. None of the PCA constructs+rapamycin resulted in significant Renilla luciferase activity except for the FRB-R111+FKBP-R112 combination. This combination gave 5.3 fold more Renilla luciferase activity+rapamycin as compared to no rapamycin.

### Example 5

### Protein Complementation with HTv7 and Humanized Renilla Luciferase (hRL) in the N-Terminal Reporter Fragment—FRB+FKBP-C-Terminal Reporter Fragment Orientation

### Materials and Methods

PCA was performed using the rapamycin dependent FRB/FKBP model system. To test an “insertion-like” orientation, an additional set of fusion proteins were made in the pF3A vector (Promega) in the orientation: N-terminal reporter fragment—FRB. The following cassettes were then inserted in-between the SgfI and PmeI restriction sites: [C-terminal reporter fragment—GGSSGGGSGG (SEQ ID NO:21) linker (includes a Sad restriction site)—FRB]. The following N-terminal reporter fragments were inserted: HTv7 (amino acids 1-78), HTv7 (amino acids 1-98), hRL (amino acids 1-91) and hRL (amino acids 1-111). Table 2 lists the constructs.

TABLE 2

| Construct | Vector | Type | Description | Designation |
|---|---|---|---|---|
| 201518.172.H7 | pF3A | N term - FRB | HTv7 (1-78) -FRB | FRB-H78 |
| 201518.172.G10 | pF3A | N term - FRB | HTv7 (1-98) -FRB | FRB-H98 |
| 201518.176.01 | pF3A | N term - FRB | hRL (1-91)-FRB | FRB-R91 |
| 201518.158.A4 | pF3A | N term - FRB | hRL (1-111)-FRB | FRB-R111 |

Proteins were co-expressed (or singly expressed for the full length HaloTag and Renilla luciferase proteins) using the TnT Sp6 High-Yield Protein Expression System (Promega). Two μg of total DNA was incubated at 25° C. for 2 hours with the master mix in 50 μl reactions as per the manufacturer's protocol with or without 2 μl of FluoroTect Green_{Lys} in vitro Translation labeling System (Promega). Twenty μl of the resultant lysates (with and without FluoroTect) were then incubated with or without 1 μM rapamycin (BioMol) for 15 minutes at RT. Five μl of the non-FluoroTect labeled lysates were then incubated with 1 μM HT-TMR ligand (Promega) for about 45 minutes on ice in the dark. Five μl of the FluoroTect labeled lysates (with and without rapamycin) were then incubated with 5-10 U of RNase ONE Ribonuclease (Promega) for 15 minutes at RT. The lysates were then mixed with 1×LDS loading dye (Invitrogen) and water to 20 μl total volume. Samples were then size fractionated on a 4-20% Bis-HCl SDS PAGE gels (Bio-Rad).

For the Renilla activity assay, ten μL lysate (with and without rapamycin) was diluted 1:1 in 2×HEPES/thiourea and 5 μL was placed in a 96-well plate well, in triplicate. Luminescence was measured by addition of 100 μL Renilla Luciferase Assay Reagent (Promega; R-LAR) by injectors.

### Results

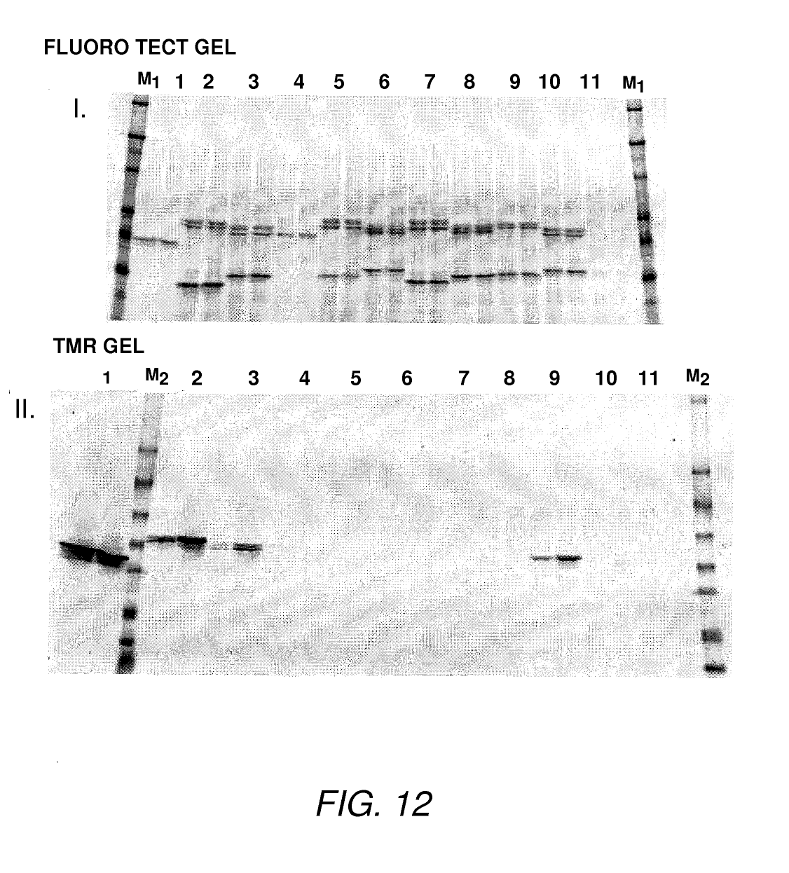

FIG. 12 shows that the N- and C-terminal fragments of HTv7 can reconstitute labeling activity in the presence of rapamycin at split sites H78/H79 and H98/H99 in the “insertion-like” orientation. There is also some small amount of rapamycin independent labeling activity (FIG. 12, lanes 2 and 3). In addition, the N-terminal hRL reporter fragment+the C-terminal HTv7 reporter fragment can reconstitute labeling activity in the presence of rapamycin at split sites R91/H79 and R111/H99 in the “insertion-like” orientation (FIG. 12, lanes 9 and 10). There is a small amount of rapamycin independent labeling with the R91/H79 combination (FIG. 12, lane 9).

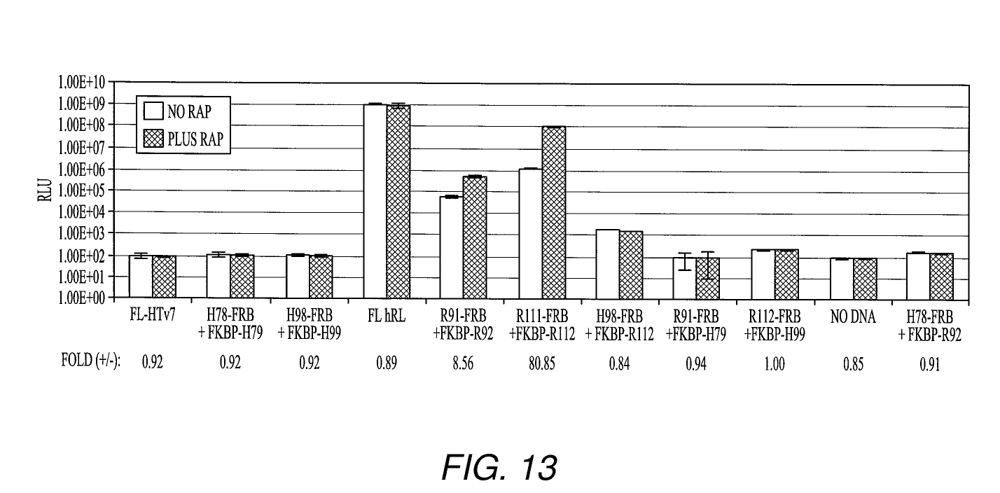

None of the PCA constructs+rapamycin resulted in significant Renilla luciferase activity except for the R91-FRB+FKBP-R92 and R111-FRB+FKBP-R112 combinations. These combinations gave 8.6 and 81 fold more Renilla luciferase activity+rapamycin as compared to no rapamycin, respectively (FIG. 13).

### Example 6

Protein Complementation with HTv7 and Humanized Renilla Luciferase (hRL) in the C-Terminal Fragment—FKBP+FRB-N-Terminal Fragment Orientation

### Materials and Methods

PCA was performed using the rapamycin dependent FRB/FKBP model system. To test a “CP-like” orientation, an additional set of fusion proteins were made in the pF3A vector (Promega) in the orientation: C-terminal reporter fragment—FKBP. The following cassettes were inserted in between the SgfI and PmeI restriction sites: [Met-C-terminal reporter fragment—GGSSGGGSGG (SEQ ID NO:21) linker (includes a Sad restriction site)—FKBP]. The following C-terminal reporter fragments were inserted: HTv7 (Met-amino acids 79-297), HTv7 (Met-amino acids 99-297), hRL (Met-amino acids 92-311) and hRL (Met-amino acids 112-311). Table 3 lists the constructs.

TABLE 3

| Construct | Vector | Type | Description | Designation |
|---|---|---|---|---|
| 201591.13.09 | pF3A | C term - FKBP | HTv7 (79-297)-FKBP | H79-FKBP |
| 201591.13.14 | pF3A | C term - FKBP | HTv7 (99-297)-FKBP | H99-FKBP |
| 201591.13.03 | pF3A | C term - FKBP | hRL (92-311)-FKBP | R92-FKBP |
| 201591.13.06 | pF3A | C term - FKBP | hRL (112-311)-FKBP | R112-FKBP |

Proteins were co-expressed (or singly expressed for the full length HaloTag and Renilla proteins) using the TnT Sp6 High-Yield Protein Expression System (Promega). Two μg of total DNA was incubated at 25° C. for 2 hours with the master mix in 50 μl reactions as per the manufacturer's protocol with or without 2 μl of FluoroTect Green_{Lys} in vitro Translation labeling System (Promega). Twenty μl of the resultant lysates (with and without FluoroTect) were then incubated with or without 1 μM rapamycin (BioMol) for 15 minutes at room temperature. Five μl of the non-FluoroTect labeled lysates were then incubated with 1 uM HaloTag TMR ligand (Promega) for about 45 minutes on ice in the dark. Five μl of the FluoroTect labeled lysates (with and without rapamycin) were then incubated with 5-10 U of RNase ONE Ribonuclease (Promega) for 15 minutes at RT. The lysates were then mixed with 1×LDS loading dye (Invitrogen) and water to 20 μl total volume. Samples were then size fractionated on a 4-20% Bis-HCl SDS PAGE gels (Bio-Rad).

For the Renilla luciferase activity assay, ten μL lysate (with and without rapamycin) was diluted 1:1 in 2×HEPES/thiourea and 5 μL was placed in a 96-well plate well, in triplicate. Luminescence was measured by addition of 100 μL Renilla Luciferase Assay Reagent (Promega; R-LAR) by injectors.

### Results

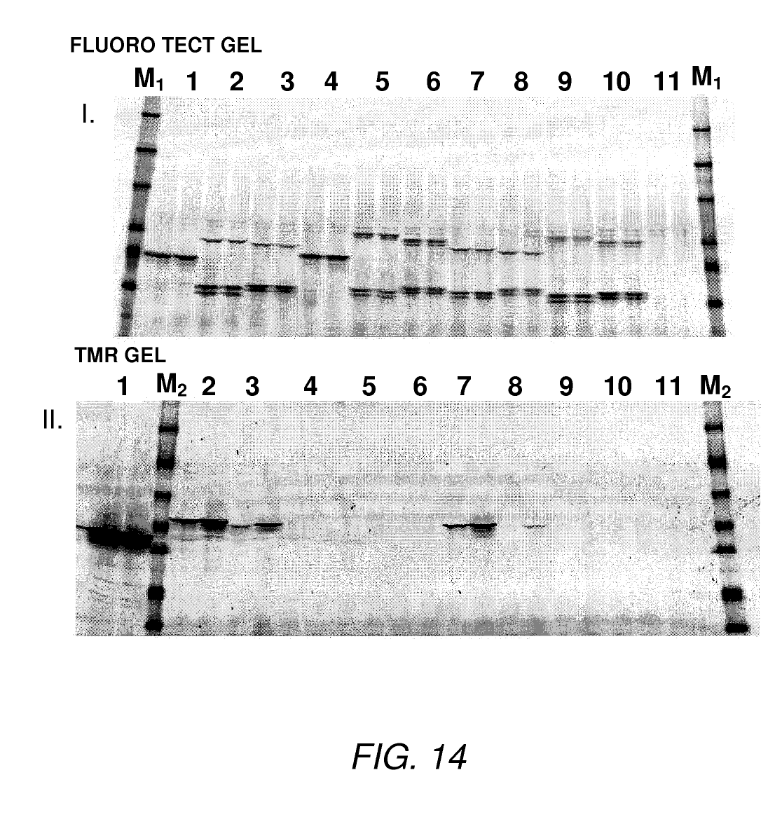

FIG. 14 shows that the N- and C-terminal reporter fragments of HTv7 can reconstitute labeling activity in the presence of rapamycin at split sites H79/H78 and H99/H98 in the “CP-like” orientation. There is also some small amount of rapamycin independent labeling activity (FIG. 14, lanes 2 and 3). In addition, the N-terminal hRL reporter fragment+the C-terminal HTv7 reporter fragment can reconstitute labeling activity in the presence of rapamycin at split sites H79/R91 and H99/R111 in the “CP-like” orientation (FIG. 14, lanes 7 and 8). There is a small amount of rapamycin independent labeling with the H79/R91 combination (FIG. 14, lane 7).

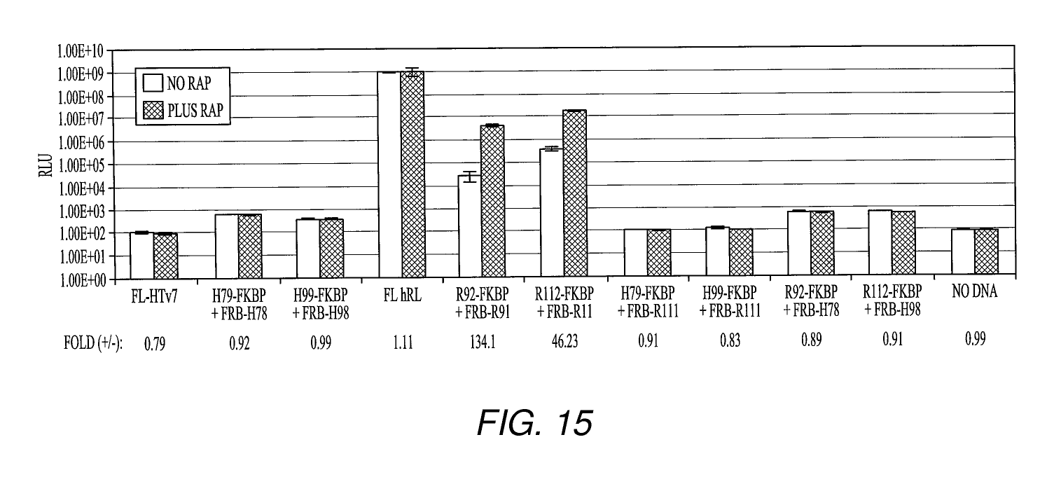

The results for Renilla luciferase activity are shown in FIG. 15. None of the PCA constructs+rapamycin resulted in significant Renilla activity except for the R92-FKBP+FRB-R91 and R111-FKBP+FRB-R112 combinations. These combinations gave 134- and 46-fold more Renilla luciferase activity+rapamycin as compared to no rapamycin, respectively (FIG. 15).

### REFERENCES

Cheltsov et al., J. Biol. Chem., 278:27945 (2003).
Chong et al., Gene, 192:271 (1997).
Einbond et al., FEBS Lett., 384:1 (1996).
Greene, Protecting Groups In Organic Synthesis; Wiley: New York, 1981
Hanks and Hunter, FASEB J, 9:576-595 (1995).
Harlow and Lane, In: Antibodies: A Laboratory Manual, Cold Spring Harbor Laboratory Press, p. 726 (1988)
Ilsley et al., Cell Signaling, 14:183 (2002).
Janssen et al., Eur. J. Biochem., 171:67 (1988).
Janssen et al., J. Bacteriol., 171:6791 (1989).
Jougard et al., Acta Crystallogr. D. Biol. Crystallogr., 58:2018 (2002).
Keuning et al., J. Bacteriol., 163:635 (1985).
Kwon et al., Anal. Chem., 76:5713 (2004).
Mayer and Baltimore, Trends Cell. Biol., 3:8 (1993).
Mils et al., Oncogene, 19:1257 (2000).
Murray et al., Nucleic Acids Res., 17:477 (1989).
Nagai et al., Proc. Natl. Acad. Sci. USA, 98:3197 (2001).
Nagata et al., Appl. Environ. Microbiol., 63:3707 (1997).
Ozawa et al, Analytical Chemistry, 73:2516 (2001).
Paulmurugan et al., Proc. Natl. Acad. Sci. USA, 99:3105 (2002).
Qureshi et al., J. Biol. Chem., 276:46422 (2001).
Sadowski, et al., Mol. Cell. Bio., 6:4396 (1986).
Sala-Newby et al., Biochem J., 279:727 (1991).
Sallis et al., J. Gen. Microbiol., 136:115 (1990).
Scholtz et al., J. Bacteriol., 169:5016 (1987).
Wada et al., Nucleic Acids Res., 18 Suppl:2367 (1990).
Waud et al, BBA, 1292:89 (1996).
Yokota et al., J. Bacteriol., 169:4049 (1987).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1-79. (canceled)

80. A composition comprising a first polynucleotide comprising an open reading frame for a first fusion protein having a first fragment of a dehalogenase and a first heterologous amino acid sequence, wherein the first fragment of the dehalogenase includes at least 20 contiguous amino acid residues of a full length dehalogenase which residues are capable of associating with a second fragment of a dehalogenase, wherein the complex formed by the association of the two fragments, but not the first dehalogenase fragment or the second dehalogenase fragment, is capable of stably binding a dehalogenase substrate for a corresponding full length, wild type dehalogenase, wherein the N- and/or C-termini of the first and second dehalogenase fragments are at a residue or in a region in a full length wild type dehalogenase sequence which is tolerant to modification, and wherein the first heterologous amino acid sequence is selected to directly or indirectly interacts with a molecule of interest.

81. The composition of claim 80 further comprising a second polynucleotide comprising an open reading frame for a second fusion protein comprising a second fragment of the dehalogenase and a second heterologous amino acid sequence, wherein the second dehalogenase fragment together with the first hydrolase fragment substantially corresponds in sequence to a mutant dehalogenase comprising at least one amino acid substitution at an amino acid residue in the corresponding full-length, wild-type dehalogenase that is associated with activating a water molecule which cleaves the bond formed between the corresponding full length, wild type dehalogenase and the dehalogenase substrate or at an amino acid residue in the corresponding full length, wild type dehalogenase that forms an ester intermediate with the dehalogenase substrate, and wherein the mutant dehalogenase forms a bond with a dehalogenase substrate which comprises one or more functional groups, which bond is more stable than the bond formed between the corresponding full length, wild type dehalogenase and the dehalogenase substrate which comprises the one or more functional groups.

82. The composition of claim 81 wherein the mutant dehalogenase comprises at least two amino acid substitutions relative to a corresponding full length wild type dehalogenase, wherein a second substitution is at an amino acid residue in the full length wild type dehalogenase that is within the active site cavity and within 3 to 5 Å of a dehalogenase substrate bound to the full length wild type dehalogenase.

83. The composition of claim 82 wherein the second substitution is to an amino acid which introduces one or more charges, introduces one or more hydrogen bonds, or reduces steric hindrance, thereby enhancing substrate binding.

84. The composition of claim 82 wherein one substitution is at a position corresponding to amino acid residue 106 or 272 of a Rhodococcus rhodochrous dehalogenase.

85. The composition of claim 82 wherein the second substitution is at a position corresponding to amino acid residue 175, 176 or 273 of a Rhodococcus rhodochrous dehalogenase.

86. The composition of claim 81 wherein the mutant dehalogenase has at least two substitutions at positions corresponding to positions 5, 11, 20, 30, 32, 58, 60, 65, 78, 80 87, 94, 109, 113, 117, 118, 124, 134, 136, 150, 151, 155, 157, 172, 187, 204, 221, 224, 227, 231, 250, 256, 263, 272, 277, 282, 291 or 292 in SEQ ID NO:1.

87. A plurality of expression vectors comprising,
a) a first expression vector comprising a first promoter operably linked to an open reading frame for a first fusion protein having a first fragment of a dehalogenase and a first heterologous amino acid sequence, wherein the first dehalogenase fragment includes at least 20 contiguous amino acid residues of a full length dehalogenase which residuas are capable of associating with a second fragment of a dehalogenase, wherein the complex formed by the association of the two dehalogenase fragments, but not the first hydrolase fragment or the second dehalogenase fragment, is capable of binding a dehalogenase substrate for a corresponding full length, wild type dehalogenase, wherein the N- and/or C-termini of the first dehalogenase fragment are at a residue or in a region in a full length, wild type dehalogenase sequence which is tolerant to modification, and wherein the first heterologous amino acid sequence is selected to directly or indirectly interacts with a molecule of interest; and
b) a second expression vector comprising a second promoter operably linked to an open reading frame for a second fusion protein comprising a nucleotide sequence encoding the second dehalogenase fragment and a second heterologous amino acid sequence which interacts with the first heterologous amino acid sequence, wherein the second dehalogenases fragment which together with the first dehalogenase fragment of the dehalogenase substantially corresponds in sequence to a mutant dehalogenase comprising at least one amino acid substitution at an amino acid residue in the corresponding full length, wild type dehalogenase that is associated with activating a water molecule which cleaves the bond formed between the corresponding full length, wild type dehalogenase and the dehalogenase substrate or at an amino acid residue in the corresponding full length, wild type dehalogenase that forms an ester intermediate with the substrate, wherein the mutant dehalogenase forms a bond with a dehalogenase substrate which comprises one or more functional groups, which bond is more stable than the bond formed between the corresponding full length, wild type dehalogenase and the dehalogenase substrate which comprises the one or more functional groups, and wherein the N- and/or C-termini of the second dehalogenase fragment are at a residue or in the region in a full length, wild type dehalogenase sequence which is tolerant to modification.

88. The plurality of vectors of claim 87 wherein the mutant dehalogenase comprises at least two amino acid substitutions relative to a corresponding full length, wild type dehalogenase, and wherein a second substitution is at an amino acid residue in the full length, wild type dehalogenase that is within the active site cavity and within 3 to 5 Å of a dehalogenase substrate bound to the full length, wild type dehalogenase.

89. The plurality of vectors of claim 88 wherein the second substitution is to an amino acid which introduces one or more charges, introduces one or more hydrogen bonds, or reduces steric hindrance, thereby enhancing substrate binding.

90. The plurality of vectors of claim 88 wherein one substitution is at a position corresponding to amino acid residue 106 or 272 of a Rhodococcus rhodochrous dehalogenase.

91. The plurality of vectors of claim 89 wherein the second substitution is at a position corresponding to amino acid residue 175, 176 or 273 of a Rhodococcus rhodochrous dehalogenase.

92. The plurality of vectors of claim 88 wherein the mutant dehalogenase has at least two substitutions at positions corresponding to positions 5, 11, 20, 30, 32, 58, 60, 65, 78, 80 87, 94, 109, 113, 117, 118, 124, 134, 136, 150, 151, 155, 157, 172, 187, 204, 221, 224, 227, 231, 250, 256, 263, 272, 277, 282, 291 or 292 in SEQ ID NO:1.

93. A method to detect an interaction between two proteins in a sample, comprising:
a) providing a sample comprising the composition of claim 81, and a dehalogenase substrate with at least one functional group under conditions effective to allow for association of the two proteins; and
b) detecting the presence, amount or location of the at least one functional group in the sample.

94. The method of claim 93 wherein the mutant dehalogenase comprises at least two amino acid substitutions relative to a corresponding full length, wild type dehalogenase, and wherein a second substitution is at an amino acid residue in the full length, wild type dehalogenase that is within the active site cavity and within 3 to 5 Å of a dehalogenase substrate bound to the full length, wild type hydrolase.

95. The method of claim 94 wherein the second substitution is to an amino acid which introduces one or more charges, introduces one or more hydrogen bonds, or reduces steric hindrance, thereby enhancing substrate binding.

96. The method of claim 94 wherein one substitution is at a position corresponding to amino acid residue 106 or 272 of a Rhodococcus rhodochrous dehalogenase.

97. The method of claim 95 wherein the second substitution is at a position corresponding to amino acid residue 175, 176 or 273 of a Rhodococcus rhodochrous dehalogenase.

98. The method of claim 93 wherein the mutant dehalogenase has at least two substitutions at positions corresponding to positions 5, 11, 20, 30, 32, 58, 60, 65, 78, 80 87, 94, 109, 113, 117, 118, 124, 134, 136, 150, 151, 155, 157, 172, 187, 204, 221, 224, 227, 231, 250, 256, 263, 272, 277, 282, 291 or 292 in SEQ ID NO:1.

99. The method of claim 93 wherein the sample further comprises one or more agents that alters the interaction of the two proteins.


SEQUENCE LISTING (51 sequences)

SEQ ID NO: 1 - protein, 293 aa, Rhodococcus rhodochrous
MSEIGTGFPFDPHYVEVLGERMHYVDVGPRDGTPVLFLHGNPTSSYLWRNIIPHVAPSHRCIAPDLIGMGKSDKPDLDYFFDDHVRYLDAFIEALGLEEVVLVIHDWGSALGFHWAKRNPERVKGIACMEFIRPIPTWDEWPEFARETFQAFRTADVGRELIIDQNAFIEGALPKCVVRPLTEVEMDHYREPFLKPVDREPLWRFPNELPIAGEPANIVALVEAYMNWLHQSPVPKLLFWGTPGVLIPPAEAARLAESLPNCKTVDIGPGLHYLQEDNPDLIGSEIARWLPAL

SEQ ID NO: 2 - protein, 6 aa, Artificial Sequence
  A synthetic peptide
LVPRES

SEQ ID NO: 3 - protein, 6 aa, Artificial Sequence
  A synthetic peptide
HHHHHH

SEQ ID NO: 4 - protein, 10 aa, Artificial Sequence
  A synthetic peptide
EQKLISEEDL

SEQ ID NO: 5 - protein, 8 aa, Artificial Sequence
  A synthetic peptide
DYKDDDDK

SEQ ID NO: 6 - protein, 8 aa, Artificial Sequence
  A synthetic peptide
WSHPQFEK

SEQ ID NO: 7 - protein, 9 aa, Artificial Sequence
  A synthetic peptide
YPYDVPDYA

SEQ ID NO: 8 - protein, 5 aa, Artificial Sequence
  A synthetic peptide
RYIRS

SEQ ID NO: 9 - protein, 4 aa, Artificial Sequence
  A synthetic peptide
FHHT

SEQ ID NO: 10 - protein, 17 aa, Artificial Sequence
  A synthetic peptide
WEAAAREACCRECCARA

SEQ ID NO: 11 - protein, 4 aa, Artificial Sequence
  A synthetic peptide
RRFS

SEQ ID NO: 12 - protein, 7 aa, Artificial Sequence
  A synthetic peptide
LRRASLG

SEQ ID NO: 13 - protein, 5 aa, Artificial Sequence
  A synthetic peptide
HHHHH

SEQ ID NO: 14 - protein, 5 aa, Artificial Sequence
  A synthetic peptide
GGGGS

SEQ ID NO: 15 - protein, 10 aa, Artificial Sequence
  A synthetic peptide
GGGGSGGGGS

SEQ ID NO: 16 - DNA, 951 bp, Artificial Sequence
  A synthetic oligonucleotide
nnnngctagccagctggcgcggatatcgccaccatgggatccgagattgggacagggttcccttttgatcctcactatgttgaagtgctgggggaaagaatgcactacgtggatgtggggcctagagatgggaccccagtgctgttcctccacgggaaccctacatctagctacctgtggagaaatattatacctcatgttgctcctagtcataggtgcattgctcctgatctgatcgggatggggaagtctgataagcctgacttagactacttttttgatgatcatgttcgatacttggatgctttcattgaggctctggggctggaggaggtggtgctggtgatacacgactgggggtctgctctggggtttcactgggctaaaaggaatccggagagagtgaaggggattgcttgcatggagtttattcgacctattcctacttgggatgaatggccagagtttgccagagagacatttcaagcctttagaactgccgatgtgggcagggagctgattatagaccagaatgctttcatcgagggggctctgcctaaatgtgtagtcagacctctcactgaagtagagatggaccattatagagagccctttctgaagcctgtggatcgcgagcctctgtggaggtttccaaatgagctgcctattgctggggagcctgctaatattgtggctctggtggaagcctatatgaactggctgcatcagagtccagtgcccaagctactcttttgggggactccgggagttctgattcctcctgccgaggctgctagactggctgaatccctgcccaattgtaagaccgtggacatcggccctgggctgttttacctccaagaggacaaccctgatctcatcgggtctgagatcgcacggtggctgcccgggctggccggctaatagttaattaagtaggcggccgcnnnn

SEQ ID NO: 17 - DNA, 876 bp, Artificial Sequence
  A synthetic oligonucleotide
tccgaaatcggtacaggcttccccttcgacccccattatgtggaagtcctgggcgagcgtatgcactacgtcgatgttggaccgcgggatggcacgcctgtgctgttcctgcacggtaacccgacctcgtcctacctgtggcgcaacatcatcccgcatgtagcaccgagtcatcggtgcattgctccagacctgatcgggatgggaaaatcggacaaaccagacctcgattatttcttcgacgaccacgtccgctacctcgatgccttcatcgaagccttgggtttggaagaggtcgtcctggtcatccacgactggggctcagctctcggattccactgggccaagcgcaatccggaacgggtcaaaggtattgcatgtatggaattcatccggcctatcccgacgtgggacgaatggccagaattcgcccgtgagaccttccaggccttccggaccgccgacgtcggccgagagttgatcatcgatcagaacgctttcatcgagggtgcgctcccgatgggggtcgtccgtccgcttacggaggtcgagatggaccactatcgcgagcccttcctcaagcctgttgaccgagagccactgtggcgattccccaacgagctgcccatcgccggtgagcccgcgaacatcgtcgcgctcgtcgaggcatacatgaactggctgcaccagtcacctgtcccgaagttgttgttctggggcacacccggcgtactgatccccccggccgaagccgcgagacttgccgaaagcctccccaactgcaagacagtggacatcggcccgggattgttcttgctccaggaagacaacccggaccttatcggcagtgagatcgcgcgctggctcccggcactc

SEQ ID NO: 18 - protein, 292 aa, Artificial Sequence
  A synthetic peptide
SEIGTGFPFDPHYVEVLGERMHYVDVGPRDGTPVLFLHGNPTSSYLWRNIIPHVAPSHRCIAPDLIGMGKSDKPDLDYFFDDHVRYLDAFIEALGLEEVVLVIHDWGSALGFHWAKRNPERVKGIACMEFIRPIPTWDEWPEFARETFQAFRTADVGRELIIDQNAFIEGALPMGVVRPLTEVEMDHYREPFLKPVDREPLWRFPNELPIAGEPANIVALVEAYMNWLHQSPVPKLLFWGTPGVLIPPAEAARLAESLPNCKTVDIGPGLFLLQEDNPDLIGSEIARWLPAL

SEQ ID NO: 19 - protein, 27 aa, Artificial Sequence
  A synthetic peptide
QYSGGGGSGGGGSGGGGENLYFQAIEL

SEQ ID NO: 20 - protein, 5 aa, Artificial Sequence
  A synthetic peptide
GPALA

SEQ ID NO: 21 - protein, 10 aa, Artificial Sequence
  A synthetic peptide
GGSSGGGSGG

SEQ ID NO: 22 - protein, 5 aa, Artificial Sequence
  A synthetic peptide
XXETG

SEQ ID NO: 23 - protein, 5 aa, Artificial Sequence
  A synthetic peptide
XXLXX

SEQ ID NO: 24 - protein, 4 aa, Artificial Sequence
  A synthetic peptide
EISG

SEQ ID NO: 25 - DNA, 885 bp, Artificial Sequence
  A synthetic oligonucleotide
atggcagaaatcggtactggctttccattcgacccccattatgtggaagtcctgggcgagcgcatgcactacgtcgatgttggtccgcgcgatggcacccctgtgctgttcctgcacggtaacccgacctcctcctacctgtggcgcaacatcatcccgcatgttgcaccgacccatcgctgcattgctccagacctgatcggtatgggcaaatccgacaaaccagacctgggttatttcttcgacgaccacgtccgctacctggatgccttcatcgaagccctgggtctggaagaggtcgtcctggtcattcacgactggggctccgctctgggtttccactgggccaagcgcaatccagagcgcgtcaaaggtattgcatgtatggagttcatccgccctatcccgacctgggacgaatggccagaatttgcccgcgagaccttccaggccttccgcaccaccgacgtcggccgcgagctgatcatcgatcagaacgcttttatcgagggtacgctgccgatgggtgtcgtccgcccgctgactgaagtcgagatggaccattaccgcgagccgttcctgaagcctgttgaccgcgagccactgtggcgcttcccaaacgagctgccaatcgccggtgagccagcgaacatcgtcgcgctggtcgaagaatacatgaactggctgcaccagtcccctgtcccgaagctgctgttctggggcaccccaggcgttctgatcccaccggccgaagccgctcgcctggccgaaagcctgcctaactgcaagactgtggacatcggcccgggtctgaattttctgcaagaagacaacccggacctgatcggcagcgagatcgcgcgctggctgtcgacgctgcaatat

SEQ ID NO: 26 - protein, 295 aa, Artificial Sequence
  A synthetic peptide
MAEIGTGFPFDPHYVEVLGERMHYVDVGPRDGTPVLFLHGNPTSSYLWRNIIPHVAPTHRCIAPDLIGMGKSDKPDLGYFFDDHVRYLDAFIEALGLEEVVLVIHDWGSALGFHWAKRNPERVKGIACMEFIRPIPTWDEWPEFARETFQAFRTTDVGRELIIDQNAFIEGTLPMGVVRPLTEVEMDHYREPFLKPVDREPLWRFPNELPIAGEPANIVALVEEYMNWLHQSPVPKLLFWGTPGVLIPPAEAARLAESLPNCKTVDIGPGLNFLQEDNPDLIGSEIARWLSTLQY

SEQ ID NO: 27 - DNA, 885 bp, Artificial Sequence
  A synthetic oligonucleotide
atggcagaaatcggtactggctttccattcgacccccattatgtggaagtcctgggcgagcgcatgcactacgtcgatgttggtccgcgcgatggcacccctgtgctgttcctgcacggtaacccgacctcctcctacctgtggcgcaacatcatcccgcatgttgcaccgacccatcgctgcattgctccagacctgatcggtatgggcaaatccgacaaaccagacctgggttatttcttcgacgaccacgtccgctacctggatgccttcatcgaagccctgggtctggaagaggtcgtcctggtcattcacgactggggctccgctctgggtttccactgggccaagcgcaatccagagcgcgtcaaaggtattgcatgtatggagttcatccgccctatcccgacctgggacgaatggccagaatttgcccgcgagaccttccaggccttccgcaccaccgacgtcggccgcgagctgatcatcgatcagaacgcttttatcgagggtacgctgccgatgggtgtcgtccgcccgctgactgaagtcgagatggaccattaccgcgagccgttcctgaagcctgttgaccgcgagccactgtggcgcttcccaaacgagctgccaatcgccggtgagccagcgaacatcgtcgcgctggtcgaagaatacatgaactggctgcaccagtcccctgtcccgaagctgctgttctggggcaccccaggcgttctgatcccaccggccgaagccgctcgcctggccgaaagcctgcctaactgcaagactgtggacatcggcccgggtctgaatctgctgcaagaagacaacccggacctgatcggcagcgagatcgcgcgctggctgtcgacgctgcaatat

SEQ ID NO: 28 - protein, 295 aa, Artificial Sequence
  A synthetic peptide
MAEIGTGFPFDPHYVEVLGERMHYVDVGPRDGTPVLFLHGNPTSSYLWRNIIPHVAPTHRCIAPDLIGMGKSDKPDLGYFFDDHVRYLDAFIEALGLEEVVLVIHDWGSALGFHWAKRNPERVKGIACMEFIRPIPTWDEWPEFARETFQAFRTTDVGRELIIDQNAFIEGTLPMGVVRPLTEVEMDHYREPFLKPVDREPLWRFPNELPIAGEPANIVALVEEYMNWLHQSPVPKLLFWGTPGVLIPPAEAARLAESLPNCKTVDIGPGLNLLQEDNPDLIGSEIARWLSTLQY

SEQ ID NO: 29 - DNA, 885 bp, Artificial Sequence
  A synthetic oligonucleotide
atggcagaaatcggtactggctttccattcgacccccattatgtggaagtcctgggcgagcgcatgcactacgtcgatgttggtccgcgcgatggcacccctgtgctgttcctgcacggtaacccgacctcctcctacctgtggcgcaacatcatcccgcatgttgcaccgacccatcgctgcattgctccagacctgatcggtatgggcaaatccgacaaaccagacctgggttatttcttcgacgaccacgtccgcttcctggatgccttcatcgaagccctgggtctggaagaggtcgtcctggtcattcacgactggggctccgctctgggtttccactgggccaagcgcaatccagagcgcgtcaaaggtattgcatgtatggagttcatccgccctatcccgacctgggacgaatggccagaatttgcccgcgagaccttccaggccttccgcaccaccgacgtcggccgcgagctgatcatcgatcagaacgcttttatcgagggtacgctgccgatgggtgtcgtccgcccgctgactgaagtcgagatggaccattaccgcgagccgttcctgaagcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccgaaagcctgcctaactgcaagactgtggacatcggcccgggtctgaattttctgcaagaagacaacccggacctgatcggcagcgagatcgcgcgctggctgcaggagctgcaatat

SEQ ID NO: 30 - protein, 295 aa, Artificial Sequence
  A synthetic peptide
MAEIGTGFPFDPHYVEVLGERMHYVDVGPRDGTPVLFLHGNPTSSYLWRNIIPHVAPTHRCIAPDLIGMGKSDKPDLGYFFDDHVRFLDAFIEALGLEEVVLVIHDWGSALGFHWAKRNPERVKGIACMEFIRPIPTWDEWPEFARETFQAFRTTDVGRELIIDQNAFIEGTLPMGVVRPLTEVEMDHYREPFLKPVDREPLWRFPNELPIAGEPANIVALVEEYMDWLHQSPVPKLLFWGTPGVLIPPAEAARLAESLPNCKTVDIGPGLNFLQEDNPDLIGSEIARWLQELQY

SEQ ID NO: 31 - DNA, 885 bp, Artificial Sequence
  A synthetic oligonucleotide
atggcagaaatcggtactggctttccattcgacccccattatgtggaagtcctgggcgagcgcatgcactacgtcgatgttggtccgcgcgatagcacccctgtgctgttcctgcacggtaacccgacctcctcctacctgtggcgcaacatcatcccgcatgttgcaccgacccatcgctgcattgctccagacctgatcggtatgggcaaatccgacaaaccagacctgggttatttcttcgacgaccacgtccgcttcctggatgccttcatcgaagccctgggtctggaagaggtcgtcctggtcattcacgactggggctccgctctgggtttccactgggccaagcgcaatccagagcgcgtcaaaggtattgcatgtatggagttcatccgccctatcccgacctgggacgaatggccagaatttgcccgcgagaccttccaggccttccgcaccaccgacgtcggccgcgagctgatcatcgatcagaacgcttttatcgagggtacgctgccgatgggtgtcgtccgcccgctgactgaagtcgagatggaccattaccgcgagccgttcctgaagcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccgaaagcctgcctaactgcaagactgtggacatcggcccgggtctgaatctgctgcaagaagacaacccggacctgatcggcagcgagatcgcgcgctggctgcaggagctgcaatat

SEQ ID NO: 32 - protein, 295 aa, Artificial Sequence
  A synthetic peptide
MAEIGTGFPFDPHYVEVLGERMHYVDVGPRDSTPVLFLHGNPTSSYLWRNIIPHVAPTHRCIAPDLIGMGKSDKPDLGYFFDDHVRFLDAFIEALGLEEVVLVIHDWGSALGFHWAKRNPERVKGIACMEFIRPIPTWDEWPEFARETFQAFRTTDVGRELIIDQNAFIEGTLPMGVVRPLTEVEMDHYREPFLKPVDREPLWRFPNELPIAGEPANIVALVEEYMDWLHQSPVPKLLFWGTPGVLIPPAEAARLAESLPNCKTVDIGPGLNLLQEDNPDLIGSEIARWLQELQY

SEQ ID NO: 33 - DNA, 885 bp, Artificial Sequence
  A synthetic oligonucleotide
atggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctgcaatat

SEQ ID NO: 34 - protein, 295 aa, Artificial Sequence
  A synthetic peptide
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLQY

SEQ ID NO: 35 - DNA, 891 bp, Artificial Sequence
  A synthetic oligonucleotide
atggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctggagatttccgga

SEQ ID NO: 36 - protein, 297 aa, Artificial Sequence
  A synthetic peptide
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 37 - DNA, 882 bp, Artificial Sequence
  A synthetic oligonucleotide
tccgaaatcggtactggctttccattcgacccccattatgtggaagtcctgggcgagcgcatgcactacgtcgatgttggtccgcgcgatggcacccctgtgctgttcctgcacggtaacccgacctcctcctacctgtggcgcaacatcatcccgcatgttgcaccgacccatcgctgcattgctccagacctgatcggtatgggcaaatccgacaaaccagacctgggttatttcttcgacgaccacgtccgctacctggatgccttcatcgaagccctgggtctggaagaggtcgtcctggtcattcacgactggggctccgctctgggtttccactgggccaagcgcaatccagagcgcgtcaaaggtattgcatgtatggagttcatccgccctatcccgacctgggacgaatggccagaatttgcccgcgagaccttccaggccttccgcaccaccgacgtcggccgcgagctgatcatcgatcagaacgcttttatcgagggtacgctgccgatgggtgtcgtccgcccgctgactgaagtcgagatggaccattaccgcgagccgttcctgaagcctgttgaccgcgagccactgtggcgcttcccaaacgagctgccaatcgccggtgagccagcgaacatcgtcgcgctggtcgaagaatacatgaactggctgcaccagtcccctgtcccgaagctgctgttctggggcaccccaggcgttctgatcccaccggccgaagccgctcgcctggccgaaagcctgcctaactgcaagactgtggacatcggcccgggtctgaattttctgcaagaagacaacccggacctgatcggcagcgagatcgcgcgctggctgtcgacgctgcaatat

SEQ ID NO: 38 - protein, 294 aa, Artificial Sequence
  A synthetic peptide
SEIGTGFPFDPHYVEVLGERMHYVDVGPRDGTPVLFLHGNPTSSYLWRNIIPHVAPTHRCIAPDLIGMGKSDKPDLGYFFDDHVRYLDAFIEALGLEEVVLVIHDWGSALGFHWAKRNPERVKGIACMEFIRPIPTWDEWPEFARETFQAFRTTDVGRELIIDQNAFIEGTLPMGVVRPLTEVEMDHYREPFLKPVDREPLWRFPNELPIAGEPANIVALVEEYMNWLHQSPVPKLLFWGTPGVLIPPAEAARLAESLPNCKTVDIGPGLNFLQEDNPDLIGSEIARWLSTLQY

SEQ ID NO: 39 - DNA, 882 bp, Artificial Sequence
  A synthetic oligonucleotide
tccgaaatcggtactggctttccattcgacccccattatgtggaagtcctgggcgagcgcatgcactacgtcgatgttggtccgcgcgatggcacccctgtgctgttcctgcacggtaacccgacctcctcctacctgtggcgcaacatcatcccgcatgttgcaccgacccatcgctgcattgctccagacctgatcggtatgggcaaatccgacaaaccagacctgggttatttcttcgacgaccacgtccgctacctggatgccttcatcgaagccctgggtctggaagaggtcgtcctggtcattcacgactggggctccgctctgggtttccactgggccaagcgcaatccagagcgcgtcaaaggtattgcatgtatggagttcatccgccctatcccgacctgggacgaatggccagaatttgcccgcgagaccttccaggccttccgcaccaccgacgtcggccgcgagctgatcatcgatcagaacgcttttatcgagggtacgctgccgatgggtgtcgtccgcccgctgactgaagtcgagatggaccattaccgcgagccgttcctgaagcctgttgaccgcgagccactgtggcgcttcccaaacgagctgccaatcgccggtgagccagcgaacatcgtcgcgctggtcgaagaatacatgaactggctgcaccagtcccctgtcccgaagctgctgttctggggcaccccaggcgttctgatcccaccggccgaagccgctcgcctggccgaaagcctgcctaactgcaagactgtggacatcggcccgggtctgaatctgctgcaagaagacaacccggacctgatcggcagcgagatcgcgcgctggctgtcgacgctgcaatat

SEQ ID NO: 40 - protein, 294 aa, Artificial Sequence
  A synthetic peptide
SEIGTGFPFDPHYVEVLGERMHYVDVGPRDGTPVLFLHGNPTSSYLWRNIIPHVAPTHRCIAPDLIGMGKSDKPDLGYFFDDHVRYLDAFIEALGLEEVVLVIHDWGSALGFHWAKRNPERVKGIACMEFIRPIPTWDEWPEFARETFQAFRTTDVGRELIIDQNAFIEGTLPMGVVRPLTEVEMDHYREPFLKPVDREPLWRFPNELPIAGEPANIVALVEEYMNWLHQSPVPKLLFWGTPGVLIPPAEAARLAESLPNCKTVDIGPGLNLLQEDNPDLIGSEIARWLSTLQY

SEQ ID NO: 41 - DNA, 882 bp, Artificial Sequence
  A synthetic oligonucleotide
tccgaaatcggtactggctttccattcgacccccattatgtggaagtcctgggcgagcgcatgcactacgtcgatgttggtccgcgcgatggcacccctgtgctgttcctgcacggtaacccgacctcctcctacctgtggcgcaacatcatcccgcatgttgcaccgacccatcgctgcattgctccagacctgatcggtatgggcaaatccgacaaaccagacctgggttatttcttcgacgaccacgtccgcttcctggatgccttcatcgaagccctgggtctggaagaggtcgtcctggtcattcacgactggggctccgctctgggtttccactgggccaagcgcaatccagagcgcgtcaaaggtattgcatgtatggagttcatccgccctatcccgacctgggacgaatggccagaatttgcccgcgagaccttccaggccttccgcaccaccgacgtcggccgcgagctgatcatcgatcagaacgcttttatcgagggtacgctgccgatgggtgtcgtccgcccgctgactgaagtcgagatggaccattaccgcgagccgttcctgaagcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccgaaagcctgcctaactgcaagactgtggacatcggcccgggtctgaattttctgcaagaagacaacccggacctgatcggcagcgagatcgcgcgctggctgcaggagctgcaatat

SEQ ID NO: 42 - protein, 294 aa, Artificial Sequence
  A synthetic peptide
SEIGTGFPFDPHYVEVLGERMHYVDVGPRDGTPVLFLHGNPTSSYLWRNIIPHVAPTHRCIAPDLIGMGKSDKPDLGYFFDDHVRFLDAFIEALGLEEVVLVIHDWGSALGFHWAKRNPERVKGIACMEFIRPIPTWDEWPEFARETFQAFRTTDVGRELIIDQNAFIEGTLPMGVVRPLTEVEMDHYREPFLKPVDREPLWRFPNELPIAGEPANIVALVEEYMDWLHQSPVPKLLFWGTPGVLIPPAEAARLAESLPNCKTVDIGPGLNFLQEDNPDLIGSEIARWLQELQY

SEQ ID NO: 43 - DNA, 882 bp, Artificial Sequence
  A synthetic oligonucleotide
tccgaaatcggtactggctttccattcgacccccattatgtggaagtcctgggcgagcgcatgcactacgtcgatgttggtccgcgcgatagcacccctgtgctgttcctgcacggtaacccgacctcctcctacctgtggcgcaacatcatcccgcatgttgcaccgacccatcgctgcattgctccagacctgatcggtatgggcaaatccgacaaaccagacctgggttatttcttcgacgaccacgtccgcttcctggatgccttcatcgaagccctgggtctggaagaggtcgtcctggtcattcacgactggggctccgctctgggtttccactgggccaagcgcaatccagagcgcgtcaaaggtattgcatgtatggagttcatccgccctatcccgacctgggacgaatggccagaatttgcccgcgagaccttccaggccttccgcaccaccgacgtcggccgcgagctgatcatcgatcagaacgcttttatcgagggtacgctgccgatgggtgtcgtccgcccgctgactgaagtcgagatggaccattaccgcgagccgttcctgaagcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccgaaagcctgcctaactgcaagactgtggacatcggcccgggtctgaatctgctgcaagaagacaacccggacctgatcggcagcgagatcgcgcgctggctgcaggagctgcaatat

SEQ ID NO: 44 - protein, 294 aa, Artificial Sequence
  A synthetic peptide
SEIGTGFPFDPHYVEVLGERMHYVDVGPRDSTPVLFLHGNPTSSYLWRNIIPHVAPTHRCIAPDLIGMGKSDKPDLGYFFDDHVRFLDAFIEALGLEEVVLVIHDWGSALGFHWAKRNPERVKGIACMEFIRPIPTWDEWPEFARETFQAFRTTDVGRELIIDQNAFIEGTLPMGVVRPLTEVEMDHYREPFLKPVDREPLWRFPNELPIAGEPANIVALVEEYMDWLHQSPVPKLLFWGTPGVLIPPAEAARLAESLPNCKTVDIGPGLNLLQEDNPDLIGSEIARWLQELQY

SEQ ID NO: 45 - DNA, 882 bp, Artificial Sequence
  A synthetic oligonucleotide
tccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctgcaatat

SEQ ID NO: 46 - protein, 294 aa, Artificial Sequence
  A synthetic peptide
SEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLQY

SEQ ID NO: 47 - DNA, 888 bp, Artificial Sequence
  A synthetic oligonucleotide
tccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctggagatttccgga

SEQ ID NO: 48 - protein, 296 aa, Artificial Sequence
  A synthetic peptide
SEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 49 - protein, 311 aa, Renilla reniformis
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 50 - protein, 293 aa, Mycobacterium GP1
MSEIGTGFPFDPHYVEVLGERMHYVDVGPRDGTPVLFLHGNPTSSYLWRNIIPHVAPSHRCIAPDLIGMGKSDKPDLDYFFDDHVRYLDAFIEALGLEEVVLVIHDWGSALGFHWAKRNPERVKGIACMEFIRPIPTWDEWPEFARETFQAFRTADVGRELIIDQNAFIEGALPKFVVRPLTEVEMDHYREPFLKPVDREPLWRFPNELPIAGEPANIVALVEAYMNWLHQSPVPKLLFWGTPGVLISPAEAARLAESLPNCKTVDIGPGLHFLQEDNPDLIGSEIARWLPAL

SEQ ID NO: 51 - protein, 296 aa, Artificial Sequence
  A synthetic peptide
MGSEIGTGFPFDPHYVEVLGERMHYVDVGPRDGTPVLFLHGNPTSSYLWRNIIPHVAPSHRCIAPDLIGMGKSDKPDLDYFFDDHVRYLDAFIEALGLEEVVLVIHDWGSALGFHWAKRNPERVKGIACMEFIRPIPTWDEWPEFARETFQAFRTADVGRELIIDQNAFIEGALPMGVVRPLTEVEMDHYREPFLKPVDREPLWRFPNELPIAGEPANIVALVEAYMNWLHQSPVPKLLFWGTPGVLIPPAEAARLAESLPNCKTVDIGPGLFLLQEDNPDLIGSEIARWLPGLAG